(19) United States
(12) Patent Application Publication
(10) Pub. No.: US 2007/0043104 A1
(43) Pub. Date: Feb. 22, 2007

(54) UII-modulating compounds and their use

(76) Inventors: Ingrid Kristina Luthman, Goteborg (SE); Fredrik Lehmann, Sigtuna (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/449,411

(22) Filed: Jun. 8, 2006

Related U.S. Application Data
(60) Provisional application No. 60/690,312, filed on Jun. 10, 2005.

(51) Int. Cl. A61K 31/405; C07D 209/18; A61K 31/24; C07C 229/20

(52) U.S. Cl. 514/419; 514/534; 548/495; 560/37

(57) ABSTRACT

Disclosed herein are novel aromatic-containing compounds and methods for using various aromatic-containing compounds for treatment and prevention of diseases and disorders related to the Urotensin II receptor.

### BACKGROUND OF THE INVENTION

1. Field of the Invention

This invention relates to the fields of organic chemistry, pharmaceutical chemistry, biochemistry, molecular biology and medicine. In particular it relates to compounds that modulate the activity of the human Urotensin II receptor (UII), and to the use of the compounds for the treatment and prevention of diseases and disorders related to UII. Compounds that modulate the activity of the human UII receptor have also been described in U.S. Provisional Patent Application No. 60/690,312, entitled “UII-MODULATING COMPOUNDS AND THEIR USE,” filed Jun. 10, 2005, the disclosure of which is hereby incorporated by reference in its entirety.

2. Description of the Related Art

Urotensin II is an endogenous peptide agonist for a recently identified human G-protein coupled receptor. The human receptor is homologous to the rat orphan receptor GPR14.

Urotensin II is a cyclic neuropeptide found to be a potent vasoconstrictor in some systems and a vasodilator in others. The peptide is expressed in the motor neurons of the CNS, smooth muscle cells of the bladder and muscle cells of the heart. Its sequence is highly conserved among species, consisting of 11 amino acids in humans, 12 amino acids in fish, and 13 in frogs, with a fully conserved cyclic region from fish to humans.

The natural endogenous ligand, urotensin II, has been found to modulate the function of the urotensin II receptor. There is therefore a need in the art for non-endogenous ligands and modulators of the urotensin II receptor at least for use as medicaments.

Several responses to urotensin II have been observed in tissues and animals that may indicate physiological functions for this signaling molecule and its receptor and may indicate therapeutic uses of modulators of this system.

Human urotensin II has been reported as a potent spasmogen of primate airway smooth muscle and its contractile profile with pulmonary vascular tissue has showed that there were regional differences in its efficacy, with potent contractile activity on pulmonary arteries while having no effect in, tissues distal from the atria (Br. J. Pharmacol., 131(1); 10-12).

Human urotensin II has been reported as an endothelium-dependent vasodilator in rat small arteries (Br. J. Pharmacol., 130(8); 1865-1870). The human urotensin II peptide acts as a vasoconstrictor of rat and primate aorta, and induced a large increase in peripheral resistance in the circulation of primates along with a dramatic decrease in heart rate (Nature, 401; 282-286). In anesthetized rats, urotensin II peptide induced a decrease in blood pressure (General and Comparative Endocrinology 64; 435-439, Neuroendocrinol. Lett. 14(5); 357-363). These results suggest that modulators of urotensin II and its receptor may alter cardiovascular function, particularly heart rate, cardiac output, peripheral resistance and arterial pressure. (Russell, F. D. Emerging roles of urotensin-II in cardiovascular disease. Pharmacol. Therapeut. (2004) 103, 223-243; Kruger, S.; Graf, J.; Kunz, D.; Stickel, T.; Merx, M. W.; Hanrath, P.; Janssens, U. Urotensin II in patients with chronic heart failure. Eur. J. Heart Fail. (2005) 7, 475-478; Bousette, N.; Patel, L.; Douglas, S. A.; Ohlstein, E. H.; Giaid, A. Increased expression of urotensin II and its cognate receptor GPR14 in atherosclerotic lesions of the human aorta. Atherosclerosis (2004) 176, 117-123).

Indications are that the physiological role of urotensin II in mammals is strongly tissue dependent. The mRNA for the human urotensin II receptor is widely expressed in human tissue and is most abundant in heart and pancreas. The cardiovascular tissue of the left atrium and ventricle of the heart, and arterial tissue such as in the aorta are especially rich in expression of the urotensin II receptor. Moreover, the receptor is also distributed within the smooth muscle cells of the bladder, coronary arteries, and the aorta, the endothelial cells of the coronary artery and umbilical vein, and the motor neurons of the spinal cord. The distribution of the pro-pre-urotensin II mRNA in the human central nervous system is restricted primarily to the medulla oblongata of the brain and the spinal cord with the urotensin II-like immunoreactivity localized to motor neurons of the ventral horn. The distribution of the pro-pre-urotensin II mRNA in peripheral tissue is primarily restricted to the adrenal glands, the kidneys and the spleen. Accordingly, the UII receptor has a potential role in diseases such as renal failure, and diabetes. (Douglas, S.; Dhanak, D.; Johns, D. G. From ‘gills to pills’: urotensin II as a regulator of mammalian cardiorenal function. Trends in Pharmacological sciences (2004) 25, 76-85; Wenyi, Z.; Suzuki, S.; Hirai, M.; Hinokio, Y.; Tanizawa, Y.; Matsutani, A.; Satoh, J.; Oka, Y. Role of urotensin II gene in genetic susceptibility to Type 2 diabetes mellitus in Japanese subjects. Diabetologia (2003) 46, 972-976; and Langham, R. G.; Kelly, D. J.; Gow, R. M.; Zhang, Y.; Dowling, J. K.; Thomson, N. M.; Gilbert, R. E. Increased expression of urotensin II and urotensin II receptor in human diabetic nephropathy. American Journal Of Kidney Diseases: The Official Journal Of The National Kidney Foundation (2004) 44, 826-831).

The physiological role that GPR-14 (the urotensin II receptor) serves in the mammalian central nervous system is currently unknown. Important insights into the possible physiological effects mediated by this G-protein coupled receptor can be gained from an understanding of which cells in brain express this gene. Recently, the pattern of expression of the mRNA that encodes this receptor was reported. (Clark S D et al Brain Res. (2001) 923:120-7; Huitron-Resendiz et al Journal of Neuroscience (2005) 25:5465-5474. The GPR-14 gene is expressed in a discrete, extremely limited distribution within the mammalian central nervous system. The only brain regions which express this mRNA are the pedunculopontine tegmental nucleus (PPT), and the lateral dorsal tegmental nucleus (LDTG). These brain stem nuclei are the source of the ascending acetylcholine projection neurons in mammals, and as such are quite well studied, and have had a number of important physiological roles assigned to them. The expression of this receptor gene in just these cholinergic neurons provides for a novel mechanism by which these cell groups can be selectively modulated by small organic compounds targeted to GPR-14.

### SUMMARY OF THE INVENTION

The present investigators have identified a class of non-endogenous, low molecular weight non-peptide organic compounds that act as specific modulators of the urotensin II receptor.

Aspects of the present invention relate to a compound of Formula I, as defined herein, or salts or prodrugs thereof. The compounds may appear as mixtures of isomers or as separated and purified isomers. Other aspects of the present invention relate to a complex between the human urotensin II receptor and a compound of the invention and to a method of preparing a complex between a compound of the invention and human urotensin II receptor comprising combining said compound in an effective concentration with human urotensin II receptor.

The present inventors have demonstrated for the first time that compounds of the invention, namely compounds of Formula I, as defined herein, to be potent modulators of the human urotensin II receptor. Correspondingly, a further aspect of the invention relates to a use of compound of Formula I, salts thereof, or compositions comprising said compounds, for the preparation of a medicament for the treatment of diseases and disorders for which activation or modulation of the urotensin II receptor produces a beneficial response in said disease or disorder. The diseases and disorders are selected from the group consisting of those associated with CNS function, such as Parkinson's Disease, Alzheimer's Disease, depression, amylotrophic lateral sclerosis, muscular dystrophy, childhood spinal muscular atrophy, progressive spinal muscular atrophy and progressive bulbar palsy; OPCA; ADHD; schizophrenia; sleep disorders such as insomnia and narcolepsy; and autonomic dysfunctions such as Shy Drager syndrome. Alternatively, the diseases or disorders are selected from the group consisting of cardiovascular disorders such as hypertension; hypotensive states related to shock, sepsis, major surgery, congestive heart failure, and pulmonary disorders. Alternatively, the diseases or disorders are selected from ischemic conditions, renal disorders, urinary disorders such as incontinence, and tumor growth in cancer.

As stated, a variety of disease states have been suggested to be associated with either an altered functioning of the urotensin II receptor or to an imbalance of urotensin II. For example, alteration of urotensin II and signaling through its cognate receptor may be associated with, amongst other disease-states, both hypertension and hypotension. Accordingly, a further aspect of the invention relates to method of altering the vascular pressure in a mammal, comprising constricting or dilating vascular tissue in said mammal, said constricting or dilating being performed by the activation of urotensin receptor signaling, said activation being performed by the administration of an effective amount of a compound of Formula I. Similarly, the invention relates to methods of altering the heart rate in a mammal, comprising the modulation of urotensin receptor signaling, said modulation being performed by the administration of an effective amount compound of Formula I.

Moreover, a method of treating diseases or disorders in a mammal, said diseases or disorders being associated with an altered urotensin II receptor activity or imbalance in urotensin II level or activity compared to urotensin receptor activity or urotensin II levels or activity in a mammal not having said disease or disorder, comprising administering an effective amount of a compound of Formula I is within the scope of the present invention. Accordingly, the present invention further relates to a method of treating diseases for which modulation of the urotensin II receptor produces a physiologically beneficial response in said disease, such as those associated with CNS function and cardiovascular diseases.

The present investigators have found that, upon administration of compounds of Formula I, the locomotor activity of the animal is altered. Accordingly, the invention further relates to a method of altering the locomotor activity of a mammal, comprising administering to said mammal an effective amount of a compound of Formula I.

This alteration of locomotor function may indicate a CNS-mediated response of a compound of Formula I and CNS mediated function of the urotensin II receptor that suggests application in CNS therapeutic areas. Thus, a further aspect of the invention relates to the treatment of diseases and disorders associated with CNS function. Given, the distribution of the urotensin II receptor within cardiovascular tissue, a further aspect of the invention relates to the treatment of cardiovascular disorders.

Thus, in a first aspect, the present invention relates to a compound of Formula I, or salts or prodrugs thereof, complexed with a human urotensin II receptor,

An aspect the invention is related to a compound having the chemical structure of Formula I:

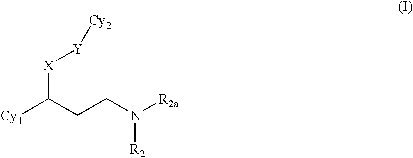

as a single isomer, a mixture of isomers, or a as a racemic mixture of isomers; as a solvate or a pharmaceutically acceptable salt thereof.

X can be selected from the group consisting of: C₁-C₄alkylene, C₁-C₄alkenylene, C₁-C₄alkynylene, —N(R₁)—, and —O—.

Y can be selected from the group consisting of: C₁-C₄alkylene, C₁-C₄alkenylene, C₁-C₄alkynylene, —C(═O)—, —C(═O)N(R₁)—, —S(O)₂—, —S(O)—, —S(O)₂N(R₁)—, —S(O)N(R₁)—, —N(R₁)—: —C(═O)O—, —C(═O)O—W—, —C(═O)W—, —C(═O)CH(OR₁)—, —C(═O)N(R₁)—, —C(═O)N(R₁)—W—, —S(O)₂—W—, —S(O)—W—, —S(O)₂N(R₁)—W—, —S(O)N(R₁)—W— and —N(R₁)—W—.

W can be selected from the group consisting of: C₁-C₄alkylene, C₁-C₄alkenylene, and C₁-C₄alkynylene.

R₁, R₁ₐ and R_{1b} can be each independently selected from the group consisting of: hydrogen, alkyl, alkenyl, alkynyl, cycloalkyl, cycloalkenyl, aryl, heteroaryl, and heteroalicyclyl.

Cy₁ and Cy₂ can be each independently selected from the group consisting of aryl and heteroaryl.

R₂ and R₂ₐ can be each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ can be taken together to form a C₂-C₁₀ heteroalicyclyl.

Z can be oxygen or sulfur.

In some embodiments, Cy₁ and Cy₂ can be each independently selected from the group consisting of:

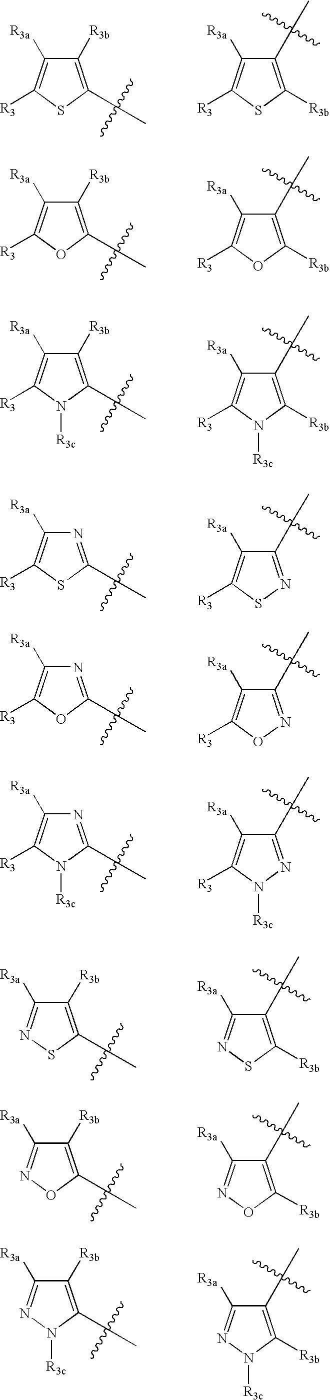

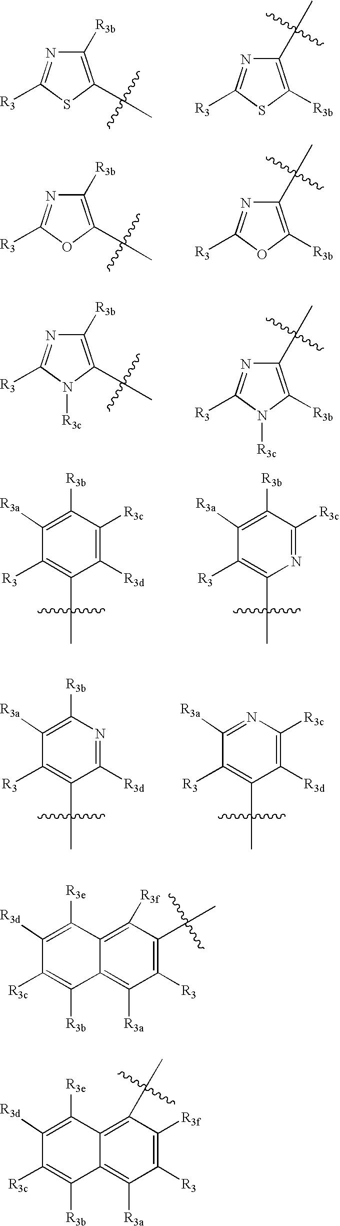

wherein R₃, R₃ₐ, R_{3b}, R_{3c}, R_{3d}, R₃ₑ and R_{3f} can be each independently selected from the group consisting of hydrogen, alkyl, alkenyl, alkynyl, cycloalkyl, cycloalkenyl, aryl, heteroaryl, heteroalicyclyl, halogen, hydroxyl, nitro, sulfenyl, sulfinyl, sulfonyl, haloalkyl, haloalkoxy, —CN, —C(=Z)R₁, —C(=Z)OR₁, C(=Z)NR₁R₁ₐ, —C(R₁)═NR₁ₐ, NR₁R₁ₐ, N═CR₁R₁ₐ, —N(R₁)—C(=Z)R₁ₐ, —N(R₁)—C(=Z)NR₁ₐR_{1b}, —S(O)NR₁R₁ₐ, —S(O)₂NR₁R₁ₐ, —N(R₁)—S(═O)R₁ₐ, —N(R₁)—S(═O)₂R₁ₐ, —OR₁, —SR₁, and —OC(=Z)R₁; or two R groups selected from the group consisting of R₃, R₃ₐ, R_{3b}, R_{3c}, R_{3d}, R₃ₑ, and R_{3f} are covalently bonded to adjacent atoms, then they can be taken together, as defined herein, to form a cycloalkyl, aryl, heteroaryl or heteroalicyclyl group.

In some embodiments, the compound of Formula I can have X is —N(R₁)—; Y is selected from the group consisting of C₁-C₄alkylene, C₁-C₄alkenylene, C₁-C₄alkynylene, —C(═O)—, —C(═O)N(R═)—, —S(O)₂—, —S(O)—, —S(O)₂N(R₁)—, —S(O)N(R₁)—, —N(R₁)—: —C(═O)O—, —C(═O)O—W—, —C(═O)W—, —C(═O)CH(OR₁)—, —C(═O)N(R₁)—, —C(═O)N(R₁)—W—, —S(O)₂—W—, —S(O)—W—, —S(O)₂N(R₁)—W—, —S(O)N(R₁)—W— and —N(R₁)—W—; Cy₁ and Cy₂ are each independently selected from the group consisting of aryl and heteroaryl; and R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ can be taken together to form a C₂-C₁₀ heteroalicyclyl.

In other embodiments, the compound of Formula I can have X is —N(R₁)—; Y is —C(═O)—; Cy₁ and Cy₂ are each independently selected from the group consisting of aryl and heteroaryl; and R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ can be taken together to form a C₂-C₁₀ heteroalicyclyl.

In still other embodiments, the compound of Formula I can have X is —N(R₁)—; Y is —C(═O)—; Cy₁ and Cy₂ are aryls; and R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ may be taken together to form a C₂-C₁₀ heteroalicyclyl.

In yet sill other embodiments, the compound of Formula I can have X is —N(R₁)—; Y is —C(═O)—; Cy₁ and Cy₂ are p-substituted aryls; and R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ may be taken together to form a C₂-C₁₀ heteroalicyclyl.

In some embodiments, the compound of Formula I can have X is —N(R₁)—; Y is —C(═O)—; Cy₁ is a p-substituted aryl substituted with a halogen; Cy₂ is a p-substituted aryl substituted with an aryl; and R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ may be taken together to form a C₂-C₁₀ heteroalicyclyl.

In other embodiments, the compound of Formula I can have X is —N(R₁)—; Y is —C(═O)—; Cy₁ is a p-substituted aryl substituted with a halogen; Cy₂ is a p-substituted aryl substituted with an aryl; and R₂ and R₂, are alkyl groups.

In some embodiments, the compound of Formula I can be a polymorph, ester, metabolite or prodrug.

Another aspect of this invention is a pharmaceutical composition comprising a pharmaceutically acceptable amount of a compound of Formula I.

An aspect of this invention is a method of treating or preventing disorders selected from the group consisting of a CNS disorder, depression, a sleep disorder, an autonomic dysfunction a cardiovascular disorder, a renal disorder, incontinence, and cancer, tumor growth, and diabetes comprising identifying a subject in need of said treating or preventing; and administering to the subject a pharmaceutically effective amount of a compound of Formula I. In one embodiment, the CNS disorder can be selected from group consisting of Parkinson's Disease, Alzheimer's Disease, amylotrophic lateral sclerosis, muscular dystrophy, childhood spinal muscular atrophy, progressive spinal muscular atrophy and progressive bulbar palsy, OPCA, ADHD, and schizophrenia. In another embodiment, the cardiovascular disorder can be selected from the group consisting of heart failure, atherosclerosis, hypertension and hypotensive states related to shock, sepsis, major surgery, congestive heart, and pulmonary disorders. In still another embodiment, the sleep disorder can be selected from the group consisting of insomnia and narcolepsy. In yet still another embodiment, the autonomic dysfunction can be Shy Drager syndrome.

Yet another aspect of this invention is a compound that can be selected from the group consisting of:
[3-(4-Chlorophenyl)-3-(4-methylbenzyloxypropyl]-N,N-dimethyl amine (3a);
[3-(4-Chlorophenyl)-3-(2-methoxybenzyloxypropyl]-N,N-dimethyl amine (3b);
1-(4-Chlorophenyl)-3-dimethylamino-propyl 2-methyl-benzoate HCl (4a);
1-(4-Chlorophenyl)-3-dimethylamino-propyl 2-ethyl-benzoate HCl (4b);
1-(4-Chlorophenyl)-3-dimethylamino-propyl 4-methyl-benzoate HCl (4c);
1-(4-Chlorophenyl)-3-dimethylamino-propyl 2,3-dimethyl-benzoate HCl (4d);
1-(4-Chlorophenyl)-3-dimethylamino-propyl 3-methoxy-2-methyl-benzoate HCl (4e);
1-(4-Chlorophenyl)-3-dimethylamino-propyl 3-chloro-2-methyl-benzoate HCl (4f);
1-(4-Chlorophenyl)-3-dimethylamino-propyl 4-bromo-2-methyl-benzoate HCl (4g);
1-(4-Chlorophenyl)-3-dimethylamino-propyl 2,5-dimethyl-benzoate HCl (4h);
1-(4-Chlorophenyl)-3-dimethylamino-propyl 2,4,5-trimethyl-benzoate HCl (4i);
1-(4-Chlorophenyl)-3-dimethylamino-propyl 3-methyl-thiophene-2-carboxylate HCl (4j);
1-(4-Chlorophenyl)-3-dimethylamino-propyl naphthalene-1-carboxylate HCl (4k);
1-(4-Chlorophenyl)-3-dimethylamino-propyl naphthalene-2-carboxylate HCl (4l);
1-(4-Chlorophenyl)-3-dimethylamino-propyl 4-phenyl-benzoate HCl (4m);
1-(4-Chlorophenyl)-3-dimethylamino-propyl 1-methyl-indole-2-carboxylate HCl (4n);
1-(4-Chlorophenyl)-3-dimethylamino-propyl 1-methyl-indole-3-carboxylate HCl (4o);
1-(4-Chlorophenyl)-3-dimethylamino-propyl 4-methoxy-benzoate oxalate (4p);
1-(4-Chlorophenyl)-3-dimethylamino-propyl 4-trifluoromethyl-benzoate oxalate (4q);
N-[1-(4-Chlorophenyl)-3-dimethylamino-propyl]-benzamide HCl (5a);
N-[1-(4-Chlorophenyl)-3-dimethylamino-propyl]-2-methyl-benzamide oxalate (5b);
N-[1-(4-Chlorophenyl)-3-dimethylamino-propyl]-2-ethyl-benzamide oxalate (5c);
N-[1-(4-Chlorophenyl)-3-dimethylaminopropyl]-4-methoxy-benzamide oxalate (5d);
N-[1-(4-Chlorophenyl)-3-dimethylaminopropyl]-4-dimethylamino-benzamide oxalate (5e);
N-[1-(4-Chlorophenyl)-3-dimethylamino-propyl]-2,3-dimethyl-benzamide oxalate (5f);
N-[1-(4-Chlorophenyl)-3-dimethylamino-propyl]-3-methoxy-2-methyl-benzamide oxalate (5g);
N-[1-(4-Chlorophenyl)-3-dimethylamino-propyl]-3-chloro-2-methyl-benzamide oxalate (5h);
N-[1-(4-Chlorophenyl)-3-dimethylamino-propyl]-2,4-dimethyl-benzamide oxalate (5i);
N-[1-(4-Chlorophenyl)-3-dimethylamino-propyl]-2,5-dimethyl-benzamide oxalate (5j);
N-[1-(4-Chlorophenyl)-3-dimethylamino-propyl]-6-chloro-2-methyl-benzamide oxalate (5k);
N-(1-(4-chlorophenyl)-3-(dimethylamino)propyl)benzo[d][1,3]dioxole-5-carboxamide oxalate (5l);
N-[1-(4-Chlorophenyl)-3-dimethylaminopropyl]-2,4,5-trimethyl-benzamide oxalate (5m);
N-[1-(4-Chlorophenyl)-3-dimethylamino-propyl]-2-naphthyl-carboxamide oxalate (5n);
N-[1-(4-Chlorophenyl)-3-dimethylamino-propyl]-4-phenyl-benzamide oxalate (5o);
(−)-N-[1-(4-Chlorophenyl)-3-dimethylamino-propyl]-4-phenyl-benzamide oxalate ((−)-5o);
(+)-N-[1-(4-Chlorophenyl)-3-dimethylamino-propyl]-4-phenyl-benzamide oxalate ((+)-5o);
N-[1-(4-Chlorophenyl)-3-dimethylaminopropyl]-4-phenoxy-benzamide oxalate (5p);
N-[1-(4-Chlorophenyl)-3-dimethylaminopropyl]-4-trifluoromethyl-benzamide oxalate (5q);
N-[1-(4-Chlorophenyl)-3-dimethylaminopropyl]-2-phenyl-acetamide oxalate (5r);
N-[1-(4-Chlorophenyl)-3-dimethylaminopropyl]-(R)-2-methoxy-2-phenyl acetamide oxalate (5s);
(+)-N-[1-(4-Chlorophenyl)-3-dimethylaminopropyl]-(R)-2-methoxy-2-phenyl-acetamide oxalate ((+)-5s);
(−)-N-[1-(4-Chlorophenyl)-3-dimethylaminopropyl]-(R)-2-methoxy-2-phenyl-acetamide oxalate ((−)-5s);
N-[1-(4-Chlorophenyl)-3-dimethylaminopropyl]-4-methyl-benzenesulfonamide oxalate (6a);
N-[1-(4-Chlorophenyl)-3-dimethylaminopropyl]-4-phenyl-benzenesulfonamide oxalate (6b);
N-[1-(4-Chlorophenyl)-3-dimethylaminopropyl]-2-naphthyl-benzenesulfonamide oxalate (6c);
N-[1-(4-Chlorophenyl)-3-dimethylaminopropyloxycarbonyl]-2-methylphenyl-amine (7a);
N-[1-(4-Chlorophenyl)-3-dimethylaminopropyloxycarbonyl]-3-methoxyphenyl-amine (7b);
N-[1-(4-Chlorophenyl-3-dimethylaminopropyloxycarbonyl]-4-tert-butylphenyl-amine (7c);
N-[1-(4-Chlorophenyl)-3-dimethylaminopropyloxycarbonyl]-4-phenoxyphenyl-amine (7d);
N-[1-(4-Chlorophenyl)-3-dimethylaminopropyloxycarbonyl]benzyl-amine (7e);
N-[1-(4-Chlorophenyl)-3-dimethylaminopropyloxycarbonyl]-4-phenyl-amine (7f);
N-[1-(4-Chlorophenyl)-3-dimethylaminopropyloxycarbonyl]-2-naphthyl-amine (7g);
N-[1-(4-Chlorophenyl)-3-dimethylaminopropyloxycarbonyl]-4-methoxyphenyl-amine (7h);
N-[1-(4-Chlorophenyl)-3-dimethylaminopropyloxycarbonyl]-4-trifluoromethylphenyl-amine (7i);
1-[1-(4-Chlorophenyl)-3-dimethylaminopropyl]-3-(2-methylphenyl)carbamide oxalate (8a);
1-[1-(4-Chlorophenyl)-3-dimethylaminopropyl]-3-(4-tert-butylphenyl)carbamide oxalate (8c);
1-[1-(4-Chlorophenyl)-3-dimethylaminopropyl]-3-(4-phenoxyphenyl carbamide oxalate (8d);
1-[1-(4-Chlorophenyl)-3-dimethylaminopropyl]-3-benzyl-carbamide oxalate (8e);
1-[1-(4-Chlorophenyl)-3-dimethylaminopropyl]-3-(4-phenylphenyl)-carbamide oxalate (8f);
1-[1-(4-Chlorophenyl)-3-dimethylaminopropyl]-3-(2-naphthyl)-carbamide oxalate (8g);
1-[1-(4-Chlorophenyl)-3-dimethylaminopropyl]-3-(4-methoxyphenyl carbamide oxalate (8h);
1-[1-(4-Chlorophenyl)-3-dimethylaminopropyl]-3-(4-trifluoromethylphenyl)-carbamide oxalate (8i);
N-[1-(4-Chlorophenyl)-3-dimethylamino-propyl]-2-phenylacetamide HCl (A1);
N-[1-(4-Chlorophenyl)-3-dimethylamino-propyl]-2-(4-trifluoromethylphenyl)acetamide HCl (A2);
N-[1-(4-Chlorophenyl)-3-dimethylamino-propyl]-2-(4-methoxyphenyl)acetamide HCl (A3);
N-[1-(4-Chlorophenyl)-3-dimethylamino-propyl]-3-phenyl-propionamide HCl (A4);
N-[1-(4-Chlorophenyl)-3-dimethylamino-propyl]-3-(4-trifluoromethylphenyl) propionamide HCl (A5);
N-[1-(4-Chlorophenyl)-3-dimethylamino-propyl]-3-(4-methoxyphenyl)propanamide HCl (A6);
N-[1-(4-Chlorophenyl)-3-dimethylamino-propyl]-3-cinnamic amide HCl (A7);
N-[1-(4-Chlorophenyl)-3-dimethylamino-propyl]-4-trifluoromethyl-cinnamic amide HCl (A8);
N-[1-(4-Chlorophenyl)-3-dimethylamino-propyl]-4-methoxy-cinnamic amide HCl (A9);
N-[1-(4-Chlorophenyl)-3-dimethylamino-propyl]-3-phenylpropiolic amide HCl (A10);
N-[3-Dimethylamino-1-(4-methylphenyl)propyl]-2-phenylacetamide HCl (B1);
N-[3-Dimethylamino-1-(4-methylphenyl)propyl]-2-(4-trifluoromethylphenyl)acetamide HCl (B2);
N-[3-Dimethylamino-1-(4-methylphenyl)propyl]-2-(4-methoxyphenyl)acetamide HCl (B3);
N-[3-Dimethylamino-1-(4-methylphenyl)propyl]-3-phenylpropionamide HCl (B4);
N-[3-Dimethylamino-1-(4-methylphenyl)propyl]-3-(4-trifluoromethylphenyl) propionamide HCl (B5);
N-[3-Dimethylamino-1-(4-methylphenyl)propyl]-3-(4-methoxyphenyl)propionamide HCl (B6);
N-[3-Dimethylamino-1-(4-methylphenyl)propyl]cinnamic amide (B7);
N-[3-Dimethylamino-1-(4-methylphenyl)propyl]-4-trifluoromethyl-cinnamic amide HCl (B8);
N-[3-Dimethylamino-1-(4-methylphenyl)propyl]-4-methoxy-cinnamic amide HCl (B9);
N-[3-Dimethylamino-1-(4-methylphenyl)propyl]-3-phenylpropiolic amide (B10);
N-[3-Dimethylamino-1-(2-naphthyl)propyl]-2-phenylacetamide HCl (C1);
N-[3-Dimethylamino-1-(2-naphthyl)propyl]-2-(4-trifluoromethylphenyl)acetamide HCl (C2);
N-[3-Dimethylamino-1-(2-naphthyl)propyl]-2-(4-methoxyphenyl)acetamide HCl (C3);
N-[3-Dimethylamino-1-(2-naphthyl)propyl]-3-phenyl-propionamide HCl (C4);
N-[3-Dimethylamino-1-(2-naphthyl)propyl]-2-(4-trifluoromethylphenyl)propionamide HCl (C5);
N-[3-Dimethylamino-1-(2-naphthyl)propyl]-3-(4-methoxyphenyl-propion amide HCl (C6);
N-[3-Dimethylamino-1-(2-naphthyl)propyl]-3-cinnamic amide HCl (C7);
N-[3-Dimethylamino-1-(2-naphthyl)propyl]-4-trifluoromethyl-cinnamic amide HCl (C8);
N-[3-Dimethylamino-1-(2-naphthyl)propyl]-4-methoxy-cinnamic amide HCl (C9); and
N-[3-Dimethylamino-1-(2-naphthyl)propyl]-3-phenylpropiolic amide HCl (C10).

Another aspect of this invention is a method of identifying a compound which is an agonist, inverse agonist, or antagonist of the urotensin receptor, the method comprising contacting a urotensin receptor with at least one test compound of Formula I; and determining any increase or decrease in activity level of said urotensin receptor.

### BRIEF DESCRIPTION OF THE DRAWINGS

FIG. 1 is a bar graph of the urotensin receptor agonist potencies of the synthesized amides divided into families.

FIG. 2 is a graph of the urotensin receptor activity of A1, A4, A7 and A10 in the functional cell based R-SAT assay.

FIG. 3 is a graph of the scatter plot of the correlation between efficacy and pEC₅₀ values for aliphatic [A1-C6] (diamonds) and conjugated derivatives [A7-C10] (squares).

### DETAILED DESCRIPTION OF THE PREFERRED EMBODIMENT

Definitions

Unless defined otherwise, all technical and scientific terms used herein have the same meaning as is commonly understood by one of ordinary skill in the art to which this invention belongs. All patent, applications, published applications and other publications are incorporated by reference in their entirety. In the event that there are a plurality of definitions for a term herein, those in this section prevail unless stated otherwise

As used herein, any “R” group(s) such as, without limitation, R₁, R₁ₐ, R_{1b}, R₂, R₂ₐ, R₃, R₃ₐ, R_{3b}, R_{3c}, R_{3d}, R₃ₑ, and R_{3f} is(are) independently selected from the group consisting of hydrogen, alkyl, alkenyl, alkynyl, cycloalkyl, cycloalkenyl, aryl, heteroaryl, heteroalicyclyl, halogen, hydroxyl, nitro, sulfenyl, sulfinyl, sulfonyl, haloalkyl, haloalkoxy, aralkyl, —CN, —C(=Z)R₁, —C(=Z)OR₁, —C(=Z)NR₁R₁ₐ, —C(R₁)═NR₁ₐ, —NR₁R₁ₐ, —N═CR₁R₁ₐ, —N(R₁)—C(=Z)R₁ₐ, N(R₁)—C(=Z)NR₁ₐR_{1b}, —S(O)NR₁R₁ₐ, —S(O)₂NR₁R₁ₐ, —N(R₁)—S(═O)R₁ₐ, —N(R₁)—S(═O)₂R₁ₐ, —OR₁, —SR₁, and —OC(=Z)R₁, as these groups are defined herein. If two “R” groups are covalently bonded to the same atom or to adjacent atoms, then they may be “taken together” as defined herein to form a cycloalkyl, aryl, heteroaryl or heteroalicyclyl group. For example, without limitation, if R₁ₐ and R_{1b} of an NR₁ₐ R_{1b} group are indicated to be “taken together,” it means that they are covalently bonded to one another at their terminal atoms to form a ring: 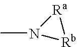
An R group of this invention may be substituted or unsubstituted.

As used herein, “IC₅₀” refers to an amount, concentration of dosage of a particular test compound that achieves a 50% inhibition of a maximal response, such as modulation of GPCR, including Urotensin II receptor, activity, in an assay that measures such response in an assay that measures such response for example but not limited to R-SAT™ described herein.

As used herein, “EC₅₀” refers to an dosage, concentration or amount of a particular test compound that elicits a dose-dependent response at 50% of maximal expression of a particular response that is induced, provoked or potentiated by the particular test compound, in an assay that measures such response for example but not limited to R-SAT™ described herein.

Whenever a group of this invention is described as being “optionally substituted” that group may be unsubstituted or substituted with one or more of the indicated substituents. Likewise, when a group is described as being “unsubstituted or substituted” if substituted, the substituent may be selected from the same group of substituents.

As used herein, “Cₘ to Cₙ” in which “m” and “n” are integers refers to the number of carbon atoms in an alkyl, alkenyl or alkynyl group or the number of carbon atoms in the ring of a cycloalkyl or cycloalkenyl group. That is, the alkyl, alkenyl, alkynyl, ring of the cycloalkyl or ring of the cycloalkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)— and (CH₃)₃CH—. If no “m” and “n” are designated with regard to an alkyl, alkenyl, alkynyl, cycloalkyl or cycloalkenyl group, the broadest range described in these definitions is to be assumed.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group of this invention may be substituted or unsubstituted. When substituted, hydrogen atoms are replaced by substituent group(s) that is(are) one or more group(s) independently selected from alkyl, cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR₁ₐR_{1b} and protected amino.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. The heteroaryl group may be optionally fused to a benzene ring. Examples of heteroaryl rings include, but are not limited to, furan, thiophene, phthalazinone, pyrrole, oxazole, thiazole, imidazole, pyrazole, isoxazole, isothiazole, triazole, thiadiazole, pyran, pyridine, pyridazine, pyrimidine, pyrazine and triazine. A heteroaryl group of this invention may be substituted or unsubstituted. When substituted, hydrogen atoms are replaced by substituent group(s) that is(are) one or more group(s) independently selected from alkyl, cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbarnyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR₁ₐR_{1b} and protected amino

As used herein, “alkyl” refers to a straight or branched hydrocarbon chain fully saturated (no double or triple bonds) hydrocarbon group. An alkyl group of this invention may comprise from 1 to 20 carbon atoms. An alkyl group herein may also be of medium size having 1 to 10 carbon atoms. It is presently preferred that an alkyl group of this invention be a lower alkyl having 1 to 4 carbon atoms. Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this invention may be substituted or unsubstituted. When substituted, hydrogen atoms are replaced by substituent group(s) that is(are) one or more group(s) independently selected from cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR₁ₐR_{1b} and protected amino.

“Aralkyl groups” are aryl groups connected, as substituents, via an alkylene group. The aryl and alkylene group of an aralkyl group may be substituted or unsubstituted. Examples includes but are not limited to benzyl, substituted benzyl, 2-phenylethyl, 3-phenylpropyl, naphtylalkyl.

“Heteroaralkyl groups” are understood as heteroaryl groups connected, as substituents, via an alkylene group. The heteroaryl and alkylene group of a heteroaralkyl group may be substituted or unsubstituted. Examples includes but are not limited to 2-thienylmethyl, 3-thienylmethyl, furylmethyl, thienylethyl, pyrrolylalkyl, pyridylalkyl, isoxazollylalkyl, imidazolylalkyl, and their substituted as well as benzo-fused analogs.

As used herein, “alkoxy” and “alkylthio” refers to RO— and RS—, in which R is an unsubstituted or substituted alkyl, including a lower alkyl. Examples include but are not limited to methoxy, ethoxy, n-propoxy, 1-methylethoxy (isopropoxy), n-butoxy, iso-butoxy, sec-butoxy, tert-butoxy, amoxy, tert-amoxy and the like.

As used herein, “aryloxy” and “arylthio” refers to RO— and RS—, in which R is an unsubstituted or substituted aryl, such as but not limited to phenyl.

As used herein, “alkenyl” refers to an alkyl group that contains in the straight or branched hydrocarbon chain one or more double bonds. An alkenyl group may be unsubstituted or substituted. When substituted, the substituent(s) may be selected from the same groups disclosed above with regard to alkyl group substitution.

As used herein, “alkylidene” refers to a divalent group, such as ═CR′R″, which is attached to one carbon of another group, forming a double bond, Alkylidene groups include, but are not limited to, methylidene (═CH₂) and ethylidene (═CHCH₃). As used herein, “arylalkylidene” refers to an group to an alkylidene group in which either R′ and R″ is an aryl group.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution.

The term “alkylene” refers to an alkyl group, as defined here, which is a biradical and is connected to two other moieties. An alkylene group of this invention may be unsubstituted or substituted. Thus, methylene (—CH₂—), ethylene (—CH₂CH₂—), propylene (—CH₂CH₂CH₂—), isopropylene (—CH₂—CH(CH₃)—), and isobutylene (—CH₂—CH(CH₃)—CH₂—) are examples, without limitation, of an alkylene group.

The term “alkenylene” refers to an alkylene group, as defined here, that contains in the straight or branched hydrocarbon chain one or more double bonds. The group is a bivalent radical derived by removing a hydrogen atom from each of the terminal carbon atoms. If only one double bond is present in the hydrocarbon chain is it represented by the formula —(CₙH₂ₙ₋₂)—. An alkenylene group of this invention may be unsubstituted or substituted. When substituted, the substituent(s) may be selected from the same groups disclosed above with regard to alkyl group substitution. Alkenylene groups include, but are not limited to, propenylene —HC═C═CH— and vinylene (ethenylene) —HC═CH—.

The term “alkynylene” refers to an alkylene group, as defined here, that contains in the straight or branched hydrocarbon chain one or more triple bonds. The group is a bivalent radical derived by removing two hydrogen atoms from each of the terminal carbon atoms. If only one triple bond is present in the hydrocarbon chain is it represented by the formula —(CₙH₂ₙ₋₄)—. An alkynylene group of this invention may be unsubstituted or substituted.

As used herein, “acyl” refers to an “RC(═O)—” group with R as defined above.

As used herein, “cycloalkyl” refers to a completely saturated (no double bonds) mono- or multi-cyclic hydrocarbon ring system. Cycloalkyl groups of this invention may range from C₃ to C₁₀. In other embodiments it may range from C₃ to C₆. A cycloalkyl group may be unsubstituted or substituted. If substituted, the substituent(s) may be selected from those indicated above with regard to substitution of an alkyl group.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of this invention may be unsubstituted or substituted. When substituted, the substituent(s) may be selected from the groups disclosed above with regard to alkyl group substitution.

As used herein, “heteroalicyclic” or “heteroalicyclyl” refers to a stable 3- to 18-membered ring which consists of carbon atoms and from one to five heteroatoms selected from the group consisting of nitrogen, oxygen and sulfur. For the purpose of this invention, the “heteroalicyclic” or “heteroalicyclyl” may be monocyclic, bicyclic, tricyclic, or tetracyclic ring system, which may include fused or bridged ring systems; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system in the rings. Heteroalicyclyl groups of this invention may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl N-Oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone.

The ring systems of the cycloalkyl, heteroalicyclic (heteroalicyclyl) and cycloalkenyl groups may be composed of one ring or two or more rings which may be joined together in a fused, bridged or spiro-connected fashion.

As used herein, “halo” or “halogen” refers to F (fluoro), Cl (chloro), Br (bromo) or I (iodo).

As used herein, “haloalkyl” refers to an alkyl group in which one or more of the hydrogen atoms are replaced by halogen. Such groups include but are not limited to, chloromethyl, fluoromethyl, difluoromethyl, trifluoromethyl and 1-chloro-2-fluoromethyl, 2-fluoroisobutyl.

As used herein, “haloalkoxy” refers to RO-group in which R is a haloalkyl group. Such groups include but are not limited to, chloromethoxy, fluoromethoxy, difluoromethoxy, trifluoromethoxy and 1-chloro-2-fluoromethoxy, 2-fluoroisobutyoxy.

An “O-carboxy” group refers to a “RC(═O)O—” group with R as defined above.

A “C-carboxy” group refers to a “—C(═O)R” group with R as defined above.

An “acetyl” group refers to a CH₃C(═O)— group.

A “trihalomethanesulfonyl” group refers to an “X₃CSO₂—” group wherein X is a halogen.

A “cyano” group refers to a “—CN” group.

An “isocyanato” group refers to an “—NCO” group.

A “thiocyanato” group refers to a “—CNS” group.

An “isothiocyanato” group refers to an “—NCS” group.

A “sulfinyl” group refers to an “—S(═O)—R” group with R as defined above.

A “sulfonyl” group refers to an “SO₂R” group with R as defined above.

An “S-sulfonamido” group refers to a “—SO₂NR₁ₐR_{1b}” group with R₁ₐ and R_{1b} as defined above.

An “N-sulfonamido” group refers to a “RSO₂N(R₁ₐ)—” group with R and R₁ₐ as defined above.

A “trihalomethanesulfonamido” group refers to an “X₃CSO₂N(R)—” group with X as halogen and R as defined above.

An “O-carbarnyl” group refers to a “—OC(═O)NR₁ₐR_{1b}” group with R₁ₐ and R_{1b} as defined above.

An “N-carbarnyl” group refers to an “ROC(═O)NR₁ₐ—” group with R₁ₐ and R as defined above.

An “O-thiocarbamyl” group refers to a “—OC(═S)—NR₁ₐR_{1b}” group with R₁ₐ and R_{1b} as defined above.

An “N-thiocarbamyl” group refers to an “ROC(═S)NR₁ₐ—” group with R₁ₐ and R as defined above.

A “C-amido” group refers to a “—C(═O)NR₁ₐR_{1b}” group with R₁ₐ and R_{1b} as defined above.

An “N-amido” group refers to a “RC(═O)NR₁ₐ—” group with R and R₁ₐ as defined above.

As used herein, an “ester” refers to a “—C(═O)OR” group with R as defined above.

As used herein, an “amide” refers to a “—C(═O)NR₁ₐR_{1b}” group with R₁ₐ and R_{1b} as defined above.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999).

Where the number of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxygroups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As employed herein, the following terms have their accepted meaning in the chemical literature.

AcOH acetic acid

anhyd anhydrous

CDI 1,1′-carbonyldiimidazole

DCM dichloromethane

DMF N,N-dimethylformamide

DMSO dimethyl sulfoxide

Et₂O diethyl ether

EtOAc ethyl acetate

EtOH Ethanol

MeOH Methanol

NH₄OAc ammonium acetate

Pd/C palladium on activated carbon

It is understood that, in any compound of this invention having one or more chiral centers, if an absolute stereochemistry is not expressly indicated, then each center may independently be of R-configuration or S-configuration or a mixture thereof. Thus, the compounds provided herein may be enatiomerically pure or be stereoisomeric or dia stereomeric mixtures. In addition it is understood that, in any compound of this invention having one or more double bond(s) generating geometrical isomers that can be defined as E or Z each double bond may independently be E or Z a mixture thereof. Likewise, all tautomeric forms are also intended to be included.

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methanesulfonates, ethanesulfonates, p-toluenesulfonates and salicylates.

Pharmaceutically acceptable solvates and hydrates are complexes of a compound with one or more solvent of water molecules, or 1 to about 100, or 1 to about 10, or one to about 2, 3 or 4, solvent or water molecules.

As used herein, a “prodrug” refers to a compound that may not be pharmaceutically active but that is converted into an active drug upon in vivo administration. The prodrug may be designed to alter the metabolic stability or the transport characteristics of a drug, to mask side effects or toxicity, to improve the flavor of a drug or to alter other characteristics or properties of a drug. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to release the active parent compound. By virtue of knowledge of pharmacodynamic processes and drug metabolism in vivo, those skilled in the art, once a pharmaceutically active compound is known, can design prodrugs of the compound (see, e.g. Nogrady (1985) Medicinal Chemistry A Biochemical Approach, Oxford University Press, New York, pages 388-392)

As used herein, the term “complement” refers to a oligonucleotide or polynucleotide that hybridizes by base-pairing, adenine to tyrosine and guanine to cytosine, to another oligonucleotide. The hybridized oligonucleotides are then said to be complementary.

As used herein, to “modulate” the activity of UII means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all, even in the presence of a natural binding partner. A natural binding partner is an endogenous molecule that is an agonist for the receptor.

As used herein, to “detect” changes in the activity of UII or of a UII sub-type refers to the process of analyzing the result of an experiment using whatever analytical techniques are best suited to the particular situation. In some cases simple visual observation may suffice, in other cases the use of a microscope, visual or UV light analyzer or specific protein assays may be required. The proper selection of analytical tools and techniques to detect changes in the activity of UII or a UII sub-type are well-known to those skilled in the art.

An “agonist” is defined as a compound that increases the basal activity of a receptor (i.e. signal transduction mediated by the receptor).

As used herein, “partial agonist” refers to a compound that has an affinity for a receptor but, unlike an agonist, when bound to the receptor it elicits only a fractional degree of the pharmacological response normally associated with the receptor even if a large number of receptors are occupied by the compound.

An “inverse agonist” is defined as a compound which reduces, or suppresses the basal activity of a receptor, such that the compound is not technically an antagonist but, rather, is an agonist with negative intrinsic activity.

As used herein, “antagonist” refers to a compound that binds to a receptor to form a complex that does not give rise to any response, as if the receptor were unoccupied. An antagonist attenuates the action of an agonist on a receptor. An antagonist may bind reversibly or irreversibly, effectively eliminating the activity of the receptor permanently or at least until the antagonist is metabolized or dissociates or is otherwise removed by a physical or biological process.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by an M.D. or a D.V.M. to attempt to cure, or at least ameliorate the effects of, a particular disease or disorder or to prevent the disease or disorder from occurring in the first place.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Synthesis

General synthetic routes to the compounds of this invention are shown in Schemes 1 and 2. The routes shown are illustrative only and are not intended, nor are they to be construed, to limit the scope of this invention in any manner whatsoever. Those skilled in the art will be able to recognize modifications of the disclosed synthesis and to devise alternate routes based on the disclosures herein; all such modifications and alternate routes are within the scope of this invention.

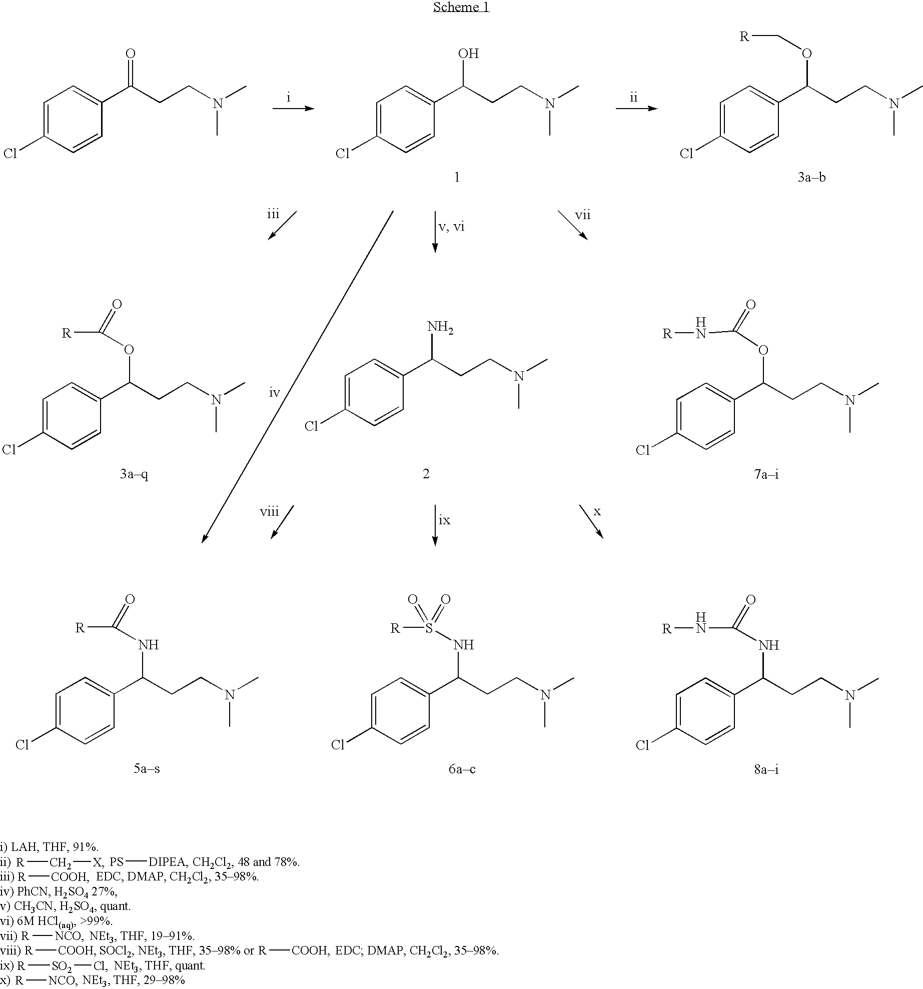

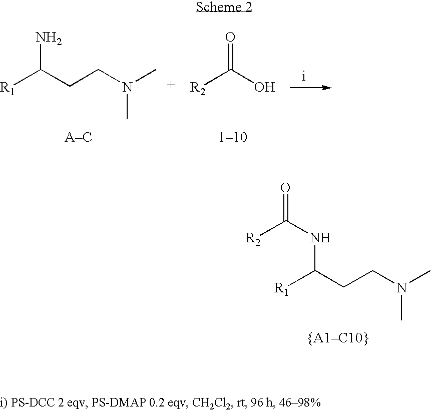

U.S. Pat. No. 4,564,641 discloses 2-phenyl-2-(2-phenethyl)-4-dialkylaminobutonoic acids as starting materials for the preparation of 1-oxo-2-phenyl-2-(2-allylaminoethyl)-1,2,3,4,-tetrahydronaphthalenes, compounds useful for treating depression. U.S. Pat. No. 3,880,885 discloses benzamides as starting materials for the preparation of tertiary aminoethyl isochromans and isocoumarins, compounds useful as antihypertensive or diuretic agents.

As stated, one aspect of the present invention relates to the use of a compound selected from the group comprising a compound of Formula I for the preparation of a medicament for the treatment of diseases and disorders for which activation or modulation of the urotensin II receptor produces a physiologically beneficial response in a given disorder.

A body of literature regarding the role of the pontine cholinergic nuclei and the modulation of cognitive processes has emerged in the last few years. Both basal forebrain and pontine cholinergic cell groups are known to control the activity of the hippocampal and cortical circuits that are critical for human attention, memory, and cognition As such, the selective modulation of the activity of the PPT and LDTG nuclei present a novel pharmacological means to affect cognition and memory. Potential Disease States and Therapeutic Indications, Alzheimer's Disease and related dementias, schizophrenia and related psychoses.

In light of the distribution of the urotensin II receptor within the central nervous system and within cardiovascular tissue, it is anticipated that the compounds of Formula I will be useful as medicaments to treat an array of neurodegenerative, neuropsychiatric, neurological and cardiovascular disorders. Accordingly, a further aspect of the invention relates to the use of compound of Formula I for the preparation of a medicament for the treatment of diseases and disorders in a mammal selected from the group consisting of diseases and disorders associated with CNS function, such as Parkinson's Disease, Alzheimer's Disease, amylotrophic lateral sclerosis, muscular dystrophy, childhood spinal muscular atrophy, progressive spinal muscular atrophy and progressive bulbar palsy, OPCA, ADHD, schizophrenia, sleep disorders such as insomnia, and autonomic dysfunctions such as Shy Drager syndrome. In addition, compounds of Formula I may be useful as medicaments to treat cardiovascular disorders such as hypertension; hypotensive states related to shock, sepsis, major surgery and congestive heart failure.

The present invention further relates to a method of altering the locomotor activity of a mammal, comprising administering to said mammal an effective amount of a compound of Formula I.

The decrease in locomotor activity and expression of urotensin II receptor in the brainstem are consistent with action of the compounds of Formula I on the CNS to alter sleep/wake patterns. The PPT and LDTG send ascending projections to the thalamus that are critical mediators of sleep and wakefulness in humans. During the sleep state, thalamocortical activity is dominated by rhythmic oscillations that are abolished during the transition to wakefulness, resulting in a significant increase in neuronal responsiveness. The cholinergic cells groups are one of the primary mediators of this transition, where neuronal activity of the PPT and LDTG neurons increase with wakefulness. (Huitron-Resendiz et al., Journal of Neuroscience (2005) 25:5465-5474.) Therefore, modulators of GPR-14 which can increase the activity of these cells may increase wakefulness in humans, while those that decrease the activity of these neurons may induce sleep. Consistent with these observations are the potential clinical use of modulators of GPR-14 as CNS stimulants and sleep promoting CNS depressants (both perhaps without the addictive and physical dependency properties that limit the use of current agents).

Thus, potential disease states and therapeutic indications for which compounds of Formula I may be connected to include narcolepsy, non-addictive CNS Stimulant, ADHD and Insomnia Thus, another aspect of the invention, to the use of compound of Formula I for the preparation of a medicament for sleep disorders such as insomnia.

In light of the distribution of the receptor in cardiovascular tissue, the use of compound of Formula I for the preparation of a medicament acting through the activation of urotensin receptor II signaling for regulating blood pressure in a mammal is a particularly interesting aspect of the invention as well as the use of compound of Formula I for the preparation of a medicament acting through the activation of urotensin receptor II signaling for altering the heart rate or cardiac output in a mammal. Correspondingly, a method of altering the vascular pressure in a mammal, comprising constricting or dilating vascular tissue in said mammal, the constricting or dilating is performed by the activation of urotensin receptor signaling, said activation being performed by the administration of an effective amount compound of Formula I is anticipated. Moreover, method of altering the heart rate in a mammal, comprising the activation of a urotensin receptor, said activating being performed by the administration of an effective amount compound of Formula I is also anticipated.

Moreover, the use of compound of Formula I for the preparation of a diuretic agent acting through the activation of urotensin receptor II signaling is also anticipated.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

Another embodiment is a method of identifying a compound which regulates activity of an Urotensin II receptor by culturing cells that express the Urotensin II receptors; incubating the cells with at least one compound of Formula I as defined herein; and determining any change in activity of the Urotensin II receptor so as to identify a compound of Formula I which regulates activity of a Urotensin II receptor.

Another embodiment is a pharmaceutical composition comprising a compound of Formula I as described above, and a physiologically acceptable carrier, diluent, or excipient, or a combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990, which is hereby incorporated by reference in its entirety.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternately, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., as disclosed in Remington's Pharmaceutical Sciences, cited above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g. dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increases the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; and other biocompatible polymers may replace polyethylene glycol, e.g., polyvinyl pyrrolidone. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. In some embodiments, the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXAMPLES

Embodiments of the present invention are disclosed in further detail in the following examples, which are not in any way intended to limit the scope of the invention.

### Example 1

### General Analytical LC-MS Procedure

Procedure 1 (AP1): The analysis was performed on a combined prep/analytical Waters/Micromass system consisting of a ZMD single quadrupole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammonium acetate in water, buffer B: 10 mM ammonium acetate in acetonitrile/water 95/5. A gradient was run from 30% B to 100% B in 10 min, dwelling at 100% B for 1 min, and re-equilibrating for 6 min. The system was operated at 1 ml/min.

Procedure 2 (AP2): The analysis was performed on a combined prep/analytical Waters/Micromass system consisting of a ZMD single quadrupole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammonium acetate in water, buffer B: 10 mM ammonium acetate in acetonitrile/water 95/5. A gradient was run from 30% B to 100% B in 7 min, dwelling at 100% B for 1 min, and re-equilibrating for 5.5 min. The system was operated at 1 ml/min.

### Example 2

### General Gas Chromatography (GC) Procedure

GC method 50 was used. Method 50 starts at 50° C. and has a gradient of 20° C./min until 250° C. then holds the temperature for 5 minutes. The analysis was performed on an Aglient 6850 series GC system with capillary S/SL inlet and FID with EPC installation. The column was a 30 m×0.32 mm×0.25 μm HP5 column.

### Example 3

### Synthesis of the Compounds of the Invention

General: All chemicals were purchased from Aldrich, Acros or Maybridge and were used without purification. ¹H (400 MHz) and ¹³C (100 MHz) NMR spectra were recorded in CDCl₃ unless otherwise stated using a JEOL JMN-ECP400 instrument. All reactions were followed by TLC (Merck silica gel 60 F₂₅₄) and analyzed under UV (254 nm). In case of flash chromatography, Merck silica gel 60 (230-400 mesh) was used. Melting points were recorded on a Büchi melting point B-545 apparatus and are uncorrected. Elemental analyses were performed at Kolbe Analytishe Laboratorium, Mülheim an der Ruhr, Germany. FAB MS spectra were obtained from Stenhagen Analyslab AB, Mölndal, Sweden, using a VG 7070E magnetic sector instrument (VG Analytical/Micromass, Manchester UK). Conditions for FAB (fast atom bombardment): Xe gun at 8 kV, matrix glycerol or 3-nitrobenzylalcohol with PEG 600 as mass reference. A signal from a coil in the magnet field was used for mass calibration. Acceleration voltage 5 kV. Magnet scan from 150 to 700 in 4 s (typical).

### 1-(4-Chlorophenyl)-3-dimethylamino-propan-1-ol (1a)

1-(4-Chlorophenyl)-3-dimethylamino-propan-1-one (3.0 g, 14.2 mmol) was dissolved in THF (250 mL) and LiAlH₄ (0.68 g, 18 mmol) was added. The solution was stirred at room temperature for 2 h. A saturated aqueous NaHCO₃ solution was slowly added and the mixture was extracted twice with EtOAc. The combined organic phases were washed (water, brine) and concentrated to afford the title compound (2.75 g, 91%) as a pale yellow oil which solidified slowly upon standing. ¹H NMR δ 1.69-1.83 (m, 2H), 2.29 (s, 6H), 2.42-2.48 (m, 1H), 2.60-2.66 (m, 1H), 4.91 (dd, 1H, J=4.8, 8.4 Hz), 7.30 (app s, 4H). ¹³C NMR δ 34.5, 45.4 (2 C:s), 58.5, 75.3, 127.0 (2 C:s), 128.4 (2 C:s), 132.5, 143.7.

### 1-(4-Chlorophenyl)-3-dimethylamino-propan-1-amine (2a)

A solution of alcohol 1a (3.0 g, 14 mmol) in CH₃CN (6 mL) was cooled to −15° C. Conc H₂SO₄ (15 mL) was added and the solution was stirred for 18 h. Water (45 mL) was slowly added to the reaction and the mixture was basified to pH 14 using NaOH pellets and extracted twice with EtOAc. The combined organic phases were washed (water, brine) and concentrated. The resulting yellowish oil was refluxed for 18 h in 6M HCl (50 mL). The reaction mixture was again basified to pH 14 using NaOH pellets and extracted twice with EtOAc. The combined organic phases were washed (water, brine) and concentrated to yield 3.10 g (quant) of the title compound as a yellow oil which was used without further purification. ¹H NMR δ 1.72-1.78 (m, 2H), 2.29 (s, 6H), 2.40-2.45 (m, 1H), 2.61-2.67 (m, 1H), 4.90 (dd, 1H, J=6.0, 11.4 Hz), 7.31 (app s, 4H). ¹³C NMR δ 37.3, 45.6 (2 C:s), 54.3, 57.0, 127.8 (2 C:s), 128.7 (2 C:s), 132.5, 145.1.

### [3-(4-Chlorophenyl)-3-(4-methylbenzyloxy)-propyl]-N,N-dimethyl amine (3a)

Alcohol 1a (0.15 g, 0.7 mmol) was dissolved in CH₂Cl₂. PS-DIPEA (1 g, 3 mmol amine/g) and 4-methyl-benzyl bromide (0.13 g, 0.7 mmol) were added and the solution was shaken for 48 h. The solution was filtered, concentrated and purified using flash chromatography (first CH₂Cl₂ 100%, thereafter a gradient up to 50% MeOH). The fractions containing product were pooled and concentrated. The residue was dissolved in diethyl ether and HCl₍ₑₜₕₑᵣ₎ was added. Evaporation of the solvent afforded the title product as a white hygroscopic solid (120 mg, 48%). ¹H NMR δ 2.26-2.33 (m, 2H), 2.36 (s, 3H), 3.08 (s, 3H), 3.10 (s, 3H), 3.80-3.99 (m, 2H), 4.67 (bs, 2H), 4.95 (dd, 1H, J=4.3, 7.5 Hz), 7.17 (d, 2H, J=6.4 Hz), 7.20-7.30 (m, 2H), 7.33-7.40 (m, 4H). ¹³C NMR δ 18.9, 32.6, 49.6, 49.9, 62.4, 67.8, 69.7, 123.8, 127.4 (2 C:s), 128.6 (2 C:s), 130.0 (2 C:s), 133.0 (2 C:s), 133.2, 141.3, 142.3. HRFABMS 317.164 (C₁₉H₂₄ClNO requires 317.155)

### [3-(4-Chlorophenyl)-3-(2-methoxybenzyloxy)-propyl]-N,N-dimethyl amine (3b)

Alcohol 1a (0.2 g, 1 mmol) was dissolved in CH₂Cl₂. PS-DIPEA (1 g, 3 mmol amine/g) and 2-methoxy-benzyl chloride (0.15 g, 1 mmol) were added and the solution was shaken for 48 h. The solution was filtered, concentrated and purified using flash chromatography (first CH₂Cl₂ 100%, thereafter a gradient up to 50% MeOH). The fractions containing product were pooled and concentrated. The residue was dissolved in diethyl ether and HCl₍ₑₜₕₑᵣ₎ was added. Evaporation of the solvent afforded the title product as a white hygroscopic solid (290 mg, 78%). ¹H NMR δ 2.29-2.34 (m, 2H), 3.05 (s, 6H), 3.78 (s, 3H), 3.82-3.85 (m, 2H), 4.55 (s, 2H), 4.94 (dd, 1H, J=4.1, 7.7 Hz), 6.90 (d, 1H, J=8.4 Hz), 6.95 (dd, 1H, J=7.3, 8.1 Hz), 7.19 (d, 2H, J=8.4 Hz), 7.39-7.42 (m, 3H), 7.49 (d, 1H, J=7.3 Hz). ¹³C NMR δ 32.6, 50.0, 50.2, 55.8, 62.4, 63.2, 69.6, 111.3, 115.5, 121.3, 127.5 (2 C:s), 128.5 (2 C:s), 132.8, 132.9, 135.5, 142.8, 158.7. Anal calc for C₁₉H₂₅ClN₂O×1.5H₂O, C, 57.4; N, 3.5; H, 7.0, found: C, 57.6; N, 3.3; H, 7.0.

### 3-Dimethylamino-1-(4-methylphenyl)-propanol (1b)

3-(dimethylamino)-1-p-tolylpropan-1-one (3.6 g, 18.8 mmol) was dissolved in THF (250 ml). LAH (0.72 g, 18.8 mmol) was added slowly and the mixture was stirred for 18 h. NaOH (1 M) (100 ml) was added dropwise until pH 14. The resulting mixture was extracted with EtOAc (150 ml+100 ml). The organic phases were combined, washed with water (200 ml) and brine (200 ml) and concentrated to yield the title product as a yellow oil (3.3 g, 91%). ¹H NMR (CDCl₃) δ 1.78-1.82 (m, 2H), 2.30 (s, 3H), 2.35 (s, 6H), 2.44-2.50 (m, 1H), 2.62-2.69 (m, 1H), 4.90 (dd, 1H, J=7.2, 12.0 Hz), 7.15 (d, 2H, J=7.6 Hz), 7.27 (d, 2H, J=7.6 Hz). ¹³C NMR (CDCl₃) δ 21.4, 34.8, 45.6 (2 C:s), 58.7, 75.9, 125.7 (2 C:s), 129.1 (2 C:s), 136.6, 142.4.

### 3-Dimethylamino-1-(4-methylphenyl)propanamine (2b)

Compound 1b (3.3 g, 17.1 mmol) was dissolved in acetonitrile (6 ml) and stirred on an ice-salt bath. H₂SO₄ (15 ml) was added slowly. After 18 h NaOH pellets were added until pH 14. The mixture was extracted with EtOAc (2×150 ml). The organic phases were combined and washed with water (200 ml) and brine (200 ml) and concentrated to obtain the corresponding acetamide as a yellow oil. HCl (6 M) (50 ml) was then added to the intermediate and the solution was refluxed for 3 days. H₂O (100 ml) and NaOH pellets were added slowly to the mixture until pH 14. The mixture was extracted with EtOAc (2×100 ml) and the organic phases were combined and washed with water (100 ml) and brine (100 ml) and concentrated to yield the title product as a yellow oil (1.55 g, 47%). ¹H NMR (CDCl₃) δ 1.81-1.87 (m, 2H), 2.23 (s, 6H), 2.28-2.36 (m, 2H), 2.32 (s, 3H), 3.96 (dd, 1H, J=6.8, 7.2 Hz), 7.14 (d, 2H, J=8.4 Hz), 7.22 (d, 2H, J=8.4 Hz). ¹³C NMR (CDCl₃) δ 21.3, 36.9, 45.7 (2 C:s), 54.8, 57.4, 126.4 (2 C:s), 129.4 (2 C:s), 136.8, 143.4.

### 3-Dimethylamino-1-(2-naphthyl)propanol (1c)

3-(dimethylamino)-1-(naphthalen-2-yl)propan-1-one (4.0 g, 17.6 mmol) was dissolved in THF (250 ml). LAH (0.67 g, 17.6 mmol) was added slowly and the mixture was stirred for 18 h. NaOH (1 M) (100 ml) was then added dropwise until pH 14. The resulting mixture was extracted with EtOAc (150 ml+100 ml). The organic phases were combined, washed with water (200 ml) and brine (100 ml) and concentrated to yield the title product as a yellow oil (4.1 g, quant). ¹H NMR (CDCl₃) δ 1.86-2.02 (m, 2H), 2.32-2.42 (m, 1H) 2.38 (s, 6H), 2.54-2.63 (m, 1H), 5.11 (dd, 1H, J=4.0, 6.1 Hz), 7.40-7.52 (m, 3H), 7.76-7.90 (m, 4H); ¹³C NMR (CDCl₃) δ 34.5, 45.4 (2 C:s), 58.4, 75.8, 124.1, 124.3, 125.6, 126.0, 127.7, 128.0, 128.1, 132.8, 133.5, 142.6.

### 3-Dimethylamino-1-(2-naphthyl)propanamine (2c)

Compound 1c (4.1 g, 17.6 mmol) was dissolved in acetonitrile (6 ml) and stirred on an ice-salt bath. H2SO4 (15 ml) was added slowly. After 18 h NaOH pellets were added until pH 14. The mixture was extracted with EtOAc (2×150 ml). The organic phases were combined and washed with water (200 ml) and brine (200 ml) and concentrated to obtain the corresponding acetamide as yellow oil. HCl (6 M) (50 ml) was then added to the intermediate and the solution was refluxed for 3 days. H2O (100 ml) and NaOH pellets were added slowly to the mixture until pH 14. The mixture was extracted with EtOAc (2×100 ml) and the organic phases were combined and washed with water (100 ml) and brine (100 ml) and concentrated to yield the title product as a yellow oil (3.05 g, 76%). ¹H NMR (CDCl3) δ 1.88-1.95 (m, 2H), 2.15 (s, 6H), 2.24-2.39 (m, 2H), 4.15 (dd, 1H, J=6.6, 13.6 Hz), 7.41-7.50 (m, 3H), 7.74-7.77 (m, 1H), 7.79-7.86 (m, 3H). ¹³C NMR (CDCl3) δ 37.1, 45.7 (2 C:s), 55.1, 57.2, 124.8 (2 C:s), 126.1 (2 C:s), 127.7, 127.9, 128.3, 132.8, 133.4, 143.9.

General Procedure for the Synthesis of Ester Derivatives 4a-4q

EDC (96 mg, 0.5 mmol), DMAP (12 mg, 0.01 mmol) and the appropriate carboxylic acid (0.47 mmol) were added to a solution of alcohol 1a (0.1 g, 0.47 mmol) in CH₂Cl₂ (15 mL), and the solution was stirred at room temperature over night. 1M NaOH (15 mL) was added to the mixture, which was stirred for 15 min and then extracted twice with EtOAc. The combined organic phases were washed (water and brine) and concentrated. The crude product was purified using flash chromatography (first CH₂Cl₂ 100%, thereafter a gradient up to 50% MeOH). The pure products 4a-o were converted to the corresponding hydrochloride salts, and 4p-q to their oxalic salt, for analysis, storage and biological testing.

### 1-(4-Chlorophenyl)-3-dimethylamino-propyl 2-methyl-benzoate HCl (4a)

Reaction of 2-methyl-benzoic acid with 1a yielded 90 mg free amine (58%) which was converted to the hydrochloride salt. ¹H NMR δ 2.51 (s, 3H), 2.53-2.69 (m, 2H), 2.79 (s, 3H), 2.80 (s, 3H), 3.01-3.11 (m, 2H), 5.99 (dd, 1H, J=7.0, 13.2 Hz), 7.24-7.45 (m, 7H), 7.95 (d, 1H, J=8.0 Hz), 12.95 (bs, 1H). ¹³C NMR δ 21.9, 30.9, 43.0, 43.3, 54.8, 72.5, 126.0, 127.8 (2 C:s), 128.3, 129.3 (2 C:s), 130.7, 132.1, 132.8, 134.8, 137.0, 141.1, 166.1. HRFABMS 332.147 (C₁₉H₂₂ClNO₂ requires 332.142)

### 1-(4-Chlorophenyl)-3-dimethylamino-propyl 2-ethyl-benzoate HCl (4b)

Reaction of 2-ethyl-benzoic acid with 1a yielded 100 mg free amine (62%) which was converted to the hydrochloride salt. ¹H NMR δ 1.18 (t, 3H, J=7.5 Hz), 1.95-2.05 (m, 1H), 2.18 (s, 6H), 2.29-2.38 (m, 3H), 2.92 (q, 2H, J=7.5 Hz), 5.99 (dd, 1H, J=6.2, 13.3 Hz), 7.22-7.27 (m, 2H), 7.31-7.37 (m, 4H), 7.40-7.45 (m, 1H), 7.87 (d, 1H, J=8.1 Hz), 12.80 (bs, 1H). ¹³C NMR δ 15.9, 27.4, 34.6, 45.5 (2 C:s), 55.7, 74.4, 125.8, 128.0 (2 C:s), 128.8 (2 C:s), 130.3, 130.4, 131.6, 132.2, 133.7, 139.0, 146.1, 166.9. HRFABMS 346.165 (C₂₀H₂₄ClNO₂ requires 346.157). Anal calc for C₂₀H₂₄ClNO₂, C, 62.8; H, 6.6; N, 3.7. Found: C, 62.7; H, 6.9; N, 3.9.

### 1-(4-Chlorophenyl)-3-dimethylamino-propyl 4-methyl-benzoate HCl (4c)

Reaction of 4-methyl-benzoic acid with 1a yielded 90 mg free amine (58%) which was converted to the hydrochloride salt. ¹H NMR δ 2.41 (s, 3H), 2.49-2.69 (m, 2H), 2.79 (s, 3H), 2.80 (s, 3H), 3.03-3.12 (m, 2H), 6.03 (dd, 1H, J=3.0, 8.0 Hz), 7.24-7.42 (m, 6H), 7.91 (d, 2H, J=8.4 Hz), 12.90 (bs, 1H). ¹³C NMR δ 21.7, 31.1, 43.1, 43.3, 54.8, 72.7, 126.4, 127.8 (2 C:s), 129.2 (2 C:s), 129.4 (2 C:s), 129.8 (2 C:s), 134.7, 137.0, 144.7, 165.6. HRFABMS 332.145 (C₁₉H₂₂ClNO₂ requires 332.142)

### 1-(4-Chlorophenyl)-3-dimethylamino-propyl 2,3-dimethyl-benzoate HCl (4d)

Reaction of 2,3-dimethyl-benzoic acid with 1a yielded 140 mg free amine (86%) which was converted to the hydrochloride salt. ¹H NMR δ 2.32 (s, 6H), 2.39 (s, 3H), 2.50-2.65 (m, 2H), 2.81 (s, 3H), 3.04-3.12 (m, 2H), 6.00 (dd, 1H, J=5.8, 11.2 Hz), 7.14 (d, 1H, J=7.6 Hz), 7.29-7.48 (m, 5H), 7.68 (d, 1H, J=8.0 Hz), 12.70 (bs, 1H). ¹³C NMR δ 16.9, 20.7, 31.3, 43.9, 44.1, 55.1, 72.8, 125.5, 126.4, 128.0 (2 C:s), 129.1, 129.3 (2 C:s), 129.7, 133.9, 134.7, 137.1, 138.4, 167.1. HRFABMS 346.151 (C₂₀H₂₄ClNO₂ requires 346.157).

### 1-(4-Chlorophenyl)-3-dimethylamino-propyl 3-methoxy-2-methyl-benzoate HCl (4e)

Reaction of 3-methoxy-2-methyl-benzoic acid with 1a yielded 120 mg free amine (71%) which was converted to the hydrochloride salt. ¹H NMR δ 2.36 (s, 3H), 2.50-2.62 (m, 2H), 2.79 (s, 6H), 3.02-3.12 (m, 2H), 3.82 (s, 3H), 6.00 (dd, 1H, J=6.2, 11.4 Hz), 6.99 (d, 1H, J=8.0 Hz), 7.20 (dd, 1H, J=7.7 Hz, 8.1 Hz), 7.32-7.43 (m, 5H), 12.75 (bs, 1H). ¹³C NMR δ 12.8, 31.1, 43.1, 43.4, 54.7, 55.9, 72.8, 114.1, 122.0, 126.4, 127.9 (2 C:s), 129.2, 129.3 (2 C:s), 130.4, 134.7, 137.1, 158.2, 166.6. HRFABMS 362.156 (C₂₀H₂₄ClNO₃ requires 362.152)

### 1-(4-Chlorophenyl)-3-dimethylamino-propyl 3-chloro-2-methyl-benzoate HCl (4f)

Reaction of 3-chloro-2-methyl-benzoic acid with 1a yielded 80.0 mg free amine (46%) which was converted to the hydrochloride salt. ¹H NMR δ 2.45-2.62 (m, 5H), 2.79 (s, 6H), 3.00-3.10 (m, 2H), 5.99 (dd, 1H, J=5.6, 13.2 Hz), 7.19 (dd, 1H, J=7.5, 8.0 Hz), 7.35 (d, 2H, J=5.5 Hz), 7.37 (d, 2H, J=5.5 Hz), 7.51 (d, 1H, J=8.0 Hz), 7.72 (d, 1H, J=7.5 Hz), 12.80 (bs, 1H). ¹³C NMR δ 17.6, 31.0, 43.1, 43.3, 54.7, 73.3, 126.7, 128.0 (2 C:s), 128.7, 129.4 (2 C:s), 131.3, 133.3, 134.9, 136.4, 136.7, 137.8, 166.0. HRFABMS 366.098 (C₁₉H₂₁Cl₂NO₂ requires 366.102).

### 1-(4-Chlorophenyl)-3-dimethylamino-propyl 4-bromo-2-methyl-benzoate HCl (4g)

Reaction of 4-bromo-2-methyl-benzoic acid with 1a yielded 100 mg free amine (52%) which was converted to the hydrochloride salt. ¹H NMR δ 2.44-2.68 (m, 2H), 2.53 (s, 3H), 2.72 (s, 6H), 2.97-3.10 (m, 2H), 6.01 (dd, 1H, J=5.2, 11.0 Hz), 7.31-7.46 (m, 6H), 7.82 (d, 1H, J=8.8 Hz), 12.80 (bs, 1H). ¹³C NMR δ 21.8, 31.0, 43.1, 43.4, 54.8, 73.1, 126.4, 127.2, 127.6, 128.0 (2 C:s), 129.3, 129.4 (2 C:s), 132.3, 134.9, 136.8, 143.1, 165.5. Anal calc for C₁₉H₂₂BrCl₂NO₂, C, 50.9; H, 5.0; N, 3.1. Found: C, 51.0; H, 4.9; N, 3.1.

### 1-(4-Chlorophenyl)-3-dimethylamino-propyl 2,5-dimethyl-benzoate HCl (4h)

Reaction of 2,5-dimethyl-benzoic acid with 1a yielded 130 mg free amine (80%) which was converted to the hydrochloride salt. ¹H NMR δ 2.37 (s, 3H), 2.50 (s, 3H), 2.53-2.69 (m, 2H), 2.79 (s, 3H), 2.81 (s, 3H), 3.04-3.09 (m, 2H), 6.00 (dd, 1H, J=6.0, 11.2 Hz), 7.13 (d, 1H, J=7.6 Hz), 7.23-7.26 (m, 1H), 7.35-7.42 (m, 4H), 7.74 (s, 1H), 12.95 (bs, 1H). ¹³C NMR δ 20.8, 21.5, 30.9, 42.9, 43.2, 54.9, 72.6, 127.9 (2 C:s), 128.1, 129.2 (2 C:s), 131.0, 131.9, 133.6, 134.7, 135.6, 137.0, 137.8, 166.2. HRFABMS 346.150 (C₂₀H₂₄ClNO₂ requires 346.157)

### 1-(4-Chlorophenyl)-3-dimethylamino-propyl 2,4,5-trimethyl-benzoate HCl (4i)

Reaction of 2,4,5-trimethyl-benzoic acid with 1a yielded 150 mg free amine (89%) which was converted to the hydrochloride salt. ¹H NMR δ 2.24 (s, 3H), 2.26 (s, 3H), 2.46 (s, 3H), 2.51-2.62 (m, 2H), 2.76 (s, 3H), 2.80 (s, 3H), 3.01-3.10 (m, 2H), 5.98 (dd, 1H, J=2.2, 8.0 Hz), 6.99 (s, 1H), 7.31 (d, 2H, J=8.4 Hz), 7.36 (d, 2H, J=8.4 Hz), 7.70 (s, 1H), 12.70 (bs, 1H). ¹³C NMR δ 19.3, 19.8, 21.6, 31.1, 43.1, 43.3, 54.8, 72.4, 125.5, 127.9 (2 C:s), 129.2 (2 C:s), 131.8, 133.5, 134.3, 134.6, 137.3, 138.4, 142.3, 166.3. Anal calc for C₂₁H₂₇Cl₂NO₂, C, 63.6; H, 6.9; N, 3.5. Found: C, 63.5; H, 7.0; N, 3.6.

### 1-(4-Chlorophenyl)-3-dimethylamino-propyl 3-methyl-thiophene-2-carboxylate HCl (4j)

Reaction of 3-methyl-thiophene-2-carboxylic acid with 1a yielded 150 mg free amine (95%) which was converted to the hydrochloride salt. ¹H NMR δ 2.50 (s, 3H), 2.53-2.58 (m, 2H), 2.80 (s, 6H), 3.06-3.11 (m, 2H), 5.99 (dd, 1H, J=5.6, 7.2 Hz), 6.93 (d, 1H, J=5.2 Hz), 7.33-7.44 (m, 5H), 12.80 (bs, 1H). ¹³C NMR δ 16.1, 31.1, 42.9, 43.4, 54.8, 72.6, 125.3, 127.7 (2 C:s), 129.3 (2 C:s), 131.0, 132.2, 134.6, 136.9, 147.8, 161.4. HRFABMS 338.102 (C₁₇H₂₀ClNO₂S requires 338.098)

### 1-(4-Chlorophenyl)-3-dimethylamino-propyl naphthalene-1-carboxylate HCl (4k)

Reaction of naphthalene-1-carboxylic acid with 1a yielded 60 mg free amine (35%) which was converted to the hydrochloride salt. ¹H NMR δ 2.57-2.85 (m, 2H), 2.84 (s, 6H), 3.09-3.13 (m, 2H), 6.14 (dd, 1H, J=6.1, 13.2 Hz), 7.36 (d, 2H, J=8.1 Hz), 7.43 (d, 2H, J=8.1 Hz), 7.51-7.56 (m, 2H), 7.58-7.62 (m, 1H), 7.88 (d, 1H, J=7.7 Hz), 8.06 (d, 1H, J=8.4 Hz), 8.25 (d, 1H, J=7.0 Hz), 8.85 (d, 1H, J=8.4 Hz), 12.95 (bs, 1H). ¹³C NMR δ 31.1, 43.0, 43.3, 54.8, 72.9, 124.6, 125.5, 125.7, 126.6, 127.9 (2 C:s), 128.3, 128.8, 129.4 (2 C:s), 130.7, 131.6, 133.9, 134.4, 134.9, 137.0, 166.1. Anal calc for C₂₂H₂₃Cl₂NO₂, C, 65.4; H, 5.7; N, 3.5. Found: C, 65.3; H, 5.6; N, 3.6.

### 1-(4-Chlorophenyl)-3-dimethylamino-propyl naphthalene-2-carboxylate HCl (4l)

Reaction of naphthalene-2-carboxylic acid with 1a yielded 170 mg free amine (98%) which was converted to the hydrochloride salt. ¹H NMR δ 2.02-2.11 (m, 1H), 2.22-2.45 (m, 9H), 6.07 (dd, 1H, J=6.2, 13.5 Hz), 7.33 (d, 2H, J=8.4 Hz), 7.41 (d, 2H, J=8.4 Hz), 7.52-7.61 (m, 2H), 7.84-7.90 (m, 2H), 7.96 (d, 1H, J=7.3 Hz), 8.05 (dd, 1H, J=2.6, 8.0 Hz), 8.61 (s, 1H), 12.70 (bs, 1H). ¹³C NMR δ 34.3, 45.6 (2 C:s), 55.7, 74.6, 125.2, 126.8, 127.4, 127.8, 128.0 (2 C:s), 128.3, 128.5, 128.9 (2 C:s), 129.4, 131.2, 132.5, 133.9, 135.7, 139.1, 165.9. HRFABMS 368.138 (C₂₂H₂₂ClNO₂ requires 368.142)

### 1-(4-Chlorophenyl)-3-dimethylamino-propyl 4-phenyl-benzoate HCl (4m)

Reaction of 4-phenyl-benzoic acid with 1a yielded 80 mg free amine (43%) which was converted to the hydrochloride salt. ¹H NMR δ 2.56-2.66 (m, 2H), 2.82 (s, 6H), 3.12-3.28 (m, 2H), 6.05 (dd, 1H, J=6.0, 13.0 Hz), 7.33-7.47 (m, 7H), 7.59 (d, 2H, J=7.7 Hz), 7.66 (d, 2H, J=7.3 Hz), 8.09 (d, 2H, J=7.7 Hz), 12.50 (bs, 1H). ¹³C NMR δ 31.2, 43.4, 43.5, 54.9, 72.9, 127.4 (2 C:s), 127.8 (2 C:s), 127.9, 128.4 (2 C:s), 129.0, 129.1 (2 C:s), 129.3 (2 C:s), 130.4 (2 C:s), 134.7, 137.1, 139.8, 146.5, 165.5. HRFABMS 394.156 (C₂₄H₂₄ClNO₂ requires 394.157).

### 1-(4-Chlorophenyl)-3-dimethylamino-propyl 1-methyl-indole-2-carboxylate HCl (4n)

Reaction of 1-methyl-indole-2-carboxylic acid with 1a yielded 120 mg of the free amine (69%) which was converted to the hydrochloride salt. ¹H NMR δ 2.54-2.65 (m, 2H), 2.80 (s, 6H), 3.09-3.12 (m, 2H), 4.00 (s, 3H), 6.02 (dd, 1H, J=7.0, 8.0 Hz), 7.13-7.17 (m, 1H), 7.32-7.40 (m, 7H), 7.67 (d, 1H, J=8.0 Hz), 12.80 (bs, 1H). ¹³C NMR δ 31.2, 31.7, 43.1, 43.4, 54.8, 72.4, 110.3, 110.9, 120.9, 122.6, 125.7, 126.1, 126.6, 127.6 (2 C:s), 129.3 (2 C:s), 134.6, 137.3, 140.1, 160.9. HRFABMS 371.147 (C₂₁H₂₃ClN₂O₂ requires 371.152)

### 1-(4-Chlorophenyl)-3-dimethylamino-propyl 1-methyl-indole-3-carboxylate HCl (4o)

Reaction of 1-methyl-indole-3-carboxylic acid with 1a yielded 110 mg free amine (63%) which was converted to the hydrochloride salt. ¹H NMR δ 2.50-2.67 (m, 2H), 2.79 (s, 6H), 3.14-3.20 (m, 2H), 3.85 (s, 3H), 6.03 (dd, 1H, J=5.8, 12.4 Hz), 7.25-7.41 (m, 7H), 7.93 (s, 1H), 8.09 (d, 1H, J=8.1 Hz), 12.65 (bs, 1H). ¹³C NMR δ 31.5, 33.8, 43.4 (2 C:s), 55.1, 71.2, 105.7, 110.2, 121.3, 122.4, 123.2, 126.6, 127.8 (2 C:s), 129.2 (2 C:s), 134.4, 136.3, 137.4, 137.8, 163.7. HRFABMS 371.154 (C₂₁H₂₃ClN₂O₂ requires 371.153).

### 1-(4-Chlorophenyl)-3-dimethylamino-propyl 4-methoxy-benzoate oxalate (4p)

Reaction of 4-methoxy-benzoic acid with 1a yielded 75 mg free amine (46%) which was converted to the oxalate salt as a yellow oil. ¹H NMR (CD₃OD) δ 2.29-2.37 (m, 1H), 2.45-2.52 (m, 1H), 2.88 (s, 6H), 3.18-3.26 (m, 2H), 3.86 (s, 3H), 6.00 (dd, 1H, J=4.8 Hz, 8.1 Hz), 7.01 (d, 2H, J=8.4 Hz), 7.39 (d, 2H, J=8.1 Hz), 7.46 (d, 2H, J=8.1 Hz), 8.02 (d, 2H, J=8.4 Hz). ¹³C NMR (CD₃OD) δ 31.2, 42.3 (2 C:s), 54.2, 54.7, 72.7, 113.7 (2 C:s), 121.7, 127.7 (2 C:s), 128.6 (2 C:s), 131.5 (2 C:s), 133.9, 138.4, 164.2, 164.8, 165.4.

### 1-(4-Chlorophenyl)-3-dimethylamino-propyl 4-trifluoromethyl-benzoate oxalate (4q)

Reaction of 4-trifluoromethyl-benzoic acid with 1a yielded 60 mg free amine (33%) which was converted to the oxalate salt. Mp 186.5-188.0° C. ¹H NMR (CD₃OD) δ 2.33-2.41 (m, 1H), 2.51-2.61 (m, 1H), 2.88 (s, 6H), 3.18-3.26 (m, 2H), 6.06 (dd, 1H, J=4.8, 8.4 Hz), 7.40 (d, 2H, J=8.4 Hz), 7.48 (d, 2H, J=8.4 Hz), 7.81 (d, 2H, J=8.0 Hz), 8.25 (d, 2H, J=8.0 Hz). ¹³C NMR (CD₃OD) δ 30.9, 42.2 (2 C:s), 54.2, 73.8, 123.8 (q, ¹J_{CF3}=271.4 Hz), 125.4 (q, 2C:s, ¹J_{CF3}=3.1 Hz), 127.8 (2 C:s), 128.6 (2 C:s), 130.1 (2 C:s), 133.2, 134.2, 134.4 (q, ²J_{CF3}=32.2 Hz), 137.9, 164.3, 165.4. Anal calc for C₂₁H₂₁ClF₃NO6 C, 53.0; H, 4.4; N, 2.9. Found: C, 53.1; H, 4.4; N, 2.8.

### N-[1-(4-Chlorophenyl)-3-dimethylamino-propyl]-benzamide HCl (5a)

A solution of alcohol 1a (0.1 g, 0.47 mmol) in benzonitrile (0.05 mL, 0.5 mmol) was cooled to −15° C. Conc H₂SO₄ (15 mL) was added and the solution was stirred for 18 h. Water (45 mL) was slowly added to the reaction and the mixture was basified to pH 14 using NaOH pellets and extracted twice with EtOAc. The combined organic phases were washed (water, brine) and concentrated. Flash chromatography using CH₂Cl₂/MeOH/NEt₃ (89.9/10/0.1) as eluent afforded the pure compound that was converted to the corresponding HCl-salt obtained as a yellow oil (48 mg, 27%). ¹H NMR (CD₃OD) δ 2.31-2.45 (m, 2H), 2.90 (s, 6H), 3.12-3.31 (m, 2H), 4.87 (s, 1H), 5.22 (dd, 1H, J=5.5, 9.5 Hz), 7.37 (d, 2H, J=8.4 Hz), 7.41-7.48 (m, 4H), 7.52-7.57 (m, 1H), 7.86 (d, 2H, J=7.0 Hz). ¹³C NMR (CD₃OD) δ 30.3, 42.3 (2 C:s), 50.9, 55.3, 127.3 (2 C:s), 128.2 (2 C:s), 128.3 (2 C:s), 128.6 (2 C:s), 131.7, 132.3, 133.8, 140.1, 168.8. Anal calc for C₁₈H₂₁ClN₂O, C, 61.2; H, 6.3; N, 7.9. Found: C, 60.8; H, 6.4; N, 7.5.

General Procedure for the Synthesis of Amide Derivatives 5b-5q

The benzoic acid was dissolved in THF (75 mL/g) and triethylamine (2 eqv) was added. Under vigorous stirring SOCl₂ (1.1 eqv) was added dropwise and the mixture was stirred at rt for 20 min. A solution of 2 (0.6 eqv) in THF was added slowly and the reaction mixture was stirred for another 2 h. The mixture was poured into NaOH (1M) and extracted twice with EtOAc. The combined organic phases were washed (water, brine) and concentrated. The crude oil was dissolved in CH₂Cl₂ and applied to a SAX-2 ion exchange column, washed with CH₂Cl₂ and MeOH. The product was eluted using methanolic NH₃ (2M), and concentrated. The pure products were converted to the corresponding oxalate salts for analysis, storage and biological testing.

### N-[1-(4-Chlorophenyl)-3-dimethylamino-propyl]-2-methyl-benzamide oxalate (5b)

Reaction of 2-methyl-benzoic acid (210 mg, 1.56 mmol) with 2a yielded 120 mg pure product (35%) which was converted to the oxalate salt obtained as a yellow oil. ¹H NMR (CD₃OD) δ 2.23-2.39 (m, 2H), 2.31 (s, 3H), 2.90 (s, 6H), 3.15-3.28 (m, 2H), 5.17 (dd, 1H, J=5.8, 9.1 Hz), 7.21-7.24 (m, 2H), 7.31-7.38 (m, 2H), 7.39 (d, 2H, J=8.4 Hz), 7.46 (d, 2H, J=8.4 Hz). ¹³C NMR (CD₃OD) δ 18.4, 30.3, 42.2, 42.5, 50.8, 55.3, 125.5, 126.8, 128.2, 128.7 (2 C:s), 129.8, 130.4 (2 C:s), 133.3, 135.5, 136.1, 140.0, 171.4. Anal calc for C₂₁H₂₅ClN₂O₅, C, 59.9; H, 6.0; N, 6.7. Found: C, 60.3; H, 5.9; N, 6.4.

### N-[1-(4-Chlorophenyl)-3-dimethylamino-propyl]-2-ethyl-benzamide oxalate (5c)

Reaction of 2-ethyl-benzoic acid (230 mg, 1.56 mmol) with 2a yielded 135 mg pure product (44%) which was converted to the oxalate salt. Mp 96.4-97.2° C. ¹H NMR (CD₃OD) δ 1.10 (t, 3H, J=7.5 Hz), 2.22-2.35 (m, 2H), 2.65 (dq, 2H, J=2.5, 7.5 Hz), 2.88 (s, 6H), 3.12-3.27 (m, 2H), 5.15 (dd, 1H, J=2.6, 8.8 Hz), 7.21-7.28 (m, 2H), 7.32-7.37 (m, 2H), 7.39 (d, 2H, J=8.8 Hz), 7.43 (d, 2H, J=8.8 Hz). ¹³C NMR (CD₃OD) δ 14.9, 25.9, 30.2, 42.3 (2 C:s), 50.9, 55.3, 125.5, 126.8, 128.2 (2 C:s), 128.6 (2 C:s), 129.0, 129.8, 133.3, 135.8, 139.9, 141.8, 165.3, 171.6. Anal calc for C₂₂H₂₇ClN₂O₅, C, 60.8; H, 6.3; N, 6.4. Found: C, 60.8; H, 6.2; N, 6.3.

### N-[1-(4-Chlorophenyl)-3-dimethylaminopropyl]-4-methoxy-benzamide oxalate (5d)

Reaction of 4-methoxy-benzoic acid (71 mg, 0.47 mmol) with 2a yielded 150 mg pure product (92%), which was converted to the oxalate salt. Mp 172.6-172.9° C. ¹H NMR (CD₃OD) δ 2.27-2.41 (m, 2H), 2.88 (s, 6H), 3.12-3.29 (m, 2H), 3.84 (s, 3H), 5.18 (dd, 1H, J=5.8, 9.2 Hz), 6.98 (d, 2H, J=8.4 Hz), 7.37 (d, 2H, J=8.4 Hz), 7.43 (d, 2H, J=8.4 Hz), 7.83 (d, 2H, J=8.4 Hz). ¹³C NMR (CD₃OD) δ 30.3, 42.3 (2 C:s), 50.9, 54.6, 55.3, 113.4 (2 C:s), 125.8, 128.1 (2 C:s), 128.5 (2 C:s), 129.2 (2 C:s), 133.1, 140.4, 162.8, 163.9, 168.3. Anal. Calcd for C₂₁H₂₅ClN₂O₆: C, 57.7; H, 5.8; N, 6.4. Found: C, 57.6; H, 5.7; N, 6.5.

### N-[1-(4-Chlorophenyl)-3-dimethylaminopropyl]-4-dimethylamino-benzamide oxalate (5e)

Reaction of 4-dimethylamino-benzoic acid (77 mg, 0.47 mmol) with 2a yielded 50 mg pure product (30%) which was converted to the oxalate salt obtained as a yellow oil. ¹H NMR (CD₃OD) δ 2.24-2.38 (m, 2H), 2.86 (s, 6H), 3.00 (s, 6H), 3.10-3.26 (m, 2H), 5.17 (dd, 1H, J=5.2, 9.2 Hz), 6.71 (d, 2H, J=8.8 Hz), 7.35 (d, 2H, J=8.4 Hz), 7.42 (d, 2H, J=8.4 Hz), 7.56 (d, 2H, J=8.8 Hz). ¹³C NMR (CD₃OD) δ 30.4, 38.9 (2 C:s), 42.3 (2 C:s), 50.6, 55.4, 110.7 (2 C:s), 119.9, 128.1 (2 C:s), 128.5 (2 C:s), 128.7 (2 C:s), 133.0, 140.6, 153.2, 168.9. Anal. Calcd for C₂₂H₂₈ClN₃O₅: C, 58.7; H, 6.3; N, 9.3. Found: C, 58.7; H, 6.4; N, 9.3.

### N-[1-(4-Chlorophenyl)-3-dimethylamino-propyl]-2,3-dimethyl-benzamide oxalate (5f)

Reaction of 2,3-dimethyl-benzoic acid (210 mg, 1.40 mmol) with 2a yielded 150 mg pure product (51%) which was converted to the oxalate salt. Mp 176.0-176.4° C. ¹H NMR (CD₃OD) δ 2.17 (s, 3H), 2.23-2.31 (m, 2H), 2.28 (s, 3H), 2.89 (s, 6H), 3.13-3.27 (m, 2H), 5.15 (dd, 1H, J=6.2, 8.8 Hz), 7.10-7.16 (m, 2H), 7.21-7.23 (m, 1H), 7.39 (d, 2H, J=8.8 Hz), 7.43 (d, 2H, J=8.8 Hz). ¹³C NMR (CD₃OD) δ 15.0, 18.7, 30.2, 42.3 (2 C:s), 50.8, 55.3, 124.3, 125.3, 128.2 (2 C:s), 128.6 (2 C:s), 130.9, 133.4, 136.8, 137.5, 139.9, 140.0, 165.4, 172.2. Anal calc for C₂₁H₂₄Cl₂N₂O₅, C, 55.4; H, 5.3; N, 6.2. Found: C, 55.3; H, 5.4; N, 6.1.

### N-[1-(4-Chlorophenyl)-3-dimethylamino-propyl]-3-methoxy-2-methyl-benzamide oxalate (5g)

Reaction of 3-methoxy-2-methyl-benzoic acid (230 mg, 1.40 mmol) with 2a yielded 230 mg pure product (75%) which was converted to the oxalate salt. Mp 198.2-198.6° C. ¹H NMR (CD₃OD) δ 2.11 (s, 3H), 2.21-2.33 (m, 2H), 2.88 (s, 6H), 3.12-3.27 (m, 2H), 3.82 (s, 3H), 5.14 (dd, 1H, J=6.2, 8.8 Hz), 6.91 (d, 1H, J=7.4 Hz), 6.99 (d, 1H, J=8.2 Hz), 7.21 (dd, 1H, J=7.4, 8.2 Hz), 7.39 (d, 2H, J=8.8 Hz), 7.42 (d, 2H, J=8.8 Hz). ¹³C NMR (CD₃OD) δ 11.5, 30.3, 42.3 (2 C:s), 50.8, 54.8, 55.3, 111.2, 118.5, 123.7, 126.6, 128.2 (2 C:s), 128.7 (2 C:s), 133.3, 137.6, 140.0, 158.0, 165.3, 171.4. Anal calc for C₂₂H₂₇ClN₂O₆, C, 58.6; H, 6.0; N, 6.2. Found: C, 58.5; H, 6.1; N, 6.2.

### N-[1-(4-Chlorophenyl)-3-dimethylamino-propyl]-3-chloro-2-methyl-benzamide oxalate (5h)

Reaction of 3-chloro-2-methyl-benzoic acid (510 mg, 3.0 mmol) with 2a yielded 500 mg pure product (76%) which was converted to the oxalate salt. Mp 133.8-134.5° C. ¹H NMR (CD₃OD) δ 2.21-2.36 (m, 2H), 2.30 (s, 3H), 2.89 (s, 6H), 3.12-3.28 (m, 2H), 5.14 (dd, 1H, J=6.2, 8.8 Hz), 7.21-7.29 (m, 2H), 7.39-7.47 (m, 5H). ¹³C NMR (CD₃OD) δ 15.7, 30.2, 42.3 (2 C:s), 50.9, 55.2, 125.3, 126.9, 128.1 (2 C:s), 128.7 (2 C:s), 130.3, 133.2, 133.4, 135.1, 138.6, 139.8, 165.4, 170.4. Anal calc for C₂₁H₂₄Cl₂N₂O₅, C, 55.4; H, 5.3; N, 6.2: Found: C, 55.3; H, 5.3; N, 6.0.

### N-[1-(4-Chlorophenyl)-3′-dimethylamino-propyl]-2,4-dimethyl-benzamide oxalate (5i)

Reaction of 2,4-dimethyl-benzoic acid (210 mg, 1.40 mmol) with 2a yielded 180 mg pure product (62%) which was converted to the oxalate salt. Mp 161.8-162.5° C. ¹H NMR (CD₃OD) δ 2.22-2.34 (m, 2H), 2.28 (s, 3H), 2.31 (s, 3H), 2.88 (s, 6H), 3.12-3.27 (m, 2H), 5.14 (dd, 1H, J=6.6, 9.2 Hz), 7.02-7.06 (m, 2H), 7.25 (d, 1H, J=7.3 Hz), 7.38 (d, 2H, J=8.8 Hz), 7.42 (d, 2H, J=8.8 Hz). ¹³C NMR (CD₃OD) δ 18.4, 19.9, 30.3, 42.3 (2 C:s), 50.8, 55.3, 126.0, 126.9, 128.2 (2 C:s), 128.6 (2 C:s), 131.1, 133.1, 133.3, 135.6, 140.0, 140.2, 165.3, 171.6. Anal calc for C₂₂H₂₇ClN₂O₅, C, 60.8; H, 6.3; N, 6.4. Found: C, 60.8; H, 6.2; N, 6.4.

### N-[1-(4-Chlorophenyl)-3-dimethylamino-propyl]-2,5-dimethyl-benzamide oxalate (5j)

Reaction of 2,5-dimethyl-benzoic acid (230 mg, 1.56 mmol) with 2a yielded 162 mg pure product (44%) which was converted to the oxalate salt. Mp 189.8-190.4° C. ¹H NMR (CD₃OD) δ 2.19-2.35 (m, 2H), 2.24 (s, 3H), 2.31 (s, 3H), 2.87 (s, 6H), 3.12-3.27 (m, 2H), 5.14 (dd, 1H, J=5.9, 9.2 Hz), 7.09-7.16 (m, 3H), 7.38 (d, 2H, J=8.6 Hz), 7.43 (d, 2H, J=8.6 Hz). ¹³C NMR (CD₃OD) δ 17.9, 19.5, 30.3, 42.3 (2 C:s), 50.9, 55.2, 127.2, 128.2 (2 C:s), 128.6 (2 C:s), 130.3, 130.4, 132.3, 133.3, 135.2, 135.9, 140.2, 165.3, 171.6. Anal calc for C₂₂H₂₇ClN₂O₅, C, 60.8; H, 6.3; N, 6.4. Found: C, 60.8; H, 6.2; N, 6.3.

### N-[1-(4-Chlorophenyl)-3-dimethylamino-propyl]-6-chloro-2-methyl-benzamide oxalate (5k)

Reaction of 6-chloro-2-methyl-benzoic acid (240 mg, 1.4 mmol) with 2a yielded 130 mg pure product (42%) which was converted to the oxalate salt obtained as a yellow oil. ¹H NMR (CD₃OD) δ 2.20 (s, 3H), 2.24-2.34 (m, 2H), 2.87 (s, 6H), 3.17-3.29 (m, 2H), 5.18 (dd, 1H, J=5.9, 9.2 Hz), 7.16-7.20 (m, 1H), 7.24-7.27 (m, 2H), 7.38 (d, 2H, J=8.4 Hz), 7.44 (d, 2H, J=8.4 Hz). ¹³C NMR (CD₃OD) δ 18.0, 30.3, 42.4 (2 C:s), 50.9, 55.3, 126.5, 128.4 (2 C:s), 128.5, 128.6 (2 C:s), 130.0, 130.2, 133.4, 136.3, 136.9, 139.6, 165.4, 168.3. Anal calc for C₂₁H₂₄Cl₂N₂O₅, C, 55.4; H, 5.3; N, 6.2: Found: C, 55.3; H, 5.2; N, 6.1.

### N-(1-(4-chlorophenyl)-3-(dimethylamino)propyl)benzo [d][1,3]dioxole-5-carboxamide oxalate (5l)

Reaction of benzo[1,3]dioxole-5-carboxylic acid (78 mg, 0.47 mmol) with 2a yielded 150 mg pure product (88%) which was converted to the oxalate salt. Mp 93.3-94.6° C. ¹H NMR (CD₃OD) δ 2.26-2.40 (m, 2H), 2.88 (s, 6H), 3.11-3.28 (m, 2H), 5.16 (dd, 1H, J=5.5, 9.2 Hz), 6.02 (s, 2H), 6.87 (d, 1H, J=8.4 Hz), 7.33-7.42 (m, 6H). ¹³C NMR (CD₃OD) δ 30.3, 42.3 (2 C:s), 50.1, 55.3, 101.9, 107.3, 107.6, 122.3, 127.6, 128.0 (2 C:s), 128.5 (2 C:s), 133.2, 140.2, 148.0, 150.9, 167.9. Anal calc for C₂₁H₂₃ClN₂O₇, C, 55.9; H, 5.1; N, 6.2. Found: C, 55.6; H, 5.1; N, 6.1.

### N-[1-(4-Chlorophenyl)-3-dimethylaminopropyl]-2,4,5-trimethyl-benzamide oxalate (5m)

Reaction of 2,4,5-trimethyl-benzoic acid (160 mg, 1.0 mmol) with 2a yielded 300 mg pure product (98%) which was converted to the oxalate salt. Mp 183.4-184.5° C. ¹H NMR (CD₃OD) δ 2.20 (s, 3H), 2.22 (s, 3H), 2.24 (s, 3H), 2.25-2.34 (m, 2H), 2.88 (s, 6H), 3.12-3.28 (m, 2H), 5.13 (dd, 1H, J=6.2, 9.2 Hz), 6.99 (s, 1H), 7.13 (s, 1H), 7.38 (d, 2H, J=8.8 Hz), 7.43 (d, 2H, J=8.8 Hz). ¹³C NMR (CD₃OD) δ 17.8, 18.0, 18.3, 30.3, 42.3 (2 C:s), 50.8, 55.3, 127.9, 128.1 (2 C:s), 128.6 (2 C:s), 131.7, 132.9, 133.3, 133.7, 138.6, 140.1, 165.3, 171.6. Anal. Calcd for C₂₃H₂₉ClN₂O₅: C, 61.5; H, 6.5; N, 6.2. Found: C, 61.4; H, 6.5; N, 6.3.

### N-[1-(4-Chlorophenyl)-3-dimethylamino-propyl]-2-naphthyl-carboxamide oxalate (5n)

Reaction of 2-naphthylene carboxylic acid (270 mg, 1.56 mmol) with 2a yielded 170 mg pure product (47%) which was converted to the oxalate salt. Mp 206.0-206.9° C. ¹H NMR (CD₃OD) δ 2.31-2.44 (m, 2H), 2.90 (s, 6H), 3.17-3.28 (m, 2H), 5.26 (dd, 1H, J=5.1, 9.5 Hz), 7.38 (d, 2H, J=8.4 Hz), 7.48 (d, 2H, J=8.4 Hz), 7.53-7.60 (m, 2H), 7.89-8.03 (m, 4H), 8.43 (s, 1H). ¹³C NMR (CD₃OD) δ 30.3, 42.3 (2 C:s), 51.0, 55.3, 123.6, 126.5, 127.4, 127.6, 127.7, 128.0, 128.1 (2 C:s), 128.6 (2 C:s), 128.7, 131.1, 132.7, 133.2, 135.1, 140.2, 163.5, 168.8. Anal calc for C₂₄H₂₅ClN₂O₅, C, 63.1; H, 5.5; N, 6.1. Found: C, 62.9; H, 5.6; N, 6.0.

### N-[1-(4-Chlorophenyl)-3-dimethylamino-propyl]-4-phenyl-benzamide oxalate (5o)

Reaction of 4-phenyl-benzoic acid (310 mg, 1.56 mmol) with 2a yielded 152 mg pure product (50%) which was converted to the oxalate salt. Mp 203.9-205.7° C. ¹H NMR (CD₃OD) δ 2.28-2.45 (m, 2H), 2.88 (s, 6H), 3.14-3.28 (m, 2H), 5.22 (dd, 1H, J=5.5, 9.5 Hz), 7.34-7.38 (m, 3H), 7.43-7.47 (m, 4H), 7.65 (d, 2H, J=7.0 Hz), 7.71, (d, 2H, J=8.4 Hz), 7.94 (d, 2H, J=8.4 Hz). ¹³C NMR (CD₃OD) δ 30.3, 42.3 (2 C:s), 51.0, 55.3, 126.7 (2 C:s), 126.8 (2 C:s), 127.8, 127.9 (2 C:s), 128.2 (2 C:s), 128.6 (2 C:s), 128.7 (2 C:s), 132.5, 133.2, 139.8, 140.2, 144.6, 165.4, 168.4. Anal calc for C₂₆H₂₇ClN₂O₅, C, 64.7; H, 5.6; N, 5.8. Found: C, 64.6; H, 5.6; N, 5.8.

### (−)-N-[1-(4-Chlorophenyl)-3-dimethylamino-propyl]-4-phenyl-benzamide oxalate (−)-(5o)

[α]_{D} −19.0 (c 0.36, MeOH)

### (+)-N-[1-(4-Chlorophenyl)-3-dimethylamino-propyl]-4-phenyl-benzamide oxalate (+)-(5o)

[α]_{D} +20.8 (c 0.21, MeOH)

### N-[1-(4-Chlorophenyl)-3-dimethylaminopropyl]-4-phenoxy-benzamide oxalate (5p)

Reaction of 4-phenoxy-benzoic acid (100 mg, 0.47 mmol) with 2a yielded 150 mg pure product (78%) which was converted to the oxalate salt. Mp 112.5-113.6° C. ¹H NMR (CD₃OD) δ 2.25-2.42 (m, 2H), 2.86 (s, 6H), 3.11-3.29 (m, 2H), 5.17 (dd, 1H, J=5.5, 10.2 Hz), 6.98 (d, 2H, J=8.8 Hz), 7.02 (dd, 2H, J=1.1, 8.8 Hz), 7.18, (tt, 1H, J=1.1, 7.3 Hz), 7.34-7.43 (m, 6H), 7.87 (d, 2H, J=8.8 Hz). ¹³C NMR (CD₃OD) δ 30.2, 42.2 (2 C:s), 50.9, 55.2, 117.1 (2 C:s), 119.6 (2 C:s), 124.2, 128.1 (2 C:s), 128.5 (2 C:s), 129.4 (2 C:s), 129.7 (2 C:s), 133.1, 140.3, 155.9, 161.0, 165.8, 167.9, Anal. Calcd for C₂₆H₂₇ClN₂O₆: C, 62.6; H, 5.5; N, 5.6. Found: C, 62.5; H, 5.4; N, 5.6.

### N-[1-(4-Chlorophenyl)-3-dimethylaminopropyl]-4-trifluoromethyl-benzamide oxalate (5q)

Reaction of 4-trifluoromethyl-benzoic acid (150 mg, 0.78 mmol) with 2a yielded 120 mg pure product (66%) which was converted to the oxalate salt obtained as an yellow oil. ¹H NMR (CD₃OD) δ 2.28-2.45 (m, 2H), 2.89 (s, 6H), 3.16-3.33 (m, 2H), 5.21 (dd, 1H, J=5.5, 9.5 Hz), 7.36 (d, 2H, J=8.4 Hz), 7.45 (d, 2H, J=8.4 Hz), 7.75 (d, 2H, J=8.1 Hz), 8.03 (d, 2H, J=8.1 Hz). ¹³C NMR (CD₃OD) δ 30.3, 42.4 (2 C:s), 51.2, 55.2, 124.2 (q, ¹J_{CF}=389.5 Hz), 125.3 (q, 2 C:s, ³J_{CF}=4.6 Hz), 128.1 (2 C:s), 128.2 (2 C:s), 128.6 (2 C:s), 132.9 (q, ²J_{CF}=33.7 Hz), 133.4, 137.6, 140.1, 163.4, 167.3. Anal calc for C₂₁H₂₂F₃ClN₂O₅×H₂O, C, 51.1; H, 4.9; N, 5.7. Found: C, 50.8; H, 4.5; N, 5.5.

### N-[1-(4-Chlorophenyl)-3-dimethylaminopropyl]-2-phenyl-acetamide oxalate (5r)

Compound 2a (0.1 g, 0.47 mmol) was dissolved in THF (20 mL). Phenylacetic acid (0.07 g, 0.5 mmol), EDC (0.1 g, 0.5 mmol) and DMAP (6 mg, 0.05 mmol) were added and the mixture was stirred for three days. Saturated aqueous NaHCO₃ (30 mL) and EtOAc (20 mL) were added. The phases were separated and the water phase extracted with EtOAc. The combined organic phases were washed (water, brine) and evaporated. The resulting mixture was purified with flash chromatography using MeOH/CH₂Cl₂/NEt₃ (5/94.9/0.1) to give 0.1 g (60%) of the title compound which was converted to the oxalate salt. Mp 140.3-141.2° C. ¹H NMR (CD₃OD) δ 2.17-2.21 (m, 2H), 2.81 (s, 6H), 2.98-3.07 (m, 2H), 3.55 (s, 2H), 4.95 (dd, 1H, J=5.5, 9.5 Hz), 7.20-7.36 (m, 9H). ¹³C NMR (CD₃OD) δ 30.4, 42.2 (2 C:s), 42.5, 50.3, 55.1, 126.7, 128.1 (2 C:s), 128.3 (2 C:s), 128.6 (2 C:s), 128.8 (2 C:s), 133.3, 135.6, 139.6, 165.4, 172.6. Anal calc for C₂₁H₂₅ClN₂O₅, C, 59.9; H, 6.0; N, 6.7. Found: C, 60.1; H, 5.9; N, 6.5.

### N-[1-(4-Chlorophenyl)-3-dimethylaminopropyl]-(R)-2-methoxy-2-phenyl acetamide oxalate (5s)

Compound 2a (0.9 g, 4.2 mmol) was dissolved in THF (100 mL). (r)-2-Methoxy-2-phenyl-acetic acid (0.7 g, 4.2 mmol), EDC (0.9 g, 4.5 mmol) and DMAP (0.12 g, 0.9 mmol) were added and the mixture was stirred for three days. Saturated aqueous NaHCO₃ (100 mL) and EtOAc (100 mL) were added. The phases were separated and the water phase extracted with EtOAc. The combined organic phases were washed (water, brine) and evaporated. The resulting mixture was purified with flash chromatography using MeOH/CH₂Cl₂/NEt₃ (5/94.9/0.1) to give 1.1 g (67%) of the pure diastereomeric mixture which was separated by repeated flash chromatography using MeOH/CH₂Cl₂/NEt₃ (5/94.9/0.1) until the pure diastereomers were obtained (>99.7%).

### (+)-N-[1-Chlorophenyl)-3-dimethylaminopropyl]-(R)-2-methoxy-2-phenyl-acetamide oxalate ((+)-5s)

Mp 179.2-181.0. [α]_{D} +50.0 (c 0.034, MeOH)

¹H NMR (CD₃OD) δ 2.21-2.28 (m, 2H), 2.77 (s, 6H), 2.93-2.97 (m, 2H), 3.34 (s, 3H), 4.69 (s, 1H), 4.98 (dd, 1H, J=6.2, 8.8 Hz), 7.30-7.40 (m, 7H), 7.46 (dd, 2H, J=1.1, 8.1 Hz). ¹³C NMR (CD₃OD) δ 30.0, 42.3 (2 C:s), 49.8, 55.1, 56.1, 83.6, 126.9 (2 C:s), 128.1 (2 C:s), 128.3 (2 C:s), 128.5, 128.7 (2 C:s), 133.3, 137.5, 139.6, 164.5, 172.2. Anal. Calcd. for C₂₂H₂₇ClN₂O₆: C, 58.6; H, 6.0; N, 6.2. Found: C, 58.8; H, 6.1; N, 6.1.

### (−)-N-[1-(4-Chlorophenyl)-3-dimethylaminopropyl]-(R)-2-methoxy-2-phenyl-acetamide oxalate ((−)-5s)

Mp 163.0-163.8. [α]_{D} −111.4 (c 0.044, MeOH

¹H NMR (CD₃OD) δ 2.19-2.34 (m, 2H), 2.82 (s, 6H), 3.00-3.09 (m, 2H), 3.34 (s, 3H), 4.71 (s, 1H), 4.97 (dd, 1H, J=5.8, 9.5 Hz), 7.27-7.38 (m, 9H). ¹³C NMR (CD₂OD) δ 29.8, 42.2 (2 C:s), 50.0, 55.2, 55.9, 83.5, 127.0 (2 C:s), 128.0 (2 C:s), 128.2, 128.4 (2 C:s), 128.6 (2 C:s), 133.3, 137.3, 139.6, 165.4, 172.0. Anal. Calcd for C₂₂H₂₇ClN₂O₆: C, 58.6; H, 6.0; N, 6.2. Found: C, 58.5; H, 6.0; N, 6.1.

General Method for the Synthesis of Sulphonamides 6a-6c

NEt₃ (0.15 mL, 1 mmol) and the sulphonylchloride were added to a solution of 2a (100 mg, 0.47 mmol) in THF (25 mL), and the reaction was stirred at rt for 18 h. Saturated aqueous NaHCO₃ (25 mL) was added and the mixture was extracted twice with EtOAc. The combined organic phases were washed (water and brine) and concentrated to afford the title compounds, which were converted to the corresponding oxalate salt.

### N-[1-(4-Chlorophenyl)-3-dimethylaminopropyl]-4-methyl-benzenesulfonamide oxalate (6a)

p-Tosyl chloride (94 mg, 0.47 mmol) and 2a yielded 190 mg (quant) pure product, which was converted to the oxalate salt. Mp 191.1-192.0° C. ¹H NMR (CD₃OD) δ 1.95-2.15 (m, 2H), 2.33 (s, 3H), 2.71 (s, 6H), 2.86-2.93 (m, 1H), 3.04-3.12 (m, 1H), 4.34 (dd, 1H, J=5.5, 9.5 Hz), 7.01 (d, 2H, J=8.4 Hz), 7.08 (d, 2H, J=8.4 Hz), 7.12 (d, 2H, J=8.3 Hz), 7.45 (d, 2H, J=8.3 Hz). ¹³C NMR (CD₃OD) δ 20.0, 32.1, 42.6 (2 C:s), 55.2, 55.4, 126.7 (2 C:s), 128.0 (2 C:s), 128.2 (2 C:s), 129.0 (2 C:s), 132.9, 138.2, 139.1, 143.2, 169.7. Anal. Calcd for C₂₀H₂₅ClN₂O₆S: C, 52.6; H, 5.5; N, 6.1. Found: C, 52.6; H, 5.6; N, 6.1.

### N-[1-(4-Chlorophenyl)-3-dimethylaminopropyl]-4-phenyl-benzenesulfonamide oxalate (6b)

4-Phenyl-benzene-sulphonyl chloride (119 mg, 0.47 mmol) and 2a yielded 200 mg (quant) pure product, which was converted to the oxalate salt. Mp 198.0-198.6° C. ¹H NMR (CD₃OD) δ 2.04-2.13 (m, 1H), 2.17-2.27 (m, 1H), 2.86 (s, 6H), 3.09-3.16 (m, 1H), 3.26-3.32 (m, 1H), 4.45 (dd, 1H, J=5.5, 9.5 Hz), 7.05 (d, 2H, J=8.8 Hz), 7.08 (d, 2H, J=8.8 Hz), 7.37 (tt, 1H, J=1.5, 7.7 Hz), 7.44 (td, 2H, J=7.7, 8.4 Hz), 7.51 (d, 2H, J=8.4 Hz), 7.56 (dt, 2H, J=1.5, 8.4 Hz), 7.63 (d, 2H, J=8.4 Hz). ¹³C NMR (CD₃OD) δ 31.7, 42.3, 42.5, 55.2, 55.4, 127.0 (2 C:s), 127.1 (2 C:s), 127.3 (2 C:s), 128.1, 128.2 (2 C:s), 128.3 (2 C:s), 128.7 (2 C:s), 133.2, 138.6, 139.3, 139.5, 145.3, 161.3. Anal. Calcd for C₂₅H₂₇ClN₂O₆S×1.5H₂O: C, 55.0; H, 5.5; N, 5.1. Found: C, 55.3; H, 5.2; N, 5.5.

### N-[1-(4-Chlorophenyl)-3-dimethylaminopropyl]-2-naphthyl-benzenesulfonamide oxalate (6c)

2-Naphthyl-sulphonyl chloride (106 mg, 0.47 mmol) and 2a yielded 190 mg (quant) pure product, which was converted to the oxalate salt. Mp 246.5-248.1° C. ¹H NMR (CD₃OD) δ 1.99-2.08 (m, 1H), 2.16-2.26 (m, 1H), 2.83 (s, 6H), 3.07-3.12 (m, 1H), 3.23-3.31 (m, 1H), 4.47 (dd, 1H, J=5.5, 9.5 Hz), 6.84 (d, 2H, J=8.1 Hz), 7.00 (d, 2H, J=8.1 Hz), 7.55-7.65 (m, 3H), 7.78-7.89 (m, 3H), 8.01 (s, 1H). ¹³C NMR (CD₃OD) δ 31.7, 42.3, 42.5, 51.2, 55.4, 121.9, 127.3, 127.5, 127.8, 127.9 (2 C:s), 128.0 (2 C:s), 128.5, 128.7, 128.9, 131.9, 133.1, 134.6, 137.7, 138.3, 160.1. Anal. Calcd for C₂₃H₂₅ClN₂O₆S×2 H₂O: C, 52.2; H, 5.5; N, 5.3. Found: C, 52.0; H, 5.2; N, 5.6.

General Method for the Synthesis of Carbamates 7a-e and Ureas 8a-e

NEt₃ (0.15 mL, 1 mmol) and the appropriate isocyanate (0.5 mmol) were added to a solution of 1a or 2a (100 mg, 0.47 mmol) in THF (25 mL), and the reaction was stirred at rt for 18 h. The reaction mixture was concentrated and purified using flash chromatography (first CH₂Cl₂ 100%, thereafter a gradient up to 50% MeOH). The fractions containing product were pooled and evaporated and the residue converted to the corresponding oxalate salt.

### N-[1-(4-Chlorophenyl)-3-dimethylaminopropyloxycarbonyl]-2-methylphenyl-amine (7a)

2-Methylphenyl-isocyanate (62 mg) and 1a yielded 160 mg (98%) of the title compound, which was converted to the oxalate salt. Mp 134.0-135.7° C. ¹H NMR (CD₃OD) δ 2.20 (s, 3H), 2.22-2.37 (m, 2H), 2.86 (s, 6H), 3.18-3.30 (m, 2H), 5.76 (dd, 1H, J=3.6, 8.0 Hz), 7.04-7.18 (m, 3H), 7.32-7.42 (m, 5H). ¹³C NMR (CD₃OD) δ 16.7, 31.2, 42.3 (2 C:s), 54.3, 73.2, 124.4, 125.5, 126.1, 127.6 (2 C:s), 128.6 (2 C:s), 130.2, 132.0, 133.8, 135.6, 138.8, 154.5, 165.4. Anal. Calcd for C₂₁H₂₅ClN₂O₆: C, 57.7; H, 5.8; N, 6.4. Found: C, 57.8; H, 5.7; N, 6.5.

### N-[1-(4-Chlorophenyl)-3-dimethylaminopropyloxycarbonyl]-3-methoxyphenyl-amine (7b)

3-Methoxyphenyl-isocyanate (70 mg) and 1a yielded 100 mg (57%) of the title compound, which was converted to the oxalate salt. Mp 191.9-193.2° C. ¹H NMR (CD₃OD) δ 2.19-2.38 (m, 2H), 2.87 (s, 6H), 3.18-3.24 (m, 2H), 3.74 (s, 3H), 5.77 (dd, 1H, J=4.4, 8.8 Hz), 6.58 (dd, 1H, J=2.2, 8.4), 6.93 (dd, 1H, J=1.1, 8.0 Hz), 7.09-7.17 (m, 2H), 7.38 (d, 2H, J=8.8 Hz), 7.42 (d, 2H, J=8.8 Hz). ¹³C NMR (CD₃OD) δ 31.2, 42.3 (2 C:s), 54.3, 54.4, 72.8, 104.3, 108.4, 110.7, 127.5 (2 C:s), 128.6 (2 C:s), 129.3, 133.9, 138.6, 139.7, 153.1, 160.3, 165.4. Anal. Calcd for C₂₁H₂₅ClN₂O₇: C, 55.7; H, 5.6; N, 6.2. Found: C, 55.8; H, 5.5; N, 6.1.

### N-[1-(4-Chlorophenyl)-3-dimethylaminopropyloxycarbonyl]-4-tert-butylphenyl-amine (7c)

4-tert-Butylphenyl-isocyanate (88 mg) and 1a yielded 180 mg (99%) of the title compound, which was converted to the oxalate salt isolated as a colorless oil. ¹H NMR (CD₃OD) δ 1.26 (s, 9H), 2.20-2.40 (m, 2H), 2.86 (s, 6H), 3.18-3.27 (m, 2H), 5.77 (dd, 1H, J=4.4, 8.8 Hz), 7.28 (d, 2H, J=8.8 Hz), 7.32 (d, 2H, J=8.8 Hz), 7.37 (d, 2H, J=8.8 Hz), 7.42 (d, 2H, J=8.8 Hz). ¹³C (CD₃OD) δ 30.5 (3 C:s), 31.3, 33.7, 42.3 (2 C:s), 54.4, 72.7, 118.4 (2 C:s), 125.3 (2 C:s), 127.5 (2 C:s), 128.6 (2 C:s), 133.8, 135.8, 138.8, 146.0, 153.4, 165.0. Anal. Calcd for C₂₄H₃₁ClN₂O₆: C, 60.2; H, 6.5; N, 5.8. Found: C, 60.1; H, 6.6; N, 5.7.

### N-[1-(4-Chlorophenyl)-3-dimethylaminopropyloxycarbonyl]-4-phenoxyphenyl-amine (7d)

4-Phenoxyphenyl-isocyanate (100 mg) and 1a yielded 130 mg (64%) of the title compound, which was converted to the oxalate salt isolated as a colorless oil. ¹H NMR (CD₃OD) δ 2.22-2.41 (m, 2H), 2.88 (s, 6H), 3.17-3.28 (m, 2H), 5.78 (dd, 1H, J=4.4, 8.4 Hz), 6.89 (d, 2H, J=8.8 Hz), 6.92 (d, 2H, J=8.8 Hz), 7.04 (tt, 1H, J=1.2, 7.3 Hz), 7.28 (dd, 2H, J=7.3, 9.4 Hz), 7.37-7;44 (m, 6H). ¹³C (CD₃OD) δ 31.2, 42.3 (2 C:s), 54.4, 72.9, 117.9 (2 C:s), 119.2 (2 C:s), 120.2 (2 C:s), 122.7, 127.5 (2 C:s), 128.6 (2 C:s), 129.5 (2 C:s), 133.9, 134.2, 138.6, 152.8, 153.4, 157.8, 164.2. Anal. Calcd for C₂₆H₂₇ClN₂O₇: C, 60.6; H, 5.3; N, 5.4. Found: C, 60.6; H, 5.2; N, 5.3.

### N-[1-(4-Chlorophenyl)-3-dimethylaminopropyloxycarbonyl]-benzyl-amine (7e)

Benzyl isocyanate (66 mg) and 1a yielded 81 mg (50%) of the title compound, which was converted to the oxalate salt isolated as a colorless oil. ¹H (CD₃OD) δ 2.16-2.33 (m, 2H), 2.85 (s, 6H), 3.12-3.20 (m, 2H), 4.25 (s, 2H), 5.69 (dd, 1H, J=4.7, 8.8 Hz), 7.14-7.30 (m, 5H), 7.38 (app s, 4H). ¹³C (CD₃OD) δ 31.2, 42.2 (2 C:s), 44.2, 54.4, 72.7, 126.9, 127.0 (2 C:s), 127.4 (2 C:s), 128.1 (2 C:s), 128.5 (2 C:s), 133.7, 138.4, 138.9, 165.1, 171.3. Anal. Calcd for C₂₁H₂₅ClN₂O₆×⅓H₂O: C, 56.9; H, 5.8; N, 6.3. Found: C, 56.7; H, 5.8; N, 6.3.

### 1-[1-(4-Chlorophenyl)-3-dimethylaminopropyl]-3-(2-methylphenyl)-carbamide oxalate (8a)

2-Methylphenyl-isocyanate (67 mg) and 2a yielded 70 mg (43%) of the title compound, which was converted to the oxalate salt obtained as a yellow oil. ¹H NMR (CD₃OD) δ 2.17-2.25 (m, 2H), 2.21 (s, 3H), 2.89 (s, 6H), 3.13-3.27 (m, 2H), 6.99, (dd, 1H, J=4.0, 7.7 Hz), 7.10-7.16 (m, 3H), 7.40 (app s, 4H), 7.52 (d, 1H, J=7.7 Hz). ¹³C NMR (CD₃OD) δ 16.8, 31.4, 42.3 (2 C:s), 51.1, 55.3, 123.1, 123.9, 125.9, 127.9 (2 C:s), 128.6 (2 C:s), 129.9, 130.1, 133.0, 136.8, 141.0, 156.8, 165.3. Anal. Calcd for C₂₁H₂₆ClN₃O₅: C, 57.9; H, 6.0; N, 9.6. Found: C, 57.8; H, 6.1; N, 9.6.

### 1-[1-(4-Chlorophenyl)-3-dimethylaminopropyl]-3-(3-methoxyphenyl)-carbamide oxalate (8b)

3-Methoxyphenyl-isocyanate (74 mg) and 2a yielded 120 mg (71%) of the title compound, which was converted to the oxalate salt obtained as a yellow oil. ¹H NMR (CD₃OD) δ 2.13-2.18 (m, 2H), 2.81 (s, 6H), 3.04-3.27 (m, 2H), 3.71 (s, 3H), 4.82 (dd, 1H, J=7.3, 7.4 Hz), 6.51 (dd, 1H, J=1.8, 8.0 Hz), 6.86 (dd, 1H, J=0.8, 8.0 Hz), 7.06-7.11 (m, 2H) 7.30 (d, 2H, J=8.4 Hz) 7.37 (d, 2H, J=8.4 Hz). ¹³C NMR (CD₃OD) δ 31.6, 42.3 (2 C:s), 51.0, 54.3, 55.2, 104.6, 107.5, 111.1, 127.7 (2 C:s), 128.5 (2 C:s), 129.1, 132.9, 140.8, 141.4, 156.3, 160.2, 171.3. Anal. Calcd for C₂₁H₂₆ClN₃O₆: C, 55.8; H, 5.8; N, 9.3. Found: C, 55.8; H, 5.7; N, 9.2.

### 1-[1-(4-Chlorophenyl)-3-dimethylaminopropyl]-3-(4-tert-butylphenyl)-carbamide oxalate (8c)

4-tert-Butylphenyl-isocyanate (88 mg) and 2a yielded 150 mg (82%) of the title compound, which was converted to the oxalate salt isolated as a colorless oil. ¹H NMR (CD₃OD) δ 1.26 (s, 9H), 2.17-2.22 (m, 2H), 2.87 (s, 6H), 3.12-3.27 (m, 2H), 4.88 (dd, 1H, J=4.0, 10.6 Hz), 7.26 (app s, 4H), 7.36 (d, 2H, J=8.8 Hz), 7.39 (d, 2H, J=8.8 Hz). ¹³C NMR (CD₃OD) δ 30.46 (3 C:s), 31.4, 33.7, 42.3 (2 C:s), 50.8, 55.3, 118.8 (2 C:s), 125.2 (2 C:s), 127.7 (2 C:s), 128.6 (2 C:s), 133.1, 136.6, 140.8, 145.3, 156.5, 165.2. Anal. Calcd for C₂₄H₃₂ClN₃O₅: C, 60.3; H, 6.7; N, 8.8. Found: C, 60.2; H, 6.7; N, 8.7.

### 1-[1-(4-Chlorophenyl)-3-dimethylaminopropyl]-3-(4-phenoxyphenyl)-carbamide oxalate oxalate (8d)

4-Phenoxyphenyl-isocyanate (100 mg) and 2a yielded 150 mg (75%) of the title compound, which was converted to the oxalate salt isolated as an yellowish oil. ¹H NMR (CD₃OD) δ 2.16-2.22 (m, 2H), 2.86 (s, 6H), 3.10-3.29 (m, 2H), 4.85 (dd, 1H, J=7.0, 13.0), 6.87 (d, 2H, J=8.8 Hz), 6.90 (d, 2H, J=8.8 Hz), 7.03 (tt, 1H, J=1.2, 6.3 Hz), 7.26-7.39 (m, 8H). ¹³C NMR (CD₃OD) δ 31.5, 42.3 (2 C:s), 50.9, 55.3, 117.6 (2 C:s), 119.2 (2 C:s), 120.7 (2 C:s), 122.5, 127.8 (2 C:s), 128.6 (2 C:s), 129.4 (2 C:s), 133.0, 135.1, 141.1, 152.3, 156.5, 163.1. Anal. Calcd for C₂₆H₂₈ClN₃O₆: C, 60.7; H, 5.5; N, 8.2. Found: C, 60.6; H, 5.5; N, 8.2.

### 1-[1-(4-Chlorophenyl)-3-dimethylaminopropyl]-3-benzyl-carbamide oxalate (8e)

Benzyl isocyanate (66 mg) and 2a yielded 93 mg (57%) of the title compound, which was converted to the oxalate salt isolated as a colorless oil. ¹H NMR (CD₃OD) δ 2.12-2.28 (m, 2H), 2.83 (s, 6H), 3.01-3.22 (m, 2H), 4.26 (d, 1H, J=15.0 Hz), 4.31 (d, 1H, J=15.0 Hz), 4.79 (dd, 1H, J=2.2, 8.8 Hz), 7.19-7.30 (m, 5H), 7.35 (app s, 4H). ¹³C NMR (CD₃OD) δ 31.4, 42.2 (2 C:s), 43.4, 51.0, 55.2, 126.7, 126.8 (2 C:s), 127.8 (2 C:s), 128.1 (2 C:s), 128.5 (2 C:s), 133.1, 139.8, 140.9, 159.1, 165.2. Anal. Calcd for C₂₁H₂₆ClN₃O₅: C, 57.9; H, 6.0; N, 9.6. Found: C, 57.5; H, 6.3; N, 9.5.

General Method for the Synthesis of Carbamates 7f-i

The carboxylic acid (2.7 mmol) and NEt₃ (0.42 mL, 3 mmol) were dissolved in toluene (10 mL). Diphenylphosphorylazide (0.6 mL, 3 mmol) was added and the solution was heated to reflux for 1 h. A solution of 1a (200 mg, 0.94 mmol) in THF (5 mL) was added, and the reaction was stirred at rt for 18 h. The mixture was poured into NaOH (1M) and extracted twice with EtOAc. The combined organic phases were washed (water, brine) and concentrated. The crude oil was dissolved in CH₂Cl₂ and applied to a SAX-2 ion exchange column, washed with CH₂Cl₂ and MeOH. The product was eluted using methanolic NH₃ (2M), and concentrated. The pure products were converted to the corresponding oxalate salts for analysis, storage and biological testing.

### N-[1-(4-Chlorophenyl)-3-dimethylaminopropyloxycarbonyl]-4-phenyl-amine (7f)

4-Phenyl-benzoic acid (594 mg) and 1a yielded 280 mg (91%) of the title compound, which was converted to the oxalate salt isolated as a colorless oil. ¹H NMR (CD₃OD) δ 2.21-2.27 (m, 1H), 2.31-2.39 (m, 1H), 2.85 (s, 6H), 3.17-3.27 (m, 2H), 5.78 (dd, 1H, J=4.4, 8.8 Hz), 7.26 (tt, 1H, J=1.8, 7.4 Hz), 7.34-7.39 (m, 4H), 7.42 (d, 2H, J=8.8 Hz), 7.47-7.56 (m, 6H). ¹³C NMR (CD₃OD) δ 31.3, 42.3 (2 C:s), 54.4, 72.9, 118.8 (2 C:s), 126.2 (2 C:s), 126.7, 127.0 (2 C:s), 127.6 (2 C:s), 128.5 (2 C:s), 128.6 (2 C:s), 133.8, 136.0, 137.8, 138.7, 140.5, 153.2, 164.8. Anal. Calcd for C₂₆H₂₇ClN₂O₆: C, 62.6; H, 5.5; N, 5.6. Found: C, 62.5; H, 5.4; N, 5.5.

### N-[1-(4-Chlorophenyl)-3-dimethylaminopropyloxycarbonyl]-2-naphthyl-amine (7g)

Naphthalene-2-carboxylic acid (486 mg) and 1a yielded 180 mg (50%) of the title compound, which was converted to the oxalate salt isolated as a yellow oil. ¹H NMR (CD₃OD) δ 2.14-2.35 (m, 2H), 2.76 (s, 6H), 3.01-3.21 (m, 2H), 5.77 (dd, 1H, J=4.4, 8.4 Hz), 7.22-7.38 (m, 6H), 7.50 (dd, 1H, J=2.6, 8.8 Hz), 7.65-7.72 (m, 3H), 8.00 (s, 1H). ¹³C NMR (CD₃OD) δ 31.2, 42.3 (2 C:s), 54.3, 73.0, 114.7, 119.3, 124.4, 126.2, 127.0, 127.3, 127.6 (2 C:s), 128.4, 128.6 (2 C:s), 130.2, 133.7, 134.0, 136.2, 138.8, 153.4, 166.1. Anal. Calcd for C₂₄H₂₅ClN₂O₆: C, 60.9; H, 5.3; N, 5.9. Found: C, 60.8; H, 5.3; N, 5.8.

### N-[1-(4-Chlorophenyl)-3-dimethylaminopropyloxycarbonyl]-4-methoxyphenyl-amine (7h)

4-Methoxy-benzoic acid (429 mg) and 1a yielded 140 mg (41%) of the title compound, which was converted to the oxalate salt isolated as a colorless oil. ¹H NMR (CD₃OD) δ 2.18-2.26 (m, 1H), 2.28-2.38 (m, 1H), 2.79 (s, 6H), 3.10-3.23 (m, 2H), 3.71 (s, 3H), 5.71 (dd, 1H, J=4.4, 8.8 Hz), 6.81 (d, 2H, J=8.8 Hz), 7.29 (d, 2H, J=8.8 Hz), 7.33 (d, 2H, J=8.1 Hz), 7.38 (d, 2H, J=8.1 Hz). ¹³C NMR (CD₃OD) δ 31.2, 42.3 (2 C:s), 54.2, 54.5, 72.9, 113.8 (2 C:s), 120.3 (2 C:s), 127.4 (2 C:s), 128.6 (2 C:s), 131.5, 133.7, 138.9, 153.6, 155.9, 168.8. Anal. Calcd for C₂₁H₂₅ClN₂O₇: C, 55.7; H, 5.6; N, 6.2. Found: C, 55.8; H, 5.5; N, 6.1.

### N-[1-(4-Chlorophenyl)-3-dimethylaminopropyloxycarbonyl]-4-trifluoromethylphenyl-amine (7i)

4-Trifluoromethyl-benzoic acid (540 mg) and 1a yielded 110 mg (29%) of the title compound, which was converted to the oxalate salt isolated as a colorless oil. ¹H NMR (CD₃OD) δ 2.16-2.29 (m, 1H), 2.33-2.41 (m, 1H), 2.82 (s, 6H), 3.12-3.24 (m, 2H), 5.78 (dd, 1H, J=4.4, 8.8 Hz), 7.32 (d, 2H, J=8.8 Hz), 7.39 (d, 2H, J=8.8 Hz), 7.51 (d, 2H, J=8.8 Hz), 7.60 (d, 2H, J=8.8 Hz). ¹³C NMR (CD₃OD) δ 31.2, 42.3 (2 C:s), 54.2, 73.3, 118.1 (2 C:s), 125.0 (¹J_{CF}=260 Hz), 124.2 (²J_{CF}=38.9 Hz), 125.7 (2 C:s, ³J_{CF}=3.8 Hz), 127.6 (2 C:s), 128.5 (2 C:s), 133.9, 138.7, 142.4, 153.0, 168.1. Anal. Calcd for C₂₁H₂₂ClF₃N₂O₆: C, 51.4; H, 4.5; N, 5.7. Found: C, 51.4; H, 4.5; N, 5.6.

General Method for the Synthesis of Ureas 8f-i

The carboxylic acid (0.94 mmol) and NEt₃ (0.14 mL, 1 mmol) were dissolved in toluene (10 mL). Diphenylphosphorylazide (0.2 mL, 1 mmol) was added and the solution was heated to reflux for 1 h. A solution of 2a (200 mg, 0.94 mmol) in THF (5 mL) was added, and the reaction was stirred at rt for 18 h. The mixture was poured into NaOH (1M) and extracted twice with EtOAc. The combined organic phases were washed (water, brine) and concentrated. The crude oil was dissolved in CH₂Cl₂ and applied to a SAX-2 ion exchange column, washed with CH₂Cl₂ and MeOH. The product was eluted using methanolic NH₃ (2M), and concentrated. The pure products were converted to the corresponding oxalate salts for analysis, storage and biological testing.

### 1-[1-(4-Chlorophenyl)-3-dimethylaminopropyl]-3-(4-phenylphenyl)-carbamide oxalate (8f)

4-Phenyl-benzoic acid (186 mg) and 2a yielded 230 mg (60%) of the title compound, which was converted to the oxalate salt isolated as a colorless oil. ¹H NMR (CD₃OD) δ 2.21-2.30 (m, 2H), 2.88 (s, 6H), 3.15-3.28 (m, 2H), 4.90 (dd, 1H, J=6.2, 9.2 Hz), 7.26 (tt, 1H, J=2.2, 7.7 Hz), 7.35-7.42 (m, 6H), 7.45 (d, 2H, J=8.8 Hz), 7.50 (d, 2H, J=8.8 Hz), 7.54 (d, 2H, J=7.3 Hz). ¹³C NMR (CD₃OD) δ 31.4, 42.2, 42.5, 50.9, 55.3, 119.1 (2 C:s), 126.2 (2 C:s), 126.5, 126.9 (2 C:s), 127.9 (2 C:s), 128.5 (2 C:s), 128.7 (2 C:s), 133.2, 135.3, 138.8, 140.6, 140.7, 156.2, 162.3. Anal. Calcd for C₂₆H₂₈ClN₃O₅×2H₂O: C, 58.5; H, 6.0; N, 7.9. Found: C, 58.1; H, 5.5; N, 7.7.

### 1-[1-(4-Chlorophenyl)-3-dimethylaminopropyl]-3-(2-naphthyl)-carbamide oxalate (8g)

Naphtalene-2-carboxylic acid (162 mg) and 2a yielded 190 mg (53%) of the title compound, which was converted to the oxalate salt isolated as a colorless oil. ¹H NMR (CD₃OD) δ 2.12-2.21 (m, 2H), 2.80 (s, 6H), 3.09-3.17 (m, 1H), 3.19-3.26 (m, 1H), 4.89 (dd, 1H, J=3.7, 9.2 Hz), 7.25-7.39 (m, 6H), 7.45 (dd, 1H, J=2.2 Hz, 9.2 Hz), 7.65 (d, 1H, J=9.2 Hz), 7.69 (d, 2H, J=8.8 Hz), 7.94 (d, 1H, J=2.2 Hz). ¹³C NMR (CD₃OD) δ 31.3, 42.3 (2 C:s), 51.0, 55.2, 114.5, 119.8, 123.9, 126.0, 126.8, 127.2, 127.8 (2 C:s), 128.2, 128.6 (2 C:s), 129.9, 133.0, 134.2, 137.1, 141.1, 156.4, 165.6. Anal. Calcd for C₂₄H₂₆ClN₃O₅: C, 61.1; H, 5.6; N, 8.9. Found: C, 61.1; H, 5.6; N, 8.9.

### 1-[1-(4-Chlorophenyl)-3-dimethylaminopropyl]-3-(4-methoxyphenyl)-carbamide oxalate (8h)

4-Methoxy-benzoic acid (143 mg) and 2a yielded 110 mg (32%) of the title compound, which was converted to the oxalate salt isolated as a colorless oil. ¹H NMR (CD₃OD) δ 2.14-2.24 (m, 2H), 2.83 (s, 6H), 3.11-3.24 (m, 2H), 3.70 (s, 3H), 4.86 (dd, 1H, J=1.8, 8.4 Hz), 6.79 (d, 2H, J=8.8 Hz), 7.24 (d, 2H, J=8.8 Hz), 7.32 (d, 2H, J=8.4 Hz), 7.38 (d, 2H, J=8.4 Hz). ¹³C NMR (CD₃OD) δ 31.4, 42.1, 42.6, 50.9, 54.6, 55.3, 113.7 (2 C:s), 121.3 (2 C:s), 127.9 (2 C:s), 128.6 (2 C:s), 132.2, 133.0, 141.0, 155.8, 156.7, 162.8. Anal. Calcd for C₂₁H₂₆ClN₃O₆×1.5H₂O: C, 52.7; H, 6.1; N, 8.8. Found: C, 52.9; H, 5.7; N, 8.6.

### 1-[1-(4-Chlorophenyl)-3-dimethylaminopropyl]-3-(4-trifluoromethylphenyl)-carbamide oxalate (8i)

4-Trifluoromethyl-benzoic acid (180 mg) and 2a yielded 70 mg (19%) of the title compound, which was converted to the oxalate salt isolated as a colorless oil. ¹H NMR (CD₃OD) δ 2.16-2.27 (m, 2H), 2.87 (s, 6H), 3.13-3.21 (m, 1H), 3.23-3.28 (m, 1H), 4.86 (dd, 1H, J=3.7, 9.2 Hz), 7.34 (d, 2H, J=8.8 Hz), 7.39 (d, 2H, J=8.8 Hz), 7.48 (d, 2H, J=8.4 Hz), 7.56 (d, 2H, J=8.4 Hz). ¹³C NMR (CD₃OD) δ 31.3, 42.3 (2 C:s), 50.9, 55.2, 117.9 (2 C:s), 123.4 (²J_{CF}=31.4 Hz), 124.5 (¹J_{CF}=276 Hz), 125.6 (2 C:s, ³J_{CF}=5.5 Hz), 127.8 (2 C:s), 128.6 (2 C:s), 133.1, 140.9, 143.3, 155.8, 166.4. Anal. Calcd for C₂₁H₂₃ClF₃N₃O₅: C, 51.5; H, 4.7; N, 8.6. Found: C, 51.4; H, 4.6; N, 8.5.

### Example 4

### Alternative Synthesis of Amides

The appropriate amine (1 eqv, 50 mg), PS-DCC (2 eqv), PS-DMAP (0.2 eqv), carboxylic acid (5 eqv) and DCM (15 ml) were added to a vial and shaken at room temperature for four days. The mixture was then filtered and the solute was concentrated. ¹H-NMR spectra were run to control that the reactions were complete. The crude product was then dissolved in CH₂Cl₂ (20 ml) and washed with 1M NaOH (2×15 ml). The CH₂Cl₂-phase was then concentrated. ¹H-NMR spectra were run to control the purity. If the product was not pure (>98% purity) ion exchange chromatography (SCX-2) was used for the final purification. The pure product was then converted to the corresponding HCl salt using HCl saturated ether.

### N-[1-(4-Chlorophenyl)-3-dimethylamino-propyl]-2-phenylacetamide HCl (A1)

2-Phenylacetic acid (161 mg, 1.18 mmol) yielded 72 mg (A1) (92%). ¹H NMR δ 2.83-2.92 (m, 2H), 3.29 (s, 6H), 3.72-3.80 (m, 2H), 4.26 (s, 2H), 5.63 (dd, 1H, J=6.6, 14.6 Hz), 7.97-8.12 (m, 5H), 8.13-8.23 (m, 4H); ¹³C NMR δ 30.5, 42.2, 42.5 (2 C:s), 50.4, 55.2, 126.7, 128.1 (2 C:s), 128.3 (2 C:s), 128.5 (2 C:s), 128.8 (2 C:s), 133.2, 135.6, 139.8, 172.5. HRTofMS calcd for C₁₉H₂₃ClN₂O (M+) m/z 330.1499, found 330.1504.

### N-[1-(4-Chlorophenyl)-3-dimethylamino-propyl]-2-(4-trifluoromethylphenyl)acetamide HCl (A2)

2-(4-Trifluoromethylphenyl)acetic acid (250 mg, 1.18 mmol) yielded 90 mg (A2) (96%). ¹H NMR δ 2.16-2.30 (m, 1H), 2.79-2.90 (m, 1H) 2.86 (s, 6H), 3.00-3.20 (m, 2H), 3.67 (s, 2H), 4.96 (dd, 1H, J=6.2, 8.8 Hz), 7.35 (app. s, 4H), 7.48 (d, 2H, J=8.3 Hz), 7.60 (d, 2H, J=8.3 Hz); ¹³C NMR δ 30.5, 41.6, 42.1, 42.4, 51.6, 55.2, 124.4 (q, 1JCF=269.1 Hz), 125.0 (q, 2 C:s, 3JCF=3.8 Hz), 128.2 (2 C:s), 128.6 (2 C:s), 128.8 (q, 2JCF=32.1 Hz), 129.6 (2 C:s), 133.3, 139.8, 140.2, 171.5. HRTofMS calcd for C₂₀H₂₂ClF₃N₂O (M+) m/z 398.1373, found 398.1378.

### N-[1-(4-Chlorophenyl)-3-dimethylamino-propyl]-2-(4-methoxyphenyl)acetamide HCl (A3)

2-(4-Methoxyphenyl)acetic acid (196 mg, 1.18 mmol) yielded 82 mg (A3) (96%). ¹H NMR δ 2.18-2.26 (m, 2H), 2.83 (app d, 6H, J=7.3 Hz), 2.99-3.12 (m, 2H), 3.49 (s, 2H), 3.75 (s, 3H), 4.95 (t, 1H, J=7.3 Hz), 6.85 (d, 2H, J=8.4 Hz), 7.20 (d, 2H, J=8.4 Hz), 7.35 (app. s, 4H). ¹³C NMR δ 30.4, 41.6, 42.0, 42.6, 50.2, 54.4, 55.1, 113.7 (2 C:s), 127.5, 128.2 (2 C:s), 128.6 (2 C:s), 129.8 (2 C:s), 133.4, 139.5, 158.9, 173.1. HRTofMS calcd for C₂₀H₂₅ClN₂O₂ (M+) m/z 360.1605, found 360.1611.

### N-[1-(4-Chlorophenyl)-3-dimethylamino-propyl]-3-phenyl-propionamide HCl (A4)

3-Phenylpropionic acid (179 mg, 1.18 mmol) yielded 75 mg (A4) (92%). ¹H NMR δ 2.06-2.20 (m, 2H), 2.50-2.64 (m, 2H), 2.82 (s, 6H), 2.89-2.94 (m, 2H), 2.94-3.00 (m, 2H), 4.90 (dd, 1H, J=6.6, 15.0 Hz), 7.16-7.20 (m, 3H), 7.21-7.27 (m, 4H), 7.29-7.34 (m, 2H); ¹³C NMR δ 14.1, 30.3, 31.3, 37.2, 41.9, 42.7, 65.6, 126.0, 128.1 (2 C:s), 128.2 (2 C:s), 128.3 (2 C:s), 128.5 (2 C:s), 133.2, 139.6, 140.6, 173.6. HRTofMS calcd for C₂₀H₂₅ClN₂O (M+) m/z 344.1655, found 344.1659.

### N-[1-(4-Chlorophenyl)-3-dimethylamino-propyl]-3-(4-trifluoromethylphenyl) propionamide HCl (A5)

3-(4-Trifluoromethylphenyl)propanoic acid (256 mg, 1.18 mmol) yielded 81 mg (A5) (83%). ¹H NMR δ 2.10-2.18 (m, 2H), 2.52-2.67 (m, 2H), 2.84 (s, 6H), 2.96-3.30 (m, 2H), 3.32-3.40 (m, 2H), 4.92 (dd, 1H, J=4.8, 7.3 Hz), 7.20-7.26 (m, 2H), 7.32-7.38 (m, 4H), 7.48-7.54 (d, 2H, J=8.1 Hz); ¹³C NMR δ 30.0, 31.3, 32.0, 37.5, 43.1, 43.5, 64.8, 126.3 (2 C:s), 128.5 (q, 1JCF=271.4 Hz), 128.8 (q, 2JCF=29.8 Hz), 129.5 (2 C:s), 129.6 (2 C:s), 130.4 (2 C:s), 132.9, 142.8, 147.4, 172.2. HRTofMS calcd for C21H24ClF3N2O (M+) m/z 412.1529, found 412.1529.

### N-[1-(4-Chlorophenyl)-3-dimethylamino-propyl]-3-(4-methoxyphenyl)propanamide HCl (A6)

3-(4-Methoxyphenyl)propionic acid (218 mg, 1.21 mmol) yielded 80 mg (A6) (91%). ¹H NMR δ 2.06-2.16 (m, 2H), 2.47-2.60 (m, 2H), 2.76-2.84 (m, 2H), 2.82 (s, 6H), 2.91-3.02 (m, 2H), 3.70 (s, 3H), 4.88 (dd, 1H, J=6.2, 8.8 Hz), 6.78 (d, 2H, J=8.4 Hz), 7.08 (d, 2H, J=8.4 Hz), 7.20 (d, 2H, J=8.4 Hz), 7.30 (d, 2H, J=8.4 Hz). ¹³C NMR δ 30.4, 30.5, 37.5, 42.0 (2 C:s), 54.3, 55.1, 65.6, 113.6 (2 C:s), 128.0 (2 C:s), 128.5 (2 C:s), 129.3 (2 C:s), 132.5, 133.1, 139.6, 158.3, 173.7. HRTofMS calcd for C₂₁H₂₇ClN₂O₂ (M+) m/z 374.1761, found 360.1770.

### N-[1-(4-Chlorophenyl)-3-dimethylamino-propyl]-3-cinnamic amide HCl (A7)

Cinnamic acid (179 mg, 1.18 mmol) yielded 50 mg (A7) (62%). ¹H NMR δ 2.20-2.38 (m, 2H), 2.80-2.94 (m, 1H) 2.89 (s, 6H), 3.12-3.27 (m, 1H), 5.12 (dd, 1H, J=5.5, 9.2 Hz), 6.75 (d, 1H, J=15.8 Hz), 7.30-7.50 (m, 6H), 7.50-7.60 (m, 4H); ¹³C NMR (δ 14.1, 30.7, 42.1, 42.5, 65.6, 120.1, 127.6 (2 C:s), 128.2 (2 C:s), 128.7 (4 C:s), 129.7, 133.3, 134.8, 139.8, 141.3, 170.0. HRTofMS calcd for C₂₀H₂₃ClN₂O (M+) m/z 342.1499, found 342.1510.

### N-[1-(4-Chlorophenyl)-3-dimethylamino-propyl]-4-trifluoromethyl-cinnamic amide HCl (A8)

4-Trifluoromethyl-cinnamic acid (259 mg, 1.18 mmol) yielded 70 mg (A8) (97%). ¹H NMR δ 2.20-2.40 (m, 2H), 2.84-2.96 (m, 1H) 2.90 (s, 6H), 3.12-3.28 (m, 1H), 5.11 (dd, 1H, J=5.2, 13.9 Hz), 6.85 (d, 1H, J=15.8 Hz), 7.30-7.47 (m, 4H), 7.60 (d, 1H, J=15.8 Hz), 7.65-7.71 (m, 2H), 7.72-7.80 (m, 2H). HRTofMS calcd for C₂₁H₂₂ClF₃N₂O (M+) m/z 410.1373, found 410.1382.

### N-[1-(4-Chlorophenyl)-3-dimethylamino-propyl]-4-methoxy-cinnamic amide HCl (A9)

4-Methoxycinnamic acid (234 mg, 1.31 mmol) yielded 60 mg (A9) (68%). ¹H NMR δ 2.20-2.38 (m, 2H), 2.80-2.98 (m, 1H) 2.90 (s, 6H), 3.10-3.32 (m, 1H), 3.80 (s, 3H), 5.11 (dd, 1H, J=5.5, 9.2 Hz), 6.60 (d, 1H, J=15.8 Hz), 6.93 (d, 2H, J=8.4 Hz), 7.36 (d, 2H, J=8.4 Hz), 7.43 (d, 2H, J=8.4 Hz), 7.47-7.58 (m, 3H). ¹³C NMR δ 30.8, 42.1, 42.6, 50.5, 54.6, 55.2, 114.1 (2 C:s), 117.5, 127.4, 128.2 (2 C:s), 128.7 (2 C:s), 129.3 (2 C:s), 133.3, 140.0, 141.1, 161.5, 167.4. HRTofMS calcd for C₂₁H₂₅ClN₂O₂ (M+) m/Z 372.1605, found 372.1609.

### N-[1-(4-Chlorophenyl)-3-dimethylamino-propyl]-3-phenylpropiolic amide HCl (A10)

3-Phenylpropiolic acid (172 mg, 1.19 mmol) yielded 40 mg (A10) (50%). ¹H NMR δ 2.20-2.36 (m, 2H), 2.84-2.94 (m, 2H) 2.90 (s, 6H), 5.07 (dd, 1H, J=5.5, 8.8 Hz), 7.36-7.44 (m, 6H), 7.45-7.50 (m, 1H), 7.54-7.60 (m, 2H); ¹³C NMR δ 30.3, 42.3 (2 C:s), 51.0, 55.1, 82.1, 85.5, 119.9, 128.2 (2 C:s), 128.5 (2 C:s), 128.7 (2 C:s), 130.3, 132.2 (2 C:s), 133.5, 139.2, 153.9. HRTofMS calcd for C₂₀H₂₁ClN₂O (M+) m/z 340.1342, found 340.1346.

### N-[3-Dimethylamino-1-(4-methylphenyl)propyl]-2-phenylacetamide HCl (B1)

2-Phenylacetic acid (187 mg, 1.32 mmol) yielded 75 mg (B1) (93%). ¹H NMR δ 2.00-2.10 (m, 2H), 2.15 (s, 3H), 2.65 (s, 6H), 2.80-3.00 (m, 2H), 3.41 (s, 2H), 4.80 (dd, 1H, J=6.8, 9.2 Hz), 7.03 (d, 2H, J=8.0 Hz), 7.12 (d, 2H, J=8.0 Hz), 7.15-7.19 (m, 5H). ¹³C NMR δ 23.9, 34.8, 46.1, 46.6 (2 C:s), 54.7, 59.3, 130.5 (2 C:s), 130.7, 132.4 (2 C:s), 132.9 (2 C:s), 133.2 (2 C:s), 139.8, 141.5, 141.8, 176.6. HRTofMS calcd for C₂₀H₂₆N₂O (M+) m/z 310.2045, found 310.2045.

### N-[3-Dimethylamino-1-(4-methylphenyl)propyl]-2-(4-trifluoromethylphenyl)acetamide HCl (B2)

2-(4-Trifluoromethylphenyl)-acetic acid (270 mg, mmol) yielded 95 mg (B2) (98%). ¹H NMR δ 2.00-2.10 (m, 2H), 2.15 (s, 3H), 2.55 (s, 6H), 2.65-2.90 (m, 2H), 3.15 (s, 2H), 4.75 (t, 1H, J=5.2 Hz), 7.01 (d, 2H, J=8.0 Hz), 7.15 (d, 2H, J=8.0 Hz), 7.32 (d, 2H, J=8.0 Hz), 7.43 (d, 2H, J=8.0 Hz). ¹³C NMR δ 19.8, 31.2, 42.1, 42.6 (2 C:s), 51.1, 55.4, 124.5 (q, ¹J_{CF}=270.0 Hz), 125.0 (q, 2 C:s, ³J_{CF}=3.8 Hz), 126.4 (2 C:s), 128.8 (q, ²J_{CF}=32.1 Hz), 129.1 (2 C:s), 129.6 (2 C:s), 137.4, 136.0, 140.3, 171.3. HRTofMS calcd for C₂₁H₂₅F₃N₂O (M+) m/z 378.1919, found 378.1921.

### N-[3-Dimethylamino-1-(4-methylphenyl)propyl]-2-(4-methoxyphenyl)acetamide HCl (B3)

2-(4-Methoxyphenyl)acetic acid (229 mg, 1.38 mmol) yielded 82 mg (B3) (93%). ¹H NMR δ 2.03-2.08 (m, 2H), 2.13. (s, 3H), 2.65 (app. d, 6H), 2.58-3.00 (m, 2H), 3.35 (app d, 2H, J=2.4 Hz), 3.75 (s, 3H), 4.80 (dd, 1H, J=6.4, 6.4 Hz), 6.71 (d, 2H, J=8.0 Hz), 7.03 (d, 2H, J=8.0 Hz), 7.06-7.14 (m, 4H). ¹³C NMR δ 23.9, 34.8, 45.7, 46.1, 46.6, 54.7, 58.5, 59.3, 117.7 (2 C:s), 130.4 (2 C:s), 131.7, 133.2 (2 C:s), 133.9 (2 C:s), 141.5, 141.8, 162.9, 177.0. HRTofMS calcd for C₂₁H₂₈N₂O₂ (M+) m/z 340.2151, found 340.2159.

### N-[3-Dimethylamino-1-(4-methylphenyl)propyl]-3-phenylpropionamide HCl (B4)

3-Phenylpropionic acid (207 mg, 1.37 mmol) yielded 81 mg (B4) (96%). ¹H NMR δ 1.80-2.17 (m, 2H), 2.30 (s, 3H), 2.50-2.64 (m, 2H), 2.80 (s, 6H), 2.85-2.98 (m, 4H), 4.84 (dd, 1H, J=8.08, 8.08 Hz), 7.10-7.26 (m, 9H). ¹³C NMR δ 19.8, 30.6, 31.4, 37.2, 42.6, 47.1, 51.0, 55.2, 126.0, 126.3 (2 C:s), 128.2 (2 C:s), 128.3 (2 C:s), 129.1 (2 C:s), 137.3, 137.8, 140.7, 173.5. HRTofMS calcd for C₂₁H₂₈N₂O (M+) m/z 324.2202, found 324.2214.

### N-[3-Dimethylamino-1-(4-methylphenyl)propyl]-3-(4-trifluoromethylphenyl) propionamide HCl (B5)

3-(4-Trifluoromethylphenyl)propionic acid (284 mg, 1.32 mmol) yielded 80 mg (B5) (78%). ¹H NMR δ 1.88-1.96 (m, 2H), 2.30 (s, 3H), 2.50-2.66 (m, 4H), 2.82 (s, 6H), 2.94-3.02 (m, 2H), 4.80 (t, 1H, J=5.3 Hz), 7.04 (d, 2H, J=8.4 Hz), 7.34 (d, 2H, J=8.4 Hz), 7.44-7.50 (m, 4H). ¹³C NMR δ 19.8, 30.6, 31.1, 36.9 (2 C:s), 42.1, 42.6, 52.5, 125.0 (2 C:s), 126.3 (q, 2 C:s, ³J_{CF}=9.2 Hz), 127.0 (q, ¹J_{CF}=280 Hz), 128.4 (q, ²J_{CF}=33.1 Hz), 128.9 (2 C:s), 129.1 (2 C:s), 137.3, 137.9, 145.4, 173.0.

### N-[3-Dimethylamino-1-(4-methylphenyl)propyl]-3-(4-methoxyphenyl)propionamide HCl (B6)

3-(4-Methoxyphenyl)propionic acid (236 mg, 1.31 mmol) yielded 90 mg (B6) (98%). ¹H NMR δ 1.95-2.05 (m, 2H), 2.08 (s, 3H), 2.32-2.48 (m, 2H), 2.67 (s, 6H), 2.68-2.86 (m, 4H), 3.60 (s, 3H), 4.75 (t, 1H, J=9.6 Hz), 6.65 (d, 2H, J=8.8 Hz), 6.97 (d, 2H, J=8.8 Hz), 6.98-7.40 (m, 4H). ¹³C NMR δ 23.9, 34.7 (2 C:s), 41.6, 46.0, 46.7, 54.4, 58.4, 59.3, 117.6 (2 C:s), 130.4 (2 C:s), 133.1 (2 C:s), 133.3 (2 C:s), 136.6, 141.3, 141.8, 162.3, 177.7. HRTofMS calcd for C₂₂H₃₀N₂O₂ (M+) m/z 354.2307, found 354.2306.

### N-[3-Dimethylamino-1-(4-methylphenyl)propyl]cinnamic amide (B7)

Cinnamic acid (198 mg, 1.34 mmol) yielded 72 mg (B7) (86%). ¹H NMR δ 2.20-2.38 (m, 2H) 2.30 (s, 3H), 2.78-2.94 (m, 1H) 2.86 (s, 6H), 3.28-3.32 (m, 1H), 5.07 (dd, 1H, J=8.8, 14.6 Hz), 6.73 (d, 1H, J=16.8 Hz), 7.14 (d, 2H, J=7.7 Hz), 7.23-7.42 (m, 5H), 7.46-7.60 (m, 3H). ¹³C NMR δ 19.8, 30.9, 42.1 (2 C:s), 50.9, 55.3, 120.3, 126.5 (2 C:s), 127.6 (2 C:s), 128.7 (2 C:s), 129.2 (2 C:s), 129.7, 134.9, 137.6, 137.8, 141.1, 166.9. HRTofMS calcd for C₂₁H₂₆N₂O (M+) m/z 322.2045, found 322.2055.

### N-[3-Dimethylamino-1-(4-methylphenyl)propyl]-4-trifluoromethyl-cinnamic amide HCl (B8)

4-Trifluoromethyl-cinnamic acid (286 mg, mmol) yielded 60 mg (B8) (56%). ¹H NMR δ 1.90-2.08 (m, 2H), 2.20 (s, 6H), 2.30 (s, 3H), 3.28-3.34 (m, 2H), 4.96-5.02 (dd, 1H, J=7.3, 15.0 Hz), 6.78 (d, 1H, J=15.8 Hz), 7.14 (d, 2H, J=8.0 Hz), 7.22 (d, 2H, J=8.0 Hz), 7.54 (d, 1H, J=15.8 Hz), 7.65 (d, 2H, J=8.0 Hz), 7.72 (d, 2H, J=8.0 Hz). ¹³C NMR δ 19.8, 33.6, 44.2 (2 C:s), 52.0, 56.4, 123.5, 125.5 (q, 2 C:s, ³J_{CF}=8.8 Hz), 126.3 (2 C:s), 126.5 (q, ¹J_{CF}=240 Hz), 128.0 (2 C:s), 128.6 (q, ²J_{CF}=30.0 Hz), 128.9 (2 C:s), 136.9, 138.7, 138.9, 139.2, 165.7.

### N-[3-Dimethylamino-1-(4-methylphenyl)propyl]-4-methoxy-cinnamic amide HCl (B9)

4-Methoxy-cinnamic acid (232 mg, 1.30 mmol) yielded (B9) (87%). ¹H NMR δ 2.18-2.39 (m, 2H), 2.30 (s, 3H), 2.85 (s, 6H), 3.08-3.24 (m, 2H), 3.34 (s, 3H), 5.07 (dd, 1H, J=6.2, 8.8 Hz), 6.58 (d, 1H, J=15.8 Hz), 6.92 (d, 2H, J=8.8 Hz), 7.19 (d, 2H, J=8.0 Hz), 7.30 (d, 2H, J=8.0 Hz), 7.46-7.49 (m, 3H). ¹³C NMR δ 19.8, 31.0, 42.0, 42.6, 50.7, 54.6, 55.3, 114.0 (2 C:s), 117.6, 126.4 (2 C:s), 127.4, 129.2 (2 C:s), 129.3 (2 C:s), 130.0, 137.5, 140.9, 161.4, 167.2. HRTofMS calcd for C₂₂H₂₈N₂O₂ (M+) m/z 352.2151, found 352.2152.

### N-[3-Dimethylamino-1-(4-methylphenyl)propyl]-3-phenylpropiolic amide (B10)

3-Phenylpropiolic acid (193 mg, 1.32 mmol) yielded 50 mg (B10) (60%). ¹H NMR δ 2.10-2.40 (m, 2H) 2.25 (s, 3H), 2.80 (s, 6H), 3.00-3.20 (m, 2H), 5.00 (dd, 1H, J=6.2, 8.1 Hz), 7.17 (d, 2H, J=8.0 Hz), 7.27 (d, 2H, J=8.0 Hz), 7.32-7.50 (m, 3H), 7.50-7.60 (m, 2H). ¹³C NMR δ 19.8, 30.5, 41.0 (2 C:s), 51.2, 55.2, 81.0, 82.5, 120.0, 126.4 (2 C:s), 128.5 (2 C:s), 129.2 (2 C:s), 130.2, 132.2 (2 C:s), 137.0, 137.5, 154.0. HRTofMS calcd for C₂₁H₂₄N₂O (M+) m/z 320.1889, found 320.1887.

### N-[3-Dimethylamino-1-(2-naphthyl)propyl]-2-phenylacetamide HCl (C1)

2-Phenylacetic acid (148 mg, 1.18 mmol) yielded 51 mg (C1) (67%). ¹H NMR δ 2.26-2.40 (m, 2H), 2.82 (s, 6H), 3.00-3.20 (m, 2H), 3.59 (s, 2H), 5.14 (dd, 1H, J=6.2, 8.8 Hz), 7.20-7.36 (m, 5H), 7.43-7.53 (m, 3H), 7.78-7.91 (m, 4H); ¹³C NMR δ 30.5, 42.1, 42.5 (2 C:s), 51.0, 54.9, 124.4, 125.2, 125.9, 126.2, 126.7, 127.3, 127.6, 128.3 (2 C:s), 128.5, 128.8 (2 C:s), 132.8, 133.2, 135.4, 137.8, 172.6. C₂₃H₂₇ClN₂O×H₂O: C, 68.9; H, 7.3; N, 7.0. Found: C, 68.8; H, 7.3; N, 6.7.

### N-[3-Dimethylamino-1-(2-naphthyl)propyl]-2-(4-trifluoromethylphenyl)acetamide HCl (C2)

2(4-Trifluoromethylphenyl)acetic acid (223 mg, 1.18 mmol) yielded 80 mg (C2) (88%). ¹H NMR δ 2.30-2.40 (m, 2H), 2.85 (s, 6H), 3.02-3.22 (m, 2H), 3.70 (s, 2H), 5.14 (dd, 1H, J=7.3, 15.0 Hz), 7.42-7.54 (m, 5H), 7.56-7.64 (d, 2H, J=8.0 Hz), 7.76-7.92 (m, 4H); ¹³C NMR δ 30.5, 41.8, 42.1, 42.5, 51.2, 55.0, 124.2 (q, 1JCF=269.1 Hz), 124.4, 125 (q, 2 C:s, 3JCF=3.8 Hz), 125.2, 126.0, 126.2, 127.4, 127.6, 128.5, 128.8 (q, 2JCF=32.1 Hz), 129.6 (2 C:s), 133.1, 133.4, 137.9, 140.2, 171.6. Anal. Calcd. for C₂₄H₂₆ClF₃N₂O×H₂O: C, 61.5; H, 6.0; N, 6.0. Found: C, 61.7; H, 6.0; N, 5.6.

### N-[3-Dimethylamino-1-(2-naphthyl)propyl]-2-(4-methoxyphenyl)acetamide HCl (C3)

2-(4-Methoxyphenyl)acetic acid (182 mg, 1.10 mmol) yielded 65 mg (C3) (79%). ¹H NMR δ 2.05-2.23 (m, 2H), 2.43 (s, 6H), 2.58-2.60 (m, 2H), 3.49 (s, 2H), 3.75 (s, 3H), 5.09 (dd, 1H, J=6.24, 8.44 Hz), 6.85 (d, 2H, J=8.8 Hz), 7.22 (d, 2H, J=8.8 Hz), 7.40-7.49 (m, 3H), 7.77-7.84 (m, 4H). ¹³C NMR δ 32.0, 41.8, 43.3 (2 C:s), 54.4, 55.8, 65.6, 113.7 (2 C:s), 124.4, 124.9, 125.7, 126.0, 127.3, 127.6, 127.7, 128.2, 129.8 (2 C:s), 132.9, 133.5, 138.9, 158.9, 172.8. HRTofMS calcd for C₂₄H₂₈N₂O₂ (M+) m/z 376.2151, found 376.2165.

### N-[3-Dimethylamino-1-(2-naphthyl)propyl]-3-phenyl-propionamide HCl (C4)

3-Phenylpropionic acid (164 mg, 1.20 mmol) yielded 52 mg (C4) (66%). ¹H NMR δ 2.10-2.30 (m, 2H), 2.52-2.68 (m, 2H), 2.83 (s, 6H), 2.93 (t, 2H, J=7.7 Hz), 2.99 (t, 2H, J=7.7 Hz), 5.10 (dd, 1H, J=5.9, 9.5 Hz), 7.10-7.23 (m, 6H), 7.38 (dd, 1H, J=1.8, 8.8 Hz), 7.45-7.53 (m, 2H), 7.80-7.90 (m, 3H); ¹³C NMR δ 30.4, 31.4, 37.2, 41.9, 42.6, 50.7, 55.4, 124.4, 125.2, 125.9, 126.0, 126.1, 127.3, 127.7, 128.2 (2 C:s), 128.3 (2 C:s), 128.4, 133.0, 133.4, 138.0, 140.7, 173.9. HRTofMS calcd for C₂₄H₂₈N₂O (M+) m/z 360.2202, found 360.2210.

### N-[3-Dimethylamino-1-(2-naphthyl)propyl]-2-(4-trifluoromethylphenyl)propionamide HCl (C5)

3-(4-Trifluoromethylphenyl)propionic acid (239 mg, 1.17 mmol) yielded 79 mg (C5) (84%). ¹H NMR δ 2.20-2.35 (m, 2H), 2.56-2.60 (m, 2H), 2.79-2.80 (m, 1H) 2.84 (s, 6H), 2.96-3.02 (m, 2H), 3.03-3.20 (m, 1H), 5.11 (dd, 1H, J=7.3, 14.3 Hz), 7.29-7.40 (m, 2H), 7.41-7.54 (m, 4H), 7.74-7.93 (m, 5H); ¹³C NMR δ 30.4, 31.1, 36.7, 42.1, 42.3, 50.9, 55.3, 124.3, 124.9 (q, 2 C:s, 3JCF=3.8 Hz), 125.3, 125.9, 126.2, 126.8 (q, 1JCF=241.5 Hz), 127.4, 127.7, 128.5 (q, 2JCF=35.3 Hz), 128.9 (2 C:s), 133.0, 133.4, 138.1, 145.4, 173.2. Anal. Calcd. for C₂₅H₂₈ClF₃N₂O—HCl×H₂O: C, 62.2; H, 6.3; N, 5.8. Found: C, 62.3; H, 6.2; N, 5.6.

### N-[3-Dimethylamino-1-(2-naphthyl)propyl]-3-(4-methoxyphenyl-propion amide HCl (C6)

3-(4-Methoxyphenyl)propionic acid (220 mg, 1.22 mmol) yielded 80 mg (C6) (93%). ¹H NMR δ 2.10-2.22 (m, 2H), 2.48-2.61 (m, 2H), 2.61-2.70 (m, 1H) 2.65 (s, 6H), 2.76-2.89 (m, 3H), 3.66 (s, 3H), 5.07 (dd, 1H, J=6.2, 8.4 Hz), 6.71 (d, 2H, J=8.4 Hz), 7.06 (d, 2H, J=8.4 Hz), 7.34-7.39 (dd, 1H, J=1.8, 8.4 Hz), 7.42-7.50 (m, 2H) 7.71 (s, 1H), 7.78-7.86 (m, 3H). ¹³C NMR δ 30.6, 31.2, 37.6, 42.7 (2 C:s), 50.9, 54.3, 55.5, 113.5 (2 C:s), 124.5, 125.0, 125.8, 126.0, 127.3, 127.7, 128.3, 129.2 (2 C:s), 132.5, 133.0, 133.5, 138.5, 158.2, 173.7. HRTofMS calcd for C₂₅H₃₀N₂O₂ (M+) m/z 390.2307, found 390.2316.

### N-[3-Dimethylamino-1-(2-naphthyl)propyl]-3-cinnamic amide HCl (C7)

Cinnamic acid (162 mg, 1.17 mmol) yielded 60 mg (C7) (76%). ¹H NMR δ 2.37-2.45 (m, 2H), 2.88-2.95 (m, 1H) 2.91 (s, 6H), 3.13-3.29 (m, 1H), 5.30 (t, 1H, J=7.0 Hz), 6.75 (d, 1H, J=15.8 Hz), 7.34-7.42 (m, 3H), 7.45-7.52 (m, 2H), 7.53-7.59 (m, 4H), 7.82-7.95 (m, 4H); ¹³C NMR δ 30.7, 42.1 (2 C:s), 55.4, 65.6, 120.0, 124.4, 125.3, 126.0, 126.2, 127.4, 127.6 (2 C:s), 127.7, 128.6, 128.7 (2 C:s), 129.7, 133.0, 133.4, 134.8, 138.0, 141.4, 167.0. HRTofMS calcd for C₂₄H₂₆N₂O (M+) m/z 358.2045, found 358.2045.

### N-[3-Dimethylamino-1-(2-naphthyl)propyl]-4-trifluoromethyl-cinnamic amide HCl (C8)

4-Trifluoromethylcinnamic acid (237 mg, 1.20 mmol) yielded 55 mg (C8) (59%). ¹H NMR δ 2.37-2.50 (m, 2H), 2.84-2.96 (m, 1H) 2.90 (s, 6H), 3.14-3.22 (m, 1H), 5.30 (dd, 1H, J=6.6, 14.7 Hz), 6.90 (d, 1H, J=15.8 Hz), 7.44-7.53 (m, 2H), 7.54-7.62 (m, 1H), 7.63-7.71 (m, 2H), 7.72-7.79 (m, 3H), 7.82-7.96 (m, 4H); ¹³C NMR δ 30.7, 42.4 (2 C:s), 51.2, 55.1, 123.1, 124.2 (q, 1JCF=279.1 Hz), 124.4, 125.4, 125.5 (q, 2 C:s, 3JCF=3.8 Hz), 126.0, 126.2, 127.4, 127.7, 128.1 (2 C:s), 128.6, 130.9 (q, 2JCF=34.3 Hz), 133.1, 133.5, 138.0, 138.7, 139.3, 166.4. HRTofMS calcd for C₂₅H₂₅F₃N₂O (M+) m/z 426.1919, found 426.1922.

### N-[3-Dimethylamino-1-(2-naphthyl)propyl]-4-methoxy-cinnamic amide HCl (C9)

4-Methoxycinnamic acid (220 mg, 1.23 mmol) yielded 40 mg (C9) (47%). ¹H NMR δ 2.36-2.45 (m, 2H), 2.85-2.94 (m, 1H) 2.91 (s, 6H), 3.10-3.30 (m, 1H), 3.80 (s, 3H), 5.29 (dd, 1H, J=7.32, 15.0 Hz), 6.60 (d, 1H, J=7.0 Hz), 6.88-6.98 (m, 2H), 7.44-7.60 (m, 6H), 7.80-7.96 (m, 4H). ¹³C NMR δ 30.8, 42.0, 42.5, 54.5, 55.3, 88.0, 114.1 (2 C:s), 117.1, 124.1, 125.0, 125.8, 126.0, 127.4 (2 C:s), 127.7, 128.0, 129.3 (2 C:s), 133.0, 133.2, 138.0, 141.0, 161.1, 167.0. HRTofMS calcd for C₂₅H₂₈N₂O₂ (M+) m/z 388.2151, found 388.2162.

### N-[3-Dimethylamino-1-(2-naphthyl)propyl]-3-phenylpropiolic amide HCl (C10)

3-Phenylpropiolic acid (161 mg, 1.19 mmol) yielded 50 mg (C10) (64%). ¹H NMR δ 2.32-2.48 (m, 2H), 2.80-2.94 (m, 1H) 2.88 (s, 6H), 3.12-3.22 (m, 1H), 5.25 (t, 1H, J=8.4 Hz), 7.36-7.43 (m, 2H), 7.44-7.51 (m, 2H), 7.53-7.60 (m, 3H), 7.81-7.95 (m, 5H); ¹³C NMR δ 30.0, 42.0, 52.0, 54.5, 65.0, 82.0, 85.0, 120.0, 124.0, 125.0, 126.0, 126.1, 127.0, 127.1, 129.0 (2 C:s), 130.0, 131.5 (2 C:s), 133.0, 133.1, 137.5, 152.4, 154.0. HRTofMS calcd for C₂₄H₂₄N₂O (M+) m/z 356.1889, found 356.1887.

Table 1 shows the results from the synthesis of a thirty-membered amide library using the alternative method for synthesizing the amides.

TABLE 1
SYNTHESIS OF A THIRTY MEMBERED LIBRARY

| | | |
|---|---|---|
| 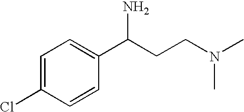 | 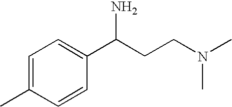 | 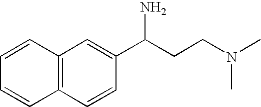 |
| A | B | C |
| 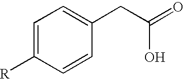 | 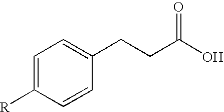 | 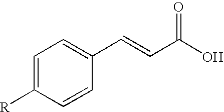 |
| 1. R = H | 4. R = H | 7. R = H |
| 2. R = CF₃ | 5. R = CF₃ | 8. R = CF₃ |
| 3. R = OMe | 6. R = OMe | 9. R = OMe |

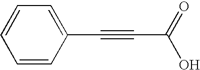

10

| | amine | | | | | | | | | | | |
|---|---|---|---|---|---|---|---|---|---|---|---|---|
| | A | | | | B | | | | C | | | |
| | | | Ion | | | | Ion | | | | Ion | |
| | Extraction | | exchange | | Extraction | | exchange | | Extraction | | exchange | |
| | Yield | Purity | Yield | Purity | Yield | Purity | Yield | Purity | Yield | Purity | Yield | Purity |
| acid | % | % | % | % | % | % | % | % | % | % | % | % |
| 1 | 92 | 100 | — | — | 93 | 100 | — | — | 67 | 100 | — | — |
| 2 | 96 | 100 | — | — | 98 | 100 | — | — | 88 | 100 | — | — |
| 3 | 96 | 100 | — | — | 93 | 100 | — | — | 79 | 100 | — | — |
| 4 | 92 | 100 | — | — | 96 | 100 | — | — | 66 | 100 | — | — |
| 5 | 83 | 100 | — | — | 78 | 100 | — | — | 84 | 100 | — | — |
| 6 | 91 | 100 | — | — | 98 | 100 | — | — | 94 | 100 | — | — |
| 7 | 87 | 85 | 62 | 100 | 132 | 93 | 86 | 100 | 76 | 100 | — | — |
| 8 | 97 | 100 | — | — | 56 | 100 | — | — | 200 | 51 | 59 | 100 |
| 9 | 83 | 97 | 68 | 100 | 87 | 100 | — | — | 118 | 88 | 47 | 100 |
| 10 | 147 | 88 | 46 | 100 | 120 | 75 | 60 | 100 | 102 | 76 | 64 | 100 |

All reactions were run to 100% conversion according to ¹H NMR spectroscopy. Purities were determined by ¹H NMR spectroscopy. All yields >100% are mainly due to remaining carboxylic acid in the sample.

### Example 5

### Miniaturization of the Alternative Synthesis of Amides

To check the robustness of the alternative method and to enable the production of larger libraries, the reaction was scaled down linearly from 50 mg to 25 and 5 mg of the starting amine, respectively. Table 2 is a summary of the miniaturization experiments. As seen in Table 2, this was accomplished without problems as both the yields and purities were in the same range as for the 50 mg reactions.

TABLE 2
MINIATURIZATION EXPERIMENTS

| | Scale | Yield | Purity | Scale | Yield | Purity |
|---|---|---|---|---|---|---|
| Cmpd | (mg) | % | % | (mg) | % | % |
| (A1) | 25 | 94 | 100 | 5 | 92 | 100 |
| (A7) | 25 | 83 | 100 | 5 | 97 | 100 |

All reactions were run to 100% conversion according to ¹H NMR spectroscopy. Purities were determined by ¹H NMR spectroscopy after basic extraction.

### Example 6

### Receptor Selection and Amplification (R-SAT) Assays

The functional receptor assay, Receptor Selection and Amplification Technology (R-SAT™), was used (with minor modifications from the procedure described previously (Brann, M. R. U.S. Pat. No. 5,707,798, 1998; Chem. Abstr. 1998, 128, 111548) to screen compounds for efficacy at the UII receptor. The RSAT assay was conducted as described herein.

Method 1

Receptor Selection and Amplification (R-SAT) Assays

Briefly, NIH3T3 cells were grown in tissue culture treated rollerbottles to 40-80% confluence in Dulbecco's modified Eagle's medium (DMEM) supplemented with 10% bovine calf serum (Hyclone) and 1% penicillin/streptomycin/glutamine (Invitrogen). Cells were transfected for 12-18 hours with the human urotensin II receptor and the β-galactosidase marker. After transfection, the cells were trypsinized, harvested and frozen in DMEM containing 10% DMSO. Frozen cells were later thawed and tested for a response to a control compound to perform quality control before initiation of pharmacological testing, ensuring the correct pharmacological response and sufficient sensitivity. To initiate the pharmacological assay, cells were thawed rapidly and prepared in DMEM media containing 0.4% calf serum (Hyclone), 30% UltraCulture (Biowhittaker), and 1% penicillin/streptomycin/glutamine (Invitrogen), and then plated at 10,000-40,000 cells per well of a 96½ area plate that contained either the test compounds or reference ligands. Cells were then grown in a humidified atmosphere with 5% ambient CO₂ for five days. Media was then removed from the plates and marker gene activity was measured by the addition of the beta-galactosidase substrate ONPG (in PBS with 5% NP-40). The resulting colorimetric reaction was measured in a spectrophotometric plate reader (Titertek Inc.) at 420 nM.

pEC50 represents the negative logarithm of the concentration of ligand that caused 50% activation of the basal receptor response. Percent activation was calculated as the difference between the absorbance measurements in the absence of added ligand compared with that in the presence of saturating concentrations of ligand normalized to the absorbance difference for the reference ligand, which was assigned a value of 100%.

These experiments provide a molecular profile, or fingerprint, for each of these agents at the human UII receptor. Table 3 shows the results of the RSAT assay for compounds 4a-4q, 5a-5s, 6a, 7a-7i, and 8a-8i. Table 4 shows the results of the RSAT assay for compounds A1-A10, B1-B10, and C1-C10. As can be seen in Tables 3 and 4, the compounds are agonists at the UII receptor.

TABLE 3
RSAT ASSAY RESULTS FOR COMPOUNDS 4a-4q,
5a-5s, 6a, 7a-7i, and 8a-8i.

| Cmpd | pEC₅₀ | Efficacy |
|---|---|---|
| 4a | 5.77 ± 0.01 | 126 ± 34 |
| 4b | 5.89 ± 0.05 | 128 ± 31 |
| 4c | 5.56 ± 0.01 | 56 ± 6 |
| 4d | 5.90 ± 0.12 | 94 ± 42 |
| 4e | 5.68 ± 0.02 | 120 ± 57 |
| 4f | 5.95 ± 0.05 | 118 ± 37 |
| 4g | 5.97 ± 0.24 | 95 ± 37 |
| 4h | 5.73 ± 0.15 | 86 ± 0 |
| 4i | 6.33 ± 0.6 | 60 ± 4 |
| 4j | 5.60 ± 0.05 | 119 ± 44 |
| 4k | 5.64 ± 0.02 | 95 ± 24 |
| 4l | 5.68 ± 0.04 | 49 ± 28 |
| 4m | 6.28 ± 0.14 | 53 ± 27 |
| 4n | 5.72 ± 0.04 | 104 ± 34 |
| 4o | 5.79 ± 0.1 | 115 ± 71 |
| 4p | 5.43-5.81 | 52-101 |
| 4q | 5.68-5.93 | 36-38 |
| 5a | 5.85 ± 0.01 | 158 ± 39 |
| 5b | 5.45 ± 0.04 | 175 ± 15 |
| 5c | 5.99 ± 0.01 | 140 ± 14 |
| 5d | 5.87 ± 0.2 | 148 ± 33 |
| 5e | 5.87 ± 0.17 | 145 ± 25 |
| 5f | 5.44 ± 0.08 | 159 ± 1 |
| 5g | 5.37 ± 0.14 | 179 ± 11 |
| 5h | 5.76 ± 0.13 | 142 ± 14 |
| 5i | 5.81 ± 0.06 | 147 ± 29 |
| 5j | 5.54 ± 0.04 | 164 ± 20 |
| 5k | 5.29 ± 0.09 | 105 ± 4 |
| 5l | 5.67 ± 0.17 | 148 ± 32 |
| 5m | 5.75 ± 0.22 | 152 ± 20 |
| 5n | 6.39 ± 0.19 | 109 ± 18 |
| 5o | 7.11 ± 0.01 | 116 ± 11 |
| (−) 5o | 5.92-5.94 | 112-114 |
| (+) 5o | 7.56-7.91 | 104-108 |
| 5p | 7.18 ± 0.2 | 91 ± 17 |
| 5q | 6.99-7.18 | 100-127 |
| 5r | 5.71 ± 0.16 | 154 ± 34 |
| (+) 5s | 5.30 ± 0.07 | 112 ± 22 |
| (−) 5s | 6.06 ± 0.03 | 165 ± 10 |
| 6a | 5.19 ± 0.09 | 74 ± 10 |
| 7a | 5.41 ± 0.07 | 170 ± 21 |
| 7b | 5.95 ± 0.09 | 133 ± 36 |
| 7c | 5.75 ± 0.57 | 105 ± 8 |
| 7d | 6.90 | 96 |
| 7e | 5.67 ± 0.11 | 180 ± 8 |
| 7f | 6.44-6.67 | 43-75 |
| 7g | 6.12-6.58 | 74-107 |
| 7h | 5.61-6.02 | 130-223 |
| 7i | 6.51-6.75 | 70-114 |
| 8a | 5.64 ± 0.15 | 166 ± 24 |
| 8b | 5.87 ± 0.17 | 147 ± 26 |
| 8c | 6.73 ± 0.33 | 101 ± 36 |
| 8d | 6.84-7.20 | 102-119 |
| 8e | 5.64-5.75 | 166-193 |
| 8f | 6.75-6.96 | 75-111 |
| 8g | 6.42-6.64 | 89-129 |
| 8h | 5.63-5.74 | 176-231 |
| 8i | 6.81-7.01 | 93-120 |

Results were determined in R-SAT assays and are expressed as pEC₅₀, the negative of the log EC₅₀ in molarity. Results are the average±standard deviation of 2-X determinations of the EC₅₀ where each compound was tested in eight doses in triplicate.

TABLE 4
RSAT ASSAY RESULTS FOR COMPOUNDS A1-A10, B1-B10, and C1-C10

| | | |
|---|---|---|
| 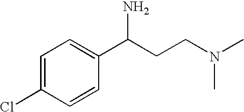 | 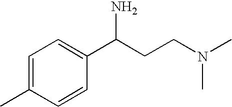 | 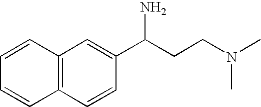 |
| A | B | C |
| 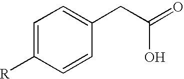 | 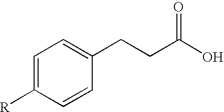 | 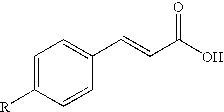 |
| 1. R = H | 4. R = H | 7. R = H |
| 2. R = CF₃ | 5. R = CF₃ | 8. R = CF₃ |
| 3. R = OMe | 6. R = OMe | 9. R = OMe |

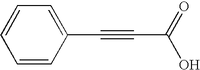

10

| cmpd | | |
|---|---|---|
| A | B | C |

| | ₚEC₅₀^{a} | Efficacy^{b} | ₚEC₅₀^{a} | Efficacy^{b} | ₚEC₅₀^{a} | Efficacy^{b} |
|---|---|---|---|---|---|---|
| 1 | 5.73 ± 0.47 | 76 ± 5 | 5.38 ± 0.07 | 83 ± 11 | 5.76 ± 0.17 | 121 ± 31 |
| 2 | 6.06 ± 0.23 | 53 ± 7 | 5.95 ± 0.03 | 25 ± 4 | 6.24 ± 0.06 | 40 ± 3 |
| 3 | 5.82 ± 0.21 | 65 ± 18 | 5.23 ± 0.05 | 62 ± 5 | NA^{c} | NA^{c} |
| 4 | 5.78 ± 0.10 | 139 ± 5 | 5.34 ± 0.14 | 73 ± 9 | 6.22 ± 0.20 | 107 ± 22 |
| 5 | 5.95 ± 0.31 | 73 ± 10 | 5.64 ± 0.09 | 60 ± 6 | 6.43 ± 0.07 | 75 ± 1 |
| 6 | 5.53 ± 0.03 | 101 ± 12 | 5.27 ± 0.02 | 67 ± 14 | 5.85 ± 0.11 | 113 ± 9 |
| 7 | 6.37 ± 0.12 | 128 ± 10 | 5.81 ± 0.07 | 97 ± 6 | 6.23 ± 0.18 | 116 ± 3 |
| 8 | 6.89 ± 0.06 | 133 ± 3 | 6.36 ± 0.08 | 96 ± 1 | 6.87 ± 0 | 117 ± 1 |
| 9 | 6.29 ± 0.04 | 99 ± 4 | 5.70 ± 0.08 | 79 ± 14 | 6.42 ± 0.06 | 106 ± 16 |
| 10 | 6.40 ± 0.04 | 124 ± 6 | 5.51 ± 0.07 | 131 ± 0 | 6.34 ± 0.05 | 115 ± 14 |

^{a}Results were determined in R-SAT assays and are expressed as pEC₅₀, the negative of the log EC₅₀ in molarity. Results are the average ± standard deviations of 2-5 determinations of the EC₅₀ where each compound was tested in eight doses in triplicate.
^{b}The % efficacy values are normalized to UII at 100%.
^{c}NA = No detectable activity

### Example 7

### Structural Activity Relationships

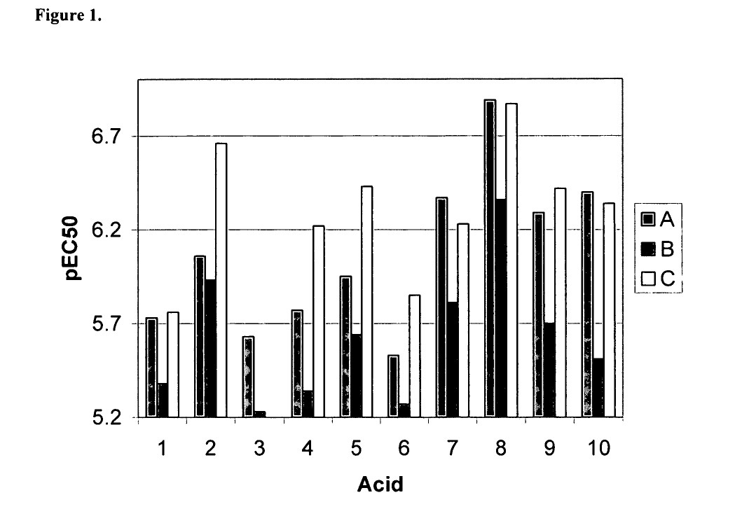

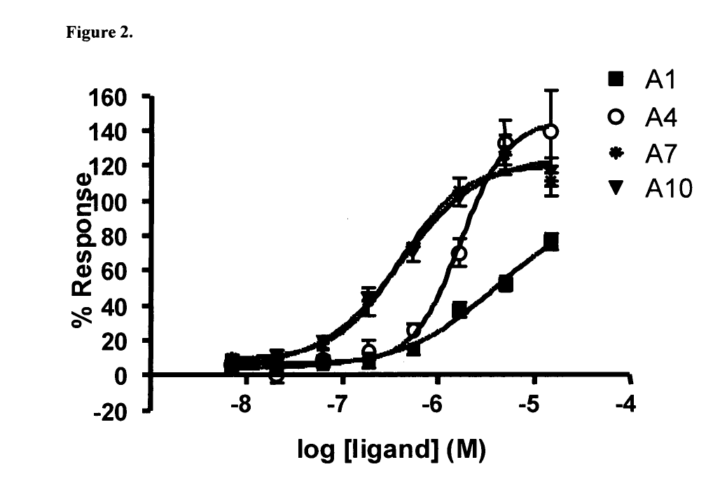

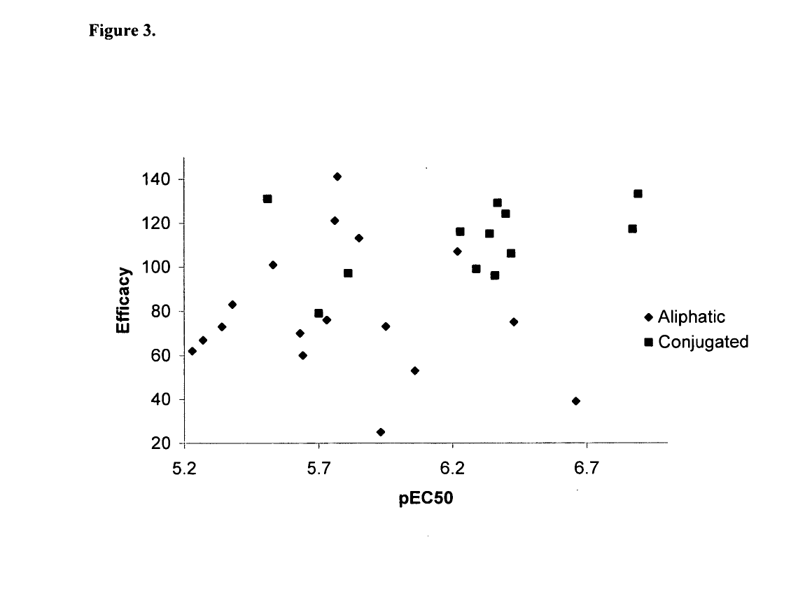

To investigate the effect of substituents on the aromatic ring of the amine moiety, several UII agonists compounds were explored using the RSAT assay as described above. FIG. 1 is a bar graph of the UII-receptor agonist potencies of the synthesized amides divided into families. FIG. 2 is a graph of the UII receptor activity of A1, A4, A7 and A 10 in the functional cell based R-SAT assay. FIG. 3 is a graph of the scatter plot of the correlation between efficacy and pEC₅₀ values for aliphatic [A1-C6] (diamonds) and conjugated derivatives [A7-C10] (squares).

As shown in FIG. 1, the A and C series showed higher potencies than the corresponding B derivatives. These results indicate that an electron deficient 4-Cl-phenyl or a sterically demanding 2-naphthyl system is more beneficial than an electron-rich 4-Me-phenyl system. Another trend in FIG. 1 is that the 4-CF₃-phenyl substituent (in 2, 5 and 8) is more favorable for potency than phenyl (1, 4 and 7) or 4-OMe-phenyl (3, 6 and 9) substitution in the respective series (A-C). When comparing the amides in the series A1, A4, A7, and 10, as seen in FIG. 2, the trend is that potency increases in the order 2-phenylacetic≈3-phenylpropanoic<cinnamic≈2-propiolic acid. This is also shown in FIG. 3, where the conjugated amides have both higher potencies and efficacies as compared to the aliphatic derivatives.

What is claimed is:

1. A compound of Formula (I): 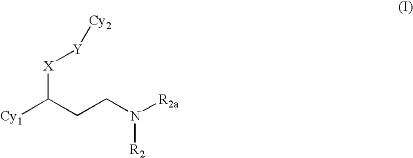
as a single isomer, a mixture of isomers, or a as a racemic mixture of isomers;
as a solvate or a pharmaceutically acceptable salt thereof, wherein:
X is selected from the group consisting of: C₁-C₄alkylene, C₁-C₄alkenylene, C₁-C₄alkynylene, —N(R₁)—, and —O—;
Y is selected from the group consisting of: C₁-C₄alkylene, C₁-C₄alkenylene, C₁-C₄alkynylene, —C(═O)—, —C(═O)N(R₁)—, —S(O)₂—, —S(O)—, —S(O)₂N(R₁)—, —S(O)N(R₁)—, —N(R₁)—: —C(═O)O—, —C(═O)O—W—, —C(═O)W—, —C(═O)CH(OR₁)—, —C(═O)N(R₁)—, —C(═O)N(R₁)W—, —S(O)₂W—, —S(O)W—, —S(O)₂N(R₁)W—, —S(O)N(R₁)W— and —N(R₁)W—;
W is selected from the group consisting of: C₁-C₄alkylene, C₁-C₄alkenylene, and C₁-C₄alkynylene;
R₁, R₁ₐ and R_{1b} are each independently selected from the group consisting of hydrogen, alkyl, alkenyl, alkynyl, cycloalkyl, cycloalkenyl, aryl, heteroaryl, and heteroalicyclyl;
Cy₁ and Cy₂ are each independently selected from the group consisting of aryl and heteroaryl;
R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ can be taken together to form a C₂-C₁₀ heteroalicyclyl; and
Z is Oxygen or Sulfur.

2. The compound of claim 1, wherein Cy₁ and Cy₂ are each independently selected from the group consisting of: 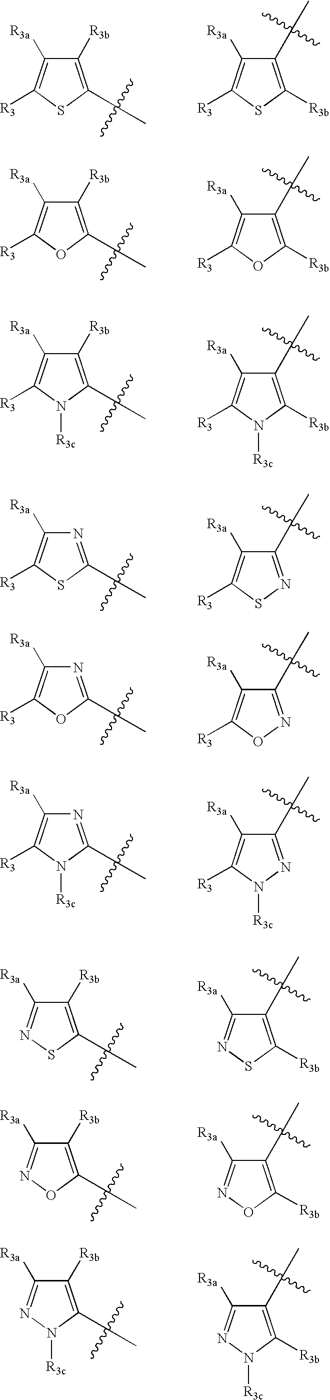 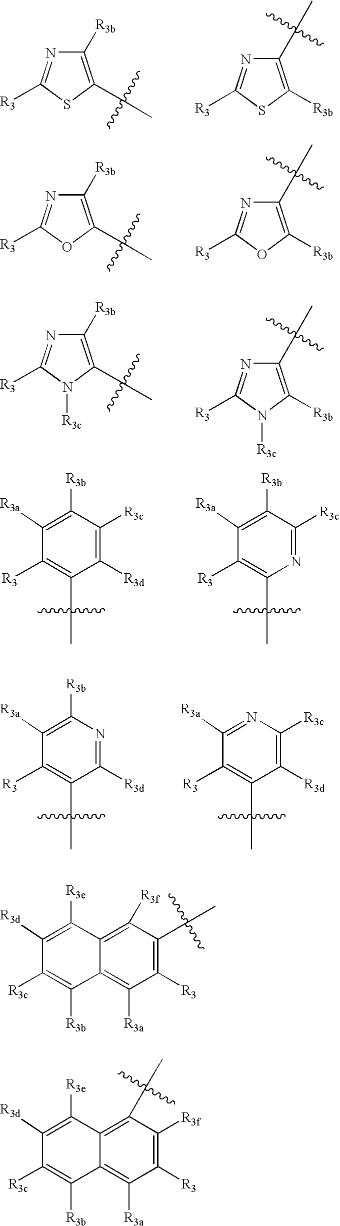
wherein R₃, R₃ₐ, R_{3b}, R_{3c}, R_{3d}, R₃ₑ and R_{3f} are each independently selected from the group consisting of alkyl, alkenyl, alkynyl, cycloalkyl, cycloalkenyl, aryl, heteroaryl, heteroalicyclyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁R₁ₐ, —C(R₁)═NR₁ₐ, NR₁R₁ₐ, —N═CR₁R₁ₐ, —N(R₁)—C(=Z)R₁ₐ, —N(R₁)—C(=Z)NR₁ₐR_{1b}, —S(O)NR₁, R₁ₐ, S(O)₂NR₁R₁ₐ, —N(R₁)—S(═O)R₁ₐ, —N(R₁)—S(═O)₂R₁ₐ, —OR₁, —SR₁, and —OC(=Z)R₁; or
if two R groups selected from the group consisting of R₃, R₃ₐ, R_{3b}, R_{3c}, R_{3d}, R₃ₑ, and R_{3f} are covalently bonded to adjacent atoms, then the two R groups can be taken together to form a cycloalkyl, aryl, heteroaryl or heteroalicyclyl group.

3. The compound of claim 1, wherein:
X is —N(R₁)—;
Y is selected from the group consisting of C₁-C₄alkylene, C₁-C₄alkenylene, C₁-C₄alkynylene, —C(═O)—, —C(═O)N(R₁)—, —S(O)₂—, —S(O)—, —S(O)₂N(R₁)—, —S(O)N(R₁)—, N(R₁)—: —C(═O)O—, —C(═O)O—W—, —C(═O)W—, —C(═O)CH(OR₁)—, —C(═O)N(R₁)—, —C(═O)N(R₁)W—, —S(O)₂W—, —S(O)W—, —S(O)₂N(R₁)W—, —S(O)N(R₁)W— and —N(R₁)W—;
Cy₁ and Cy₂ are each independently selected from the group consisting of aryl and heteroaryl; and
R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ can be taken together to form a C₂-C₁₀ heteroalicyclyl.

4. The compound of claim 1, wherein:
X is —N(R₁)—;
Y is —C(═O)—;
Cy₁ and Cy₂ are each independently selected from the group consisting of aryl and heteroaryl; and
R₂ and R₂a are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ can be taken together to form a C₂-C₁₀ heteroalicyclyl.

5. The compound of claim 1, wherein:
X is —N(R₁)—;
Y is —C(═O)—;
Cy₁ and Cy₂ are aryls; and
R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ may be taken together to form a C₂-C₁₀ heteroalicyclyl.

6. The compound of claim 1, wherein:
X is —N(R₁)—;
Y is —C(═O)—;
Cy₁ and Cy₂ are p-substituted aryls; and
R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ may be taken together to form a C₂-C₁₀ heteroalicyclyl.

7. The compound of claim 1, wherein:
X is —N(R₁)—;
Y is —C(═O)—;
Cy₁ is a p-substituted aryl substituted with a halogen;
Cy₂ is a p-substituted aryl substituted with an aryl; and
R₂ and R₂ₐ are each independently selected from the group consisting of alkyl, alkenyl, alkynyl, cycloalkyl, cycloalkenyl, heteroalicyclyl, haloalkyl, haloalkoxy, and aralkyl; or R₂ and R₂ₐ may be taken together to form a C₂-C₁₀ heteroalicyclyl.

8. The compound of claim 1, wherein:
X is —N(R₁)—;
Y is —C(═O)—;
Cy₁ is a p-substituted aryl substituted with a halogen;
Cy₂ is a p-substituted aryl substituted with an aryl; and
R₂ and R₂ₐ are alkyl groups.

9. A compound of claim 1, wherein the compound is selected from the group consisting of:
[3-(4-Chlorophenyl)-3-(4-methylbenzyloxypropyl]-N,N-dimethyl amine (3a); and
[3-(4-Chlorophenyl)-3-(2-methoxybenzyloxypropyl]-N,N-dimethyl amine (3b);
as a single isomer, a mixture of isomers, or a as a racemic mixture of isomers; as a solvate or polymorph; or as metabolite or a pharmaceutically acceptable salt or prodrug thereof.

10. A compound of claim 1, wherein the compound is selected from the group consisting of:
1-(4-Chlorophenyl)-3-dimethylamino-propyl 2-methyl-benzoate HCl (4a);
1-(4-Chlorophenyl)-3-dimethylamino-propyl 2-ethyl-benzoate HCl (4b);
1-(4-Chlorophenyl)-3-dimethylamino-propyl 4-methyl-benzoate HCl (4c);
1-(4-Chlorophenyl)-3-dimethylamino-propyl 2,3-dimethyl-benzoate HCl (4d);
1-(4-Chlorophenyl)-3-dimethylamino-propyl 3-methoxy-2-methyl-benzoate HCl (4e);
1-(4-Chlorophenyl)-3-dimethylamino-propyl 3-chloro-2-methyl-benzoate HCl (4f);
1-(4-Chlorophenyl)-3-dimethylamino-propyl 4-bromo-2-methyl-benzoate HCl (4g);
1-(4-Chlorophenyl)-3-dimethylamino-propyl 2,5-dimethyl-benzoate HCl (4h);
1-(4-Chlorophenyl)-3-dimethylamino-propyl 2,4,5-trimethyl-benzoate HCl (4i);
1-(4-Chlorophenyl)-3-dimethylamino-propyl 3-methyl-thiophene-2-carboxylate HCl (4j);
1-(4-Chlorophenyl)-3-dimethylamino-propyl naphthalene-1-carboxylate HCl (4k);
1-(4-Chlorophenyl)-3-dimethylamino-propyl naphthalene-2-carboxylate HCl (4l);
1-(4-Chlorophenyl)-3-dimethylamino-propyl 4-phenyl-benzoate HCl (4m);
1-(4-Chlorophenyl)-3-dimethylamino-propyl 1-methyl-indole-2-carboxylate HCl (4n);
1-(4-Chlorophenyl)-3-dimethylamino-propyl 1-methyl-indole-3-carboxylate HCl (4o);
1-(4-Chlorophenyl)-3-dimethylamino-propyl 4-methoxy-benzoate oxalate (4p); and
1-(4-Chlorophenyl)-3-dimethylamino-propyl 4-trifluoromethyl-benzoate oxalate (4q);
as a single isomer, a mixture of isomers, or a as a racemic mixture of isomers; as a solvate or polymorph; or as metabolite or a pharmaceutically acceptable salt or prodrug thereof.

11. A compound of claim 1, wherein the compound is selected from the group consisting of:
N-[1-(4-Chlorophenyl)-3-dimethylamino-propyl]-benzamide HCl (5a);
N-[1-(4-Chlorophenyl)-3-dimethylamino-propyl]-2-methyl-benzamide oxalate (5b);
N-[1-(4-Chlorophenyl)-3-dimethylamino-propyl]-2-ethyl-benzamide oxalate (5c)
N-[1-(4-Chlorophenyl)-3-dimethylaminopropyl]-4-methoxy-benzamide oxalate (5d);
N-[1-(4-Chlorophenyl)-3-dimethylaminopropyl]-4-dimethylamino-benzamide oxalate (5e);
N-[1-(4-Chlorophenyl)-3-dimethylamino-propyl]-2,3-dimethyl-benzamide oxalate (5f);
N-[1-(4-Chlorophenyl)-3-dimethylamino-propyl]-3-methoxy-2-methyl-benzamide oxalate (5g);
N-[1-(4-Chlorophenyl)-3-dimethylamino-propyl]-3-chloro-2-methyl-benzamide oxalate (5h);
N-[1-(4-Chlorophenyl)-3-dimethylamino-propyl]-2,4-dimethyl-benzamide oxalate (5i);
N-[1-(4-Chlorophenyl)-3-dimethylamino-propyl]-2,5-dimethyl-benzamide oxalate (5j);
N-[1-(4-Chlorophenyl)-3-dimethylamino-propyl]-6-chloro-2-methyl-benzamide oxalate (5k);
N-(1-(4-chlorophenyl)-3-(dimethylamino)propyl)benzo[d][1,3]dioxole-5-carboxamide oxalate (5l);
N-[1-(4-Chlorophenyl)-3-dimethylaminopropyl]-2,4,5-trimethyl-benzamide oxalate (5m);
N-[1-(4-Chlorophenyl)-3-dimethylamino-propyl]-2-naphthyl-carboxamide oxalate (5n);
N-[1-(4-Chlorophenyl)-3-dimethylamino-propyl]-4-phenyl-benzamide oxalate (5o);
(−)-N-[1-(4-Chlorophenyl)-3-dimethylamino-propyl]-4-phenyl-benzamide oxalate ((−)-5o);
(+)-N-[1-(4-Chlorophenyl)-3-dimethylamino-propyl]-4-phenyl-benzamide oxalate ((+)-5o);
N-[1-(4-Chlorophenyl)-3-dimethylaminopropyl]-4-phenoxy-benzamide oxalate (5p);
N-[1-(4-Chlorophenyl)-3-dimethylaminopropyl]-4-trifluoromethyl-benzamide oxalate (5q);
N-[1-(4-Chlorophenyl)-3-dimethylaminopropyl]-2-phenyl-acetamide oxalate (5r);
N-[1-(4-Chlorophenyl)-3-dimethylaminopropyl]-(R)-2-methoxy-2-phenyl acetamide oxalate (5s);
(+)-N-[1-(4-Chlorophenyl)-3-dimethylaminopropyl]-(R)-2-methoxy-2-phenylacetamide oxalate ((+)-5s); and
(−)-N-[1-(4-Chlorophenyl)-3-dimethylaminopropyl]-(R)-2-methoxy-2-phenylacetamide oxalate ((−)-5s);
as a single isomer, a mixture of isomers, or a as a racemic mixture of isomers; as a solvate or polymorph; or as metabolite or a pharmaceutically acceptable salt or prodrug thereof.

12. A compound of claim 1, wherein the compound is selected from the group consisting of:
N-[1-(4-Chlorophenyl)-3-dimethylaminopropyl]-4-methyl-benzenesulfonamide oxalate (6a);
N-[1-(4-Chlorophenyl)-3-dimethylaminopropyl]-4-phenyl-benzenesulfonamide oxalate (6b); and
N-[1-(4-Chlorophenyl)-3-dimethylaminopropyl]-2-naphthyl-benzenesulfonamide oxalate (6c);
as a single isomer, a mixture of isomers, or a as a racemic mixture of isomers; as a solvate or polymorph; or as metabolite or a pharmaceutically acceptable salt or prodrug thereof.

13. A compound of claim 1, wherein the compound is selected from the group consisting of:
N-[1-(4-Chlorophenyl)-3-dimethylaminopropyloxycarbonyl]-2-methylphenyl-amine (7a);
N-[1-(4-Chlorophenyl)-3-dimethylaminopropyloxycarbonyl]-3-methoxyphenyl-amine (7b);
N-[1-(4-Chlorophenyl)-3-dimethylaminopropyloxycarbonyl]-4-tert-butylphenyl-amine (7c);
N-[1-(4-Chlorophenyl)-3-dimethylaminopropyloxycarbonyl]-4-phenoxyphenyl-amine (7d); and
N-[1-(4-Chlorophenyl)-3-dimethylaminopropyloxycarbonyl]-benzyl-amine (7e);
as a single isomer, a mixture of isomers, or a as a racemic mixture of isomers; as a solvate or polymorph; or as metabolite or a pharmaceutically acceptable salt or prodrug thereof.

14. A compound of claim 1, wherein the compound is selected from the group consisting of:
N-[1-(4-Chlorophenyl)-3-dimethylaminopropyloxycarbonyl]-4-phenyl-amine (7f);
N-[1-(4-Chlorophenyl)-3-dimethylaminopropyloxycarbonyl]-2-naphthyl-amine (7g);
N-[1-(4-Chlorophenyl)-3-dimethylaminopropyloxycarbonyl]-4-methoxyphenyl-amine (7h); and
N-[1-(4-Chlorophenyl)-3-dimethylaminopropyloxycarbonyl]-4-trifluoromethylphenyl-amine (7i);
as a single isomer, a mixture of isomers, or a as a racemic mixture of isomers; as a solvate or polymorph; or as metabolite or a pharmaceutically acceptable salt or prodrug thereof.

15. A compound of claim 1, wherein the compound is selected from the group consisting of:
1-[1-(4-Chlorophenyl)-3-dimethylaminopropyl]-3-(2-methylphenyl)-carbamide oxalate (8a);
1-[1-(4-Chlorophenyl)-3-dimethylaminopropyl]-3-(4-tert-butylphenyl)-carbamide oxalate (8c);
1-[1-(4-Chlorophenyl)-3-dimethylaminopropyl]-3-(4-phenoxyphenyl)-carbamide oxalate (8d); and
1-[1-(4-Chlorophenyl)-3-dimethylaminopropyl]-3-benzyl-carbamide oxalate (8e);
as a single isomer, a mixture of isomers, or a as a racemic mixture of isomers; as a solvate or polymorph; or as metabolite or a pharmaceutically acceptable salt or prodrug thereof.

16. A compound of claim 1, wherein the compound is selected from the group consisting of:
1-[1-(4-Chlorophenyl)-3-dimethylaminopropyl]-3-(4-phenylphenyl)-carbamide oxalate (8f);
1-[1-(4-Chlorophenyl)-3-dimethylaminopropyl]-3-(2-naphthyl)carbamide oxalate (8g);
1-[1-(4-Chlorophenyl)-3-dimethylaminopropyl]-3-(4-methoxyphenyl)-carbamide oxalate (8h); and
1-[1-(4-Chlorophenyl)-3-dimethylaminopropyl]-3-(4-trifluoromethylphenyl)-carbamide oxalate (8i);
as a single isomer, a mixture of isomers, or a as a racemic mixture of isomers; as a solvate or polymorph; or as metabolite or a pharmaceutically acceptable salt or prodrug thereof.

17. A compound of claim 1, wherein the compound is selected from the group consisting of:
N-[1-(4-Chlorophenyl)-3-dimethylamino-propyl]-2-phenylacetamide HCl (A1);
N-[1-(4-Chlorophenyl)-3-dimethylamino-propyl]-2-(4-trifluoromethylphenyl)acetamide HCl (A2);
N-[1-(4-Chlorophenyl)-3-dimethylamino-propyl]-2-(4-methoxyphenyl)acetamide HCl (A3);
N-[1-(4-Chlorophenyl)-3-dimethylamino-propyl]-3-phenyl-propionamide HCl (A4);
N-[1-(4-Chlorophenyl)-3-dimethylamino-propyl]-3-(4-trifluoromethylphenyl) propionamide HCl (A5);
N-[1-(4-Chlorophenyl)-3-dimethylamino-propyl]-3-(4-methoxyphenyl)propanamide HCl (A6);
N-[1-(4-Chlorophenyl)-3-dimethylamino-propyl]-3-cinnamic amide HCl (A7);
N-[1-(4-Chlorophenyl)-3-dimethylamino-propyl]-4-trifluoromethyl-cinnamic amide HCl (A8);
N-[1-(4-Chlorophenyl)-3-dimethylamino-propyl]-4-methoxy-cinnamic amide HCl (A9); and
N-[1-(4-Chlorophenyl)-3-dimethylamino-propyl]-3-phenylpropiolic amide HCl (A10);
as a single isomer, a mixture of isomers, or a as a racemic mixture of isomers; as a solvate or polymorph; or as metabolite or a pharmaceutically acceptable salt or prodrug thereof.

18. A compound of claim 1, wherein the compound is selected from the group consisting of:
N-[3-Dimethylamino-1-(4-methylphenyl)propyl]-2-phenylacetamide HCl (B1);
N-[3-Dimethylamino-1-(4-methylphenyl)propyl]-2-(4-trifluoromethylphenyl)acetamide HCl (B2);
N-[3-Dimethylamino-1-(4-methylphenyl)propyl]-2-(4-methoxyphenyl)acetamide HCl (B3);
N-[3-Dimethylamino-1-(4-methylphenyl)propyl]-3-phenylpropionamide HCl (B4);
N-[3-Dimethylamino-1-(4-methylphenyl)propyl]-3-(4-trifluoromethylphenyl) propionamide HCl (B5);
N-[3-Dimethylamino-1-(4-methylphenyl)propyl]-3-(4-methoxyphenyl)propionamide HCl (B6);
N-[3-Dimethylamino-1-(4-methylphenyl)propyl]cinnamic amide (B7);
N-[3-Dimethylamino-1-(4-methylphenyl)propyl]-4-trifluoromethyl-cinnamic amide HCl (B8);
N-[3-Dimethylamino-1-(4-methylphenyl)propyl]-4-methoxy-cinnamic amide HCl (B9); and
N-[3-Dimethylamino-1-(4-methylphenyl)propyl]-3-phenylpropiolic amide (B10);
as a single isomer, a mixture of isomers, or a as a racemic mixture of isomers; as a solvate or polymorph; or as metabolite or a pharmaceutically acceptable salt or prodrug thereof.

19. A compound of claim 1, wherein the compound is selected from the group consisting of:
N-[3-Dimethylamino-1-(2-naphthyl)propyl]-2-phenylacetamide HCl (C1);
N-[3-Dimethylamino-1-(2-naphthyl)propyl]-2-(4-trifluoromethylphenyl)acetamide HCl (C2);
N-[3-Dimethylamino-1-(2-naphthyl)propyl]-2-(4-methoxyphenyl)acetamide HCl (C3);
N-[3-Dimethylamino-1-(2-naphthyl)propyl]-3-phenyl-propionamide HCl (C4);
N-[3-Dimethylamino-1-(2-naphthyl)propyl]-2-(4-trifluoromethylphenyl)propionamide HCl (C5);
N-[3-Dimethylamino-1-(2-naphthyl)propyl]-3-(4-methoxyphenyl-propion amide HCl (C6);
N-[3-Dimethylamino-1-(2-naphthyl)propyl]-3-cinnamic amide HCl (C7);
N-[3-Dimethylamino-1-(2-naphthyl)propyl]-4-trifluoromethyl-cinnamic amide HCl (C8);
N-[3-Dimethylamino-1-(2-naphthyl)propyl]-4-methoxy-cinnamic amide HCl (C9); and
N-[3-Dimethylamino-1-(2-naphthyl)propyl]-3-phenylpropiolic amide HCl (C10);
as a single isomer, a mixture of isomers, or a as a racemic mixture of isomers; as a solvate or polymorph; or as metabolite or a pharmaceutically acceptable salt or prodrug thereof.

20. A polymorph, ester, metabolite or prodrug of the compound of claim 1.

21. A pharmaceutical composition, comprising a pharmaceutically acceptable amount of a compound of claim 1.

22. A method of treating or preventing disorders selected from the group consisting of a CNS disorder, depression, a sleep disorder, an autonomic dysfunction a cardiovascular disorder, a renal disorder, incontinence, and cancer, tumor growth, and diabetes comprising:
identifying a subject in need of said treating or preventing; and
administering to the subject a pharmaceutically effective amount of a compound of claim 1.

23. The method of claim 22, wherein the CNS disorder is selected from group consisting of Parkinson's Disease, Alzheimer's Disease, amylotrophic lateral sclerosis, muscular dystrophy, childhood spinal muscular atrophy, progressive spinal muscular atrophy and progressive bulbar palsy, OPCA, ADHD, and schizophrenia.

24. The method of claim 22, wherein the cardiovascular disorder is selected from the group consisting of heart failure, atherosclerosis, hypertension and hypotensive states related to shock, sepsis, major surgery, congestive heart, and pulmonary disorders.

25. The method of claim 22, wherein the sleep disorder is selected from the group consisting of insomnia and narcolepsy.

26. The method of claim 22, wherein the autonomic dysfunction is Shy Drager syndrome.

27. The method of claim 22, wherein the compound is selected from the group consisting of:
[3-(4-Chlorophenyl)-3-(4-methylbenzyloxypropyl]-N,N-dimethyl amine (3a); and
[3-(4-Chlorophenyl)-3-(2-methoxybenzyloxypropyl]-N,N-dimethyl amine (3b);
as a single isomer, a mixture of isomers, or a as a racemic mixture of isomers;
as a solvate or polymorph; or as metabolite or a pharmaceutically acceptable salt or prodrug thereof.

28. The method of claim 22, wherein the compound is selected from the group consisting of:
1-(4-Chlorophenyl)-3-dimethylamino-propyl 2-methyl-benzoate HCl (4a)
1-(4-Chlorophenyl)-3-dimethylamino-propyl 2-ethyl-benzoate HCl (4b);
1-(4-Chlorophenyl)-3-dimethylamino-propyl 4-methyl-benzoate HCl (4c);
1-(4-Chlorophenyl)-3-dimethylamino-propyl 2,3-dimethyl-benzoate HCl (4d);
1-(4-Chlorophenyl)-3-dimethylamino-propyl 3-methoxy-2-methyl-benzoate HCl (4e);
1-(4-Chlorophenyl)-3-dimethylamino-propyl 3-chloro-2-methyl-benzoate HCl (4f);
1-(4-Chlorophenyl)-3-dimethylamino-propyl 4-bromo-2-methyl-benzoate HCl (4g);
1-(4-Chlorophenyl)-3-dimethylamino-propyl 2,5-dimethyl-benzoate HCl (4h);
1-(4-Chlorophenyl)-3-dimethylamino-propyl 2,4,5-trimethyl-benzoate HCl (4i);
1-(4-Chlorophenyl)-3-dimethylamino-propyl 3-methyl-thiophene-2-carboxylate HCl (4j);
1-(4-Chlorophenyl)-3-dimethylamino-propyl naphthalene-1-carboxylate HCl (4k);
1-(4-Chlorophenyl)-3-dimethylamino-propyl naphthalene-2-carboxylate HCl (4l);
1-(4-Chlorophenyl)-3-dimethylamino-propyl 4-phenyl-benzoate HCl (4m);
1-(4-Chlorophenyl)-3-dimethylamino-propyl 1-methyl-indole-2-carboxylate HCl (4n);
1-(4-Chlorophenyl)-3-dimethylamino-propyl 1-methyl-indole-3-carboxylate HCl (4o);
1-(4-Chlorophenyl)-3-dimethylamino-propyl 4-methoxy-benzoate oxalate (4p); and
1-(4-Chlorophenyl)-3-dimethylamino-propyl 4-trifluoromethyl-benzoate oxalate (4q);
as a single isomer, a mixture; of isomers, or a as a racemic mixture of isomers; as a solvate or polymorph; or as metabolite or a pharmaceutically acceptable salt or prodrug thereof.

29. The method of claim 22, wherein the compound is selected from the group consisting of:
N-[1-(4-Chlorophenyl)-3-dimethylamino-propyl]-benzamide HCl (5a);
N-[1-(4-Chlorophenyl)-3-dimethylamino-propyl]-2-methyl-benzamide oxalate (5b);
N-[1-(4-Chlorophenyl)-3-dimethylamino-propyl]-2-ethyl-benzamide oxalate (5c);
N-[1-(4-Chlorophenyl)-3-dimethylaminopropyl]-4-methoxy-benzamide oxalate (5d);
N-[1-(4-Chlorophenyl-3-dimethylaminopropyl]-4-dimethylamino-benzamide oxalate (5e);
N-[1-(4-Chlorophenyl)-3-dimethylamino-propyl]-2,3-dimethyl-benzamide oxalate (5f);
N-[1-(4-Chlorophenyl)-3-dimethylamino-propyl]-3-methoxy-2-methyl-benzamide oxalate (5g);
N-[1-(4-Chlorophenyl)-3-dimethylamino-propyl]-3-chloro-2-methyl-benzamide oxalate (5h);
N-[1-(4-Chlorophenyl)-3-dimethylamino-propyl]-2,4-dimethyl-benzamide oxalate (5i);
N-[1-(4-Chlorophenyl)-3-dimethylamino-propyl]-2,5-dimethyl-benzamide oxalate (5j);
N-[1-(4-Chlorophenyl)-3-dimethylamino-propyl]-6-chloro-2-methyl-benzamide oxalate (5k);
N-(1-(4-chlorophenyl)-3-(dimethylamino)propyl)benzo[d][1,3]dioxole-5-carboxamide oxalate (5l);
N-[1-(4-Chlorophenyl)-3-dimethylaminopropyl]-2,4,5-trimethyl-benzamide oxalate (5m);
N-[1-(4-Chlorophenyl)-3-dimethylamino-propyl]-2-naphthyl-carboxamide oxalate (5n);
N-[1-(4-Chlorophenyl)-3-dimethylamino-propyl]-4-phenyl-benzamide oxalate (5o);
(−)-N-[1-(4-Chlorophenyl)-3-dimethylamino-propyl]-4-phenyl-benzamide oxalate ((−)-5o);
(+)-N-[1-(4-Chlorophenyl)-3-dimethylamino-propyl]-4-phenyl-benzamide oxalate ((+)-5o);
N-[1-(4-Chlorophenyl)-3-dimethylaminopropyl]-4-phenoxy-benzamide oxalate (5p);
N-[1-(4-Chlorophenyl)-3-dimethylaminopropyl]-4-trifluoromethyl-benzamide oxalate (5q);
N-[1-(4-Chlorophenyl)-3-dimethylaminopropyl]-2-phenyl-acetamide oxalate (5r);
N-[1-(4-Chlorophenyl)-3-dimethylaminopropyl]-(R)-2-methoxy-2-phenyl acetamide oxalate (5s);
(+)-N-[1-(4-Chlorophenyl)-3-dimethylaminopropyl]-(R)-2-methoxy-2-phenyl-acetamide oxalate ((+)-5s); and
(−)-N-[1-(4-Chlorophenyl)-3-dimethylaminopropyl]-(R)-2-methoxy-2-phenyl-acetamide oxalate ((−)-5s);
as a single isomer, a mixture of isomers, or a as a racemic mixture of isomers; as a solvate or polymorph; or as metabolite or a pharmaceutically acceptable salt or prodrug thereof.

30. The method of claim 22, wherein the compound is selected from the group consisting of:
N-[1-(4-Chlorophenyl)-3-dimethylaminopropyl]-4-methyl-benzenesulfonamide oxalate (6a);
N-[1-(4-Chlorophenyl)-3-dimethylaminopropyl]-4-phenyl-benzenesulfonamide oxalate (6b); and
N-[1-(4-Chlorophenyl)-3-dimethylaminopropyl]-2-naphthyl-benzenesulfonamide oxalate (6c);
as a single isomer, a mixture of isomers, or a as a racemic mixture of isomers; as a solvate or polymorph; or as metabolite or a pharmaceutically acceptable salt or prodrug thereof.

31. The method of claim 22, wherein the compound is selected from the group consisting of:
N-[1-(4-Chlorophenyl)-3-dimethylaminopropyloxycarbonyl]-2-methylphenyl-amine (7a);
N-[1-(4-Chlorophenyl)-3-dimethylaminopropyloxycarbonyl]-3-methoxyphenyl-amine (7b);
N-[1-(4-Chlorophenyl)-3-dimethylaminopropyloxycarbonyl]-4-tert-butylphenyl-amine (7c);
N-[1-(4-Chlorophenyl)-3-dimethylaminopropyloxycarbonyl]-4-phenoxyphenyl-amine (7d); and
N-[1-(4-Chlorophenyl)-3-dimethylaminopropyloxycarbonyl]benzyl-amine (7e);
as a single isomer, a mixture of isomers, or a as a racemic mixture of isomers; as a solvate or polymorph; or as metabolite or a pharmaceutically acceptable salt or prodrug thereof.

32. The method of claim 22, wherein the compound is selected from the group consisting of:
N-[1-(4-Chlorophenyl)-3-dimethylaminopropyloxycarbonyl]-4-phenyl-amine (7f);
N-[1-(4-Chlorophenyl)-3-dimethylaminopropyloxycarbonyl]-2-naphthyl-amine (7g);
N-[1-(4-Chlorophenyl)-3-dimethylaminopropyloxycarbonyl]-4-methoxyphenyl-amine (7h); and
N-[1-(4-Chlorophenyl)-3-dimethylaminopropyloxycarbonyl]-4-trifluoromethylphenyl-amine (7i);
as a single isomer, a mixture of isomers, or a as a racemic mixture of isomers; as a solvate or polymorph; or as metabolite or a pharmaceutically acceptable salt or prodrug thereof.

33. The method of claim 22, wherein the compound is selected from the group consisting of:
1-[1-(4-Chlorophenyl)-3-dimethylaminopropyl]-3-(2-methylphenyl)-carbamide oxalate (8a);
1-[1-(4-Chlorophenyl)-3-dimethylaminopropyl]-3-(4-tert-butylphenyl)-carbamide oxalate (8c);
1-[1-(4-Chlorophenyl)-3-dimethylaminopropyl]-3-(4-phenoxyphenyl)-carbamide oxalate (8d); and
1-[1-(4-Chlorophenyl)-3-dimethylaminopropyl]-3-benzyl-carbamide oxalate (8e);
as a single isomer, a mixture of isomers, or a as a racemic mixture of isomers; as a solvate or polymorph; or as metabolite or a pharmaceutically acceptable salt or prodrug thereof.

34. The method of claim 22, wherein the compound is selected from the group consisting of:
1-[1-(4-Chlorophenyl)-3-dimethylaminopropyl]-3-(4-phenylphenyl)-carbamide oxalate (8f);
1-[1-(4-Chlorophenyl)-3-dimethylaminopropyl]-3-(2-naphthyl)-carbamide oxalate (8g);
1-[1-(4-Chlorophenyl)-3-dimethylaminopropyl]-3-(4-methoxyphenyl)-carbamide oxalate (8h); and
1-[1-(4-Chlorophenyl)-3-dimethylaminopropyl]-3-(4-trifluoromethylphenyl)-carbamide oxalate (8i);
as a single isomer, a mixture; of isomers, or a as a racemic mixture of isomers; as a solvate or polymorph; or as metabolite or a pharmaceutically acceptable salt or prodrug thereof.

35. The method of claim 22, wherein the compound is selected from the group consisting of:
N-[1-(4-Chlorophenyl)-3-dimethylamino-propyl]-2-phenylacetamide HCl (A1);
N-[1-(4-Chlorophenyl)-3-dimethylamino-propyl]-2-(4-trifluoromethylphenyl)acetamide HCl (A2);
N-[1-(4-Chlorophenyl)-3-dimethylamino-propyl]-2-(4-methoxyphenyl)acetamide HCl (A3);
N-[1-(4-Chlorophenyl-3-dimethylamino-propyl]-3-phenyl-propionamide HCl (A4);
N-[1-(4-Chlorophenyl)-3-dimethylamino-propyl]-3-(4-trifluoromethylphenyl)-propionamide HCl (A5);
N-[1-(4-Chlorophenyl)-3-dimethylamino-propyl]-3-(4-methoxyphenyl)propanamide HCl (A6);
N-[1-(4-Chlorophenyl)-3-dimethylamino-propyl]-3-cinnamic amide HCl (A7);
N-[1-(4-Chlorophenyl)-3-dimethylamino-propyl]-4-trifluoromethyl-cinnamic amide HCl (A8);
N-[1-(4-Chlorophenyl)-3-dimethylamino-propyl]-4-methoxy-cinnamic amide HCl (A9); and
N-[1-(4-Chlorophenyl)-3-dimethylamino-propyl]-3-phenylpropiolic amide HCl (A10);
as a single isomer, a mixture of isomers, or a as a racemic mixture of isomers; as a solvate or polymorph; or as metabolite or a pharmaceutically acceptable salt or prodrug thereof.

36. The method of claim 22, wherein the compound is selected from the group consisting of:
N-[3-Dimethylamino-1-(4-methylphenyl)propyl]-2-phenylacetamide HCl (B1);
N-[3-Dimethylamino-1-(4-methylphenyl)propyl]-2-(4-trifluoromethylphenyl)acetamide HCl (B2);
N-[3-Dimethylamino-1-(4-methylphenyl)propyl]-2-(4-methoxyphenyl)acetamide HCl (B3);
N-[3-Dimethylamino-1-(4-methylphenyl)propyl]-3-phenylpropionamide HCl (B4);
N-[3-Dimethylamino-1-(4-methylphenyl)propyl]-3-(4-trifluoromethylphenyl)propionamide HCl (B5);
N-[3-Dimethylamino-1-(4-methylphenyl)propyl]-3-(4-methoxyphenyl)propionamide HCl (B6);
N-[3-Dimethylamino-1-(4-methylphenyl)propyl]cinnamic amide (B7);
N-[3-Dimethylamino-1-(4-methylphenyl)propyl]-4-trifluoromethyl-cinnamic amide HCl (B8);
N-[3-Dimethylamino-1-(4-methylphenyl)propyl]-4-methoxy-cinnamic amide HCl (B9); and
N-[3-Dimethylamino-1-(4-methylphenyl)propyl]-3-phenylpropiolic amide (B10);
as a single isomer, a mixture of isomers, or a as a racemic mixture of isomers; as a solvate or polymorph; or as metabolite or a pharmaceutically acceptable salt or prodrug thereof.

37. The method of claim 22, wherein the compound is selected from the group consisting of:
N-[3-Dimethylamino-1-(2-naphthyl)propyl]-2-phenylacetamide HCl (C1);
N-[3-Dimethylamino-1-(2-naphthyl)propyl]-2-(4-trifluoromethylphenyl)acetamide HCl (C2);
N-[3-Dimethylamino-1-(2-naphthyl)propyl]-2-(4-methoxyphenyl)acetamide HCl (C3);
N-[3-Dimethylamino-1-(2-naphthyl)propyl]-3-phenyl-propionamide HCl (C4);
N-[3-Dimethylamino-1-(2-naphthyl)propyl]-2-(4-trifluoromethylphenyl)propionamide HCl (C5);
N-[3-Dimethylamino-1-(2-naphthyl)propyl]-3-(4-methoxyphenyl-propion amide HCl (C6);
N-[3-Dimethylamino-1-(2-naphthyl)propyl]-3-cinnamic amide HCl (C7);
N-[3-Dimethylamino-1-(2-naphthyl)propyl]-4-trifluoromethyl-cinnamic amide HCl (C8);
N-[3-Dimethylamino-1-(2-naphthyl)propyl]-4-methoxy-cinnamic amide HCl (C9); and
N-[3-Dimethylamino-1-(2-naphthyl)propyl]-3-phenylpropiolic amide HCl (C10);
as a single isomer, a mixture of isomers, or a as a racemic mixture of isomers; as a solvate or polymorph; or as metabolite or a pharmaceutically acceptable salt or prodrug thereof.

38. A method of identifying a compound which is an agonist, inverse agonist, or antagonist of the UII receptor, the method comprising:
contacting a UII receptor with at least one test compound of Formula I; and
determining any increase or decrease in activity level of said UII receptor.
